(19) United States
(12) Patent Application Publication
(10) Pub. No.: US 2022/0409722 A1
(43) Pub. Date: Dec. 29, 2022

(54) FENTANYL HAPTEN, FENTANYL HAPTEN-CONJUGATES, AND METHODS FOR MAKING AND USING

(71) Applicants: REGENTS OF THE UNIVERSITY OF MINNESOTA, Minneapolis, MN (US); HENNEPIN HEALTHCARE RESEARCH INSTITUTE, Minneapolis, MN (US); RTI INTERNATIONAL, Research Park, NC (US)

(72) Inventors: Marco Pravetoni, Minneapolis, MN (US); Scott Runyon, Research Park, NC (US)

(21) Appl. No.: 17/774,906

(22) PCT Filed: Nov. 6, 2020
(86) PCT No.: PCT/US2020/059490

§ 371 Date: May 6, 2022

Related U.S. Application Data
(60) Provisional application No. 62/932,757, filed on Nov. 8, 2019.

(51) Int. Cl. A61K 39/385; A61K 39/00; A61P 39/00

(52) U.S. Cl. CPC A61K 39/385; A61K 39/0013; A61P 39/00; A61K 2039/6081

(57) ABSTRACT

This disclosure describes a fentanyl hapten, a fentanyl hapten-carrier conjugate, methods of making the fentanyl hapten and the fentanyl hapten-carrier conjugate, and methods of using the fentanyl hapten and the fentanyl hapten-carrier conjugate. The fentanyl hapten-carrier conjugate may be used, for example, as a prophylactic vaccine to counteract toxicity from exposure to fentanyl and its analogues. In some embodiments, the fentanyl hapten-carrier conjugate or a composition including the fentanyl hapten-carrier conjugate may be used in an anti-opioid vaccine.

### CROSS-REFERENCE TO RELATED APPLICATIONS

This application claims the benefit of U.S. Provisional Application Ser. No. 62/932,757, filed Nov. 8, 2019, which is incorporated by reference herein.

### GOVERNMENT FUNDING

This invention was made with government support under DA048386 and DA038876 awarded by the National Institutes of Health. The government has certain rights in the invention.

### BACKGROUND

Opioid use disorders (OUDs) and the fatal overdose epidemic are a growing public health burden. The incidence of fatal overdoses from synthetic opioids increased 100% from 2015 to 2016, largely driven by illicitly manufactured fentanyl. Fentanyl, an extremely potent synthetic opioid, and its analogs have been involved in more than 50% of opioid-related fatalities in the United States. Fentanyl has been increasingly used to adulterate heroin, cocaine, and counterfeit prescription pills, leading to an increase in opioid-induced fatal overdoses in the United States, Canada, and Europe.

Fentanyl analogs have also been used by Russian Special Forces in the Moscow theater hostage situation, which resulted in at least 150 fatal overdoses in both civilian and terrorists. It is feared that fentanyl and its analogs may be used in mass casualty incidents, deliberate poisoning, and chemical attacks against civilians, military, and at-risk professionals.

### SUMMARY OF THE INVENTION

This disclosure describes a fentanyl hapten, a fentanyl hapten-carrier conjugate, methods of making the fentanyl hapten and fentanyl hapten-carrier conjugate, and methods of using the fentanyl hapten and fentanyl hapten-carrier conjugate including, for example, in a prophylactic or therapeutic vaccine to counteract toxicity from exposure to fentanyl and its analogues.

In one aspect, this disclosure describes a fentanyl hapten-carrier conjugate including a fentanyl hapten including

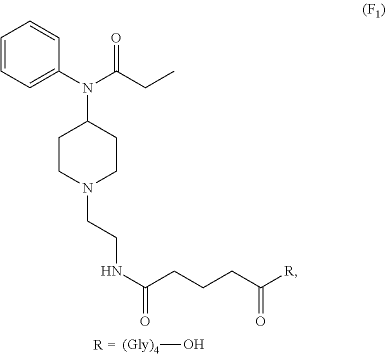

and an immunogenic carrier, wherein the fentanyl hapten is conjugated to the immunogenic carrier.

In another aspect, this disclosure describes a composition that includes the fentanyl hapten-carrier conjugate.

In a further aspect, this disclosure describes a method of making the fentanyl hapten-carrier conjugate or the composition that includes the fentanyl hapten-carrier conjugate.

In yet another aspect, this disclosure describes a method that includes administering the fentanyl hapten-carrier conjugate or the composition that includes the fentanyl hapten-carrier conjugate to a subject.

In an additional aspect, this disclosure describes a fentanyl hapten including

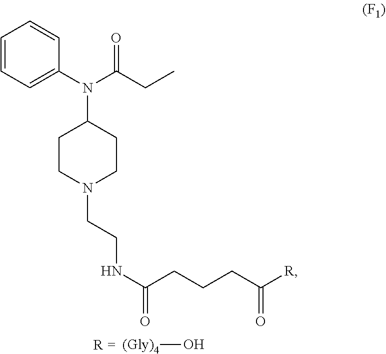

wherein the F₁ has a differential scanning calorimetry (DSC) thermogram exhibiting an endothermic event having a melt maxima temperature in a range of 110 degrees Celsius (° C.) to 130° C.; wherein the F₁ has a DSC thermogram exhibiting an endothermic event having a melt maxima temperature in a range of 175° C. to 185° C.; wherein the F1 has a decompensation temperature of at least 250° C., as measured by thermogravimetric analysis (TGA); or wherein the F1 has a haptenation ratio to BSA, of at least 10, at least 15, at least 20; or more than 20; or a combination thereof.

In another aspect, this disclosure describes a method of making a fentanyl hapten including

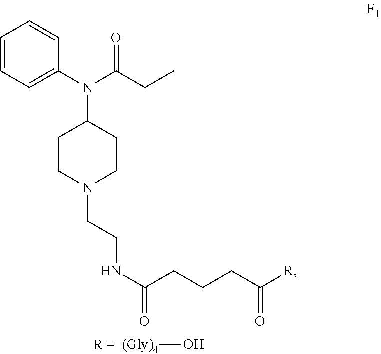

wherein the method includes the synthesis shown in Scheme 2 (FIG. 1D).

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (for example, 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A shows the structure of fentanyl. FIG. 1B shows the structure of a fentanyl-based hapten containing a tetraglycine linker (referred to herein as F, F₁, or F(Gly)₄) which has a chemical formula of C₂₉H₄₂LiN₇O₈ and a molecular weight of 623.64. FIG. 1C shows Scheme 1, a synthetic pathway to obtain the F₁ hapten [8]. Reactants a. 2-(Boc-amino)ethylbromide, K₂CO₃, acetonitrile, 80° C., 88%; b. aniline, AcOH, NaBH₃CN, CH₂Cl₂, 80° C., 86%; c. propionyl chloride, DIPEA, CH₂Cl₂, rt, 95%; d. TFA/CH₂Cl₂ (2/8-v/v), rt, quantitative; e. glutaric anhydride, pyridine, CH₂Cl₂, rt, quantitative; f. (Gly)₄-OtBu, HBTU, DIPEA, CH₂Cl₂, rt, 74%; TFA/CH₂Cl₂ (2/8-v/v), rt, 92%; g. TFA/CH₂Cl₂ (2/8-v/v), rt, 92%. FIG. 1D shows Scheme 2, a synthetic pathway to obtain the F₁ hapten, as further described in Example 5. FIG. 1E shows the structures of F₁, fentanyl, sufentanil, and acetylfentanyl. F₁ is missing one of the rings of the structures of fentanyl, sufentanil, and acetylfentanyl.

FIG. 2A-FIG. 2B show the results of Experiment 1 of Example 1: Active immunization with F₁ hapten conjugated to native keyhole limpet hemocyanin (F₁-KLH), reduces fentanyl-induced antinociceptive effects and increases serum fentanyl concentrations in mice relative to immunization with native keyhole limpet hemocyanin (KLH) alone. FIG. 2A shows vaccination with F₁-KLH significantly reduced fentanyl-induced hotplate antinociception by 60% (Mean±Standard Error of Mean (SEM)). FIG. 2B shows vaccination with F₁-KLH increased serum fentanyl concentrations (Mean±Standard Deviation (SD)) compared to controls 30 minutes after a 0.05 mg/kg subcutaneous (s.c.) dose of fentanyl. Numbers above bars represent the percent difference from controls. **p<0.01, ***p<0.001 compared to controls using unpaired t tests with Welsh's correction. n=6/group.

FIG. 3A-FIG. 3E show the results of Experiment 2 of Example 1: selectivity and pharmacokinetic efficacy of F-KLH in rats. FIG. 3A shows vaccination with F₁-KLH significantly reduced fentanyl-induced hotplate antinociception by 93% at 30 minutes after a 0.035 mg/kg s.c. dose of fentanyl. FIG. 3B and FIG. 3C shows F₁-KLH had no effect on heroin- or oxycodone-induced antinociception 30 minutes after a 1 mg/kg or 2.25 mg/kg dose of heroin or oxycodone, respectively. FIG. 3D shows that serum fentanyl concentrations were significantly increased compared to controls 4 minutes after a 1-minute 0.05 mg/kg i.v. infusion of fentanyl. FIG. 3E shows brain fentanyl concentrations were significantly decreased compared to controls 4 minutes after a 1-minute 0.05 mg/kg i.v. infusion of fentanyl. Numbers above bars represent the percent difference from controls. *p<0.05, ***p<0.001 compared to controls. Mean±SD (FIG. 3D and FIG. 3E), Mean±SEM (FIG. 3A, FIG. 3B, and FIG. 3C), n=12/group (FIG. 3A, FIG. 3D, and FIG. 3E) and n=6/group (FIG. 3B and FIG. 3C).

FIG. 4A-FIG. 4F show fentanyl dose-response and the effects of F₁ hapten conjugated to a GMP-grade subunit KLH (F₁-sKLH) on hotplate antinociception, respiratory depression, and bradycardia in rats. Fentanyl was administered s.c. every 15 minutes at increasing doses in non-immunized rats and the doses listed are the cumulative dose received. FIG. 4A shows the effect of fentanyl on hotplate antinociception. Latency to respond is capped at 60 seconds. Naloxone (0.1 mg/kg, s.c.) was administered 15 minutes after the final fentanyl dose. FIG. 4B shows the effect of fentanyl on respiratory depression, measured as arterial oxygenation (SaO2). FIG. 4C shows the effect of fentanyl on heart rate. **p<0.01, ***p<0.001 for the difference between values compared to baseline. FIG. 4D shows vaccine effects on fentanyl-induced antinociception. FIG. 4E shows vaccine effects on fentanyl-induced respiratory depression, measured as arterial oxygenation (SaO2). FIG. 4F shows vaccine effects on fentanyl-induced decreases in heart rate. *p<0.05, **p<0.01, ***p<0.001 for differences from baseline within groups. #p<0.05, ##p<0.01, and ###p<0.001 for the difference between groups at each dose. There were no differences between groups in latency to respond, SaO2, or heart rate following naloxone treatment. Mean±SD; n=8/group.

FIG. 5A-FIG. 5B show the results of Experiment 4 of Example 1: F₁-sKLH alters fentanyl distribution in serum and to the brain. FIG. 5A shows F₁-sKLH vaccination increased serum fentanyl distribution 30 minutes after receiving a cumulative 0.1 mg/kg s.c. fentanyl dose. FIG. 5B shows F₁-sKLH vaccination decreased brain fentanyl distribution by 73% 30 minutes after receiving a cumulative 0.1 mg/kg s.c. fentanyl dose. Numbers above bars represent the percent difference from controls. Mean±SD, p<0.001 compared to controls using unpaired t tests with Welch's correction.

FIG. 6A-FIG. 6B show characterization of F₁ hapten activity at the Mu Opioid Receptor (MOR). FIG. 6A shows the F₁ hapten (referred to in this figure as “hapten 1”) does not contain the N-phenylethyl moiety that is critical for activity at the MOR; this moiety in F₁ is replaced with a tetraglycine peptidic linker that yields a hapten that has no functional activity at the MOR when tested in a calcium mobilization assay involving Chinese Hamster Ovary (CHO) cells co-expressing the human MOR and G_{α16}, a promiscuous G protein. FIG. 6B shows the F₁ hapten has no functional agonist activity at the MOR, most likely due to the extended peptidic linker and lack of an N-phenylethyl substituent.

FIG. 7A-FIG. 7C show characterization of F₁ hapten conjugated to BSA and sKLH carrier proteins. FIG. 7A-FIG. 7B show exemplary MALDI-TOF traces of unconjugated BSA (FIG. 7A) and F₁ hapten conjugated to BSA (F₁-BSA) with haptenation (also referred to as haptenization) ratio (HR) of 23 (FIG. 7B). FIG. 7A-FIG. 7B report molecular weight (MW). FIG. 7C shows exemplary Dynamic Light Scattering (DLS) traces of unconjugated carrier protein (sKLH) and F₁ hapten conjugated to sKLH. FIG. 7C reports size (nm) and Polydispersity Index (PDI).

FIG. 8A-FIG. 8C show in vivo efficacy of conjugated F₁ hapten in BALB/c mice. Haptens were conjugated to either sKLH or EcoCRM (Fina Biosolutions, Rockville, Md., designated as CRM₁) by carbodiimide (EDAC) chemistry. Conjugates were adsorbed on alum adjuvant and injected i.m. in mice on days 0, 14 and 28. Immunization induced fentanyl-specific serum IgG antibodies at day 14 (FIG. 8A) and day 34 (FIG. 8B). On day 35, mice were challenged with 0.1 mg/kg s.c. fentanyl. FIG. 8C shows immunization with both F₁-sKLH and F₁-CRM₁ effectively reduced fentanyl-induced antinociception in the hot plate test at 30-minutes post-drug challenge. Statistical symbols: ****p≤0.0001 compared to control immunized with unconjugated carrier proteins. Brackets indicate group differences.

FIG. 9A-FIG. 9D show in vivo efficacy of conjugated F₁ hapten against fentanyl in Sprague Dawley rats. F₁ hapten was conjugated to sKLH, CRM₁, or CRM₁₉₇ (PFEnex, San Diego Calif., designated as CRM₂). Conjugates were injected i.m. on days 0, 21, 42, and 63. Starting the week after the third vaccination, rats were challenged weekly with either fentanyl or sufentanil (week 1 challenge—0.075 mg/kg s.c. fentanyl; week 2 challenge—0.008 mg/kg s.c. sufentanil; week 3 challenge—0.1 mg/kg s.c. fentanyl). FIG. 9A shows all conjugates generated detectable fentanyl-specific IgG titers as measured after the 3^{rd} vaccination on day 49. In the week 1 challenge, conjugates were effective at reducing the effects of 0.075 mg/kg s.c. fentanyl: FIG. 9B shows antinociception in the hot plate test; FIG. 9C shows respiratory depression reported as percentage (%) of oxygen saturation measured by oximetry; and FIG. 9D shows bradycardia reported as heart rate (beats per minute, bpm) measured by oximetry. Two subsequent challenges (week 2 challenge and week 3 challenge) are shown in FIG. 10 and FIG. 11. During the week 1 challenge, the F₁-CRM₁ or F₁-CRM₂ were more effective than F₁-sKLH. Statistical symbols: *p≤0.05, **p≤0.01, ***p≤0.001, and ****p≤0.0001 compared to control. Brackets indicate group differences. #p≤0.05 compared to F₁-sKLH.

FIG. 10A-FIG. 10D show in vivo efficacy of conjugated F₁ hapten against sufentanil in rats. Two weeks after the third vaccination (as described in the figure legend of FIG. 9), rats were challenged with 0.008 mg/kg s.c. sufentanil. The F₁-CRM₁ or F₁-CRM₂ conjugates were more effective than either control or F₁-sKLH in reducing sufentanil-induced antinociception in the hot plate test measured at: 15 minutes (FIG. 10A), 30 minutes (FIG. 10B), 45 minutes (FIG. 10C), and 60 minutes (FIG. 10D) post-drug challenge. A subsequent and final challenge is shown in FIG. 11. Statistical symbols: *p≤0.05 compared to control, and #p≤0.05 compared to F₁-sKLH.

FIG. 11A-FIG. 11C show in vivo efficacy of conjugated F₁ hapten against a higher dose of fentanyl in rats. Three weeks after the third vaccination (as described in the figure legend of FIG. 9), rats immunized with conjugates containing the F₁ hapten were challenged with 0.1 mg/kg s.c. fentanyl. The F₁-CRM₁ or F₁-CRM₂ conjugates were more effective than either control or F₁-sKLH in reducing fentanyl-induced respiratory depression (FIG. 11A) and bradycardia (FIG. 11B) as measured by oximetry. FIG. 11C shows all conjugates were effective in reducing the distribution of fentanyl to the brain at 60 minutes after the initial drug challenge. Statistical symbols: *p≤0.05, **p≤0.01, and ****p≤0.0001 compared to control, and #p≤0.05 compared to F₁-sKLH.

FIG. 12A-FIG. 12B show exemplary results of active immunization on reduced fentanyl intravenous self-administration (FSA) in rats. First, rats were trained to self-administer fentanyl (1 μg/kg/infusion) under a fixed-ratio (FR) 3 schedule during daily 120-minute sessions. Once fentanyl self-administration (FSA) stabilized, rats were immunized i.m. with either CRM₁ or F₁-CRM₁ (n=6/group) on days 0, 21, 42, and 63. FIG. 12A shows FSA declined by more than 50% in the F₁-CRM₁ group after the 4^{th} immunization compared to its baseline level prior to vaccination (47.7±11.7 mean±SEM), whereas the control group showed no change. FIG. 12B shows when rats were challenged with a dose-reduction protocol, rats vaccinated with F₁-CRM₁ decreased their intake over time, while control CRM₁ increased their mean infusion/session to compensate for the dose reduction. The fentanyl dose was reduced every Monday for four consecutive weeks, and FIG. 12B shows the mean of the last two sessions of the week (Thursday and Friday) at each descending dose. Statistical symbols: **p≤0.01 compared to control, and #p≤0.05 compared to pre-immunization baseline.

FIG. 13A-FIG. 13B show that in vivo efficacy of an anti-fentanyl vaccine is not affected by environmental conditions. F₁-sKLH was tested in mice housed in either specified-pathogen free (SPF) or conventional housing conditions. Conjugates were adsorbed on alum adjuvant and injected in mice i.m. on days 0, 14, and 28. A week after the 3^{rd} vaccination, mice were challenged with 0.05 mg/kg s.c. fentanyl. Housing conditions had no impact on efficacy of F₁-sKLH. The F₁ conjugate was effective in reducing fentanyl-induced analgesia in the hot-plate test at 30-minutes post-drug challenge (FIG. 13A), and fentanyl distribution to the brain (FIG. 13B). Statistical symbols: *p≤0.05, ***p≤0.001, ****p≤0.0001 compared to its respective housing group sKLH control.

FIG. 14A-FIG. 14D show that the efficacy of an anti-fentanyl vaccine is enhanced by blockade of interleukin 4 (IL-4). Male BALB/c mice were immunized with either unconjugated carrier protein (mixture of sKLH and CRM₁), F₁-sKLH, or F₁-CRM₁ on days 0, 14, and 28. Before and after the 1^{st} immunization, mice were given either saline or an anti-IL-4 neutralizing monoclonal antibody (αIL-4). FIG. 14A shows serum IgG₁ antibody titers; FIG. 14 B shows serum IgG₂ₐ antibody titers; FIG. 14C shows serum fentanyl concentration; and FIG. 14D shows brain fentanyl after challenge with 0.05 mg/kg s.c. fentanyl. Statistical symbols: **p≤0.01, ****p≤0.0001 compared to control, or as indicated by brackets.

FIG. 15A shows an exemplary profile resulting from thermogravimetric analysis (TGA) of F₁, as further described in Example 3. FIG. 15B shows the exemplary thermograms from differential scanning calorimetry (DSC) analysis of F₁, as further described in Example 3. FIG. 15C-FIG. 15F show representative MALDI-TOF traces of conjugates and unconjugated carrier proteins. FIG. 15C. BSA and F₁-BSA with an haptenation ratio (HR) of 23. Representative Dynamic Light Scattering (DLS) traces of F₁-sKLH (FIG. 15D), F₁-CRM₁ (FIG. 15E), and F₁-CRM₂ (FIG. 15F). FIG. 15C-FIG. 15F show conjugates at T=0 and T=1 month after storage at +4° C. MI is mean diameter of the intensity distribution, PDI is the polydispersity index.

FIG. 16 shows pre-existing immunity to the carrier does not interfere, or minimally interferes with vaccine-induced antibody responses against fentanyl in mice. Male BALB/c mice were first immunized with sKLH, CRM₁, CRM₂ or saline as control on day −14 (2 weeks before the first immunization). Then mice were immunized with F₁-sKLH, F₁-CRM₁, and F₁-CRM₂ on days 0, 14 and 28, and fentanyl-specific antibodies analyzed at day 35. Previous exposure to sKLH and CRM₂ did not affect antibody responses to vaccination. Although minimal, significant interference with F₁-CRM₁ was detected. Brackets indicated analysis by unpaired t-test to assess effect of pre-immunization with each individual carrier. Symbol: *p<0.05.

FIG. 17A-FIG. 17K show immunization against fentanyl does not interfere with anesthesia protocols. Sprague Dawley rats (n=6, each group) were immunized i.m. with either CRM₁ or F₁-CRM₁ on days 0, 21, 42 and 63. From day 49, rats were challenged weekly with 2% inhaled isoflurane, 0.25 mg/kg dexmedetomidine (reversed by 1 mg/kg atipamezole), 75 mg/kg ketamine, 100 mg/kg propofol, or 0.05 mg/kg fentanyl as control. Induction of anesthetic efficacy was initially monitored by the loss of righting reflex, and then measured by respiratory depression reported as percent (%) oxygen saturation and bradycardia reported as heart rate (bpm), both measured by pulse oximetry. Results were equivalent in CRM₁ or F₁-CRM₁ groups, confirming the selectivity of these vaccines. FIG. 17A-FIG. 17C show exemplary results with dexmedetomidine, FIG. 17D-FIG. 17E show exemplary results with ketamine, FIG. 17F shows exemplary results with propofol, FIG. 17G-FIG. 17H show exemplary results with isoflurane, and FIG. 17I-FIG. 17K show exemplary results with fentanyl as positive control. Statistical symbols: *p≤0.05, **p≤0.01.

FIG. 18A-FIG. 18F show immunization against fentanyl does not interfere with off-target opioids used in pain management, treatment of opioid use disorder, or reversal of opioid overdose (with, for example, naloxone). Sprague Dawley rats (n=6, each group) were immunized i.m. with either CRM₁ or F₁-CRM₁ on days 0, 21, and 42. One week after the third vaccination rats were challenged s.c. weekly with: oxycodone (2.25 mg/kg—FIG. 18A and FIG. 18C), heroin (0.9 mg/kg—FIG. 18B and FIG. 18D), methadone (2.25 mg/kg—FIG. 18E), or fentanyl (0.1 mg/kg—FIG. 18F) as control. FIG. 18A-FIG. 18D show oxycodone and heroin effects were reversed by naloxone (0.1 mg/kg, s.c.). Drug-induced antinociception, or its reversal by naloxone, was assessed in the hotplate test of analgesia. Vaccination did not interfere with the antinociceptive effects of oxycodone, heroin or methadone. The effect of naloxone was preserved in both control and vaccinated groups. Statistical symbols: *p≤0.05, **p<0.01, ****p<0.0001.

FIG. 19A-FIG. 19D shows efficacy of a fentanyl vaccine against cumulative fentanyl dosing in rats. Sprague Dawley rats were immunized with either CRM control or the F₁-CRM₂ conjugate adsorbed on alum (n=9 per group). Conjugates were injected i.m. on days 0, 21, 42 and 63. A week after the 4^{th} vaccination, rats were challenged with a dose of 0.25 mg/kg fentanyl delivered s.c. every 15 minutes to a final cumulative dose of 2.25 mg/kg or until respiratory/cardiac arrest. The F₁-CRM₂ conjugate was effective in shifting: FIG. 19A shows the ED50 for respiratory depression as measured by oxygen saturation (%); FIG. 19B shows the ED50 for bradycardia as measure by heart rate (bpm) over the course of the experiment; and FIG. 19C shows fentanyl-induced antinociception expressed as MPE % on a hotplate. FIG. 19D shows that at the final cumulative dose of 2.25 mg/kg, rats in the control group experience statistically significant increase in cardiac and/or respiratory arrest. FIG. 19A-FIG. 19C. Data were analyzed using a two-way ANOVA (mixed model) paired with Sidak's multiple comparisons test. FIG. 19D. Data were expressed as percentage of alive or death rats and analyzed by both Chi-square and Fisher's exact tests. Statistical symbols: *,**, **** indicate p≤0.05, 0.01, and 0.0001, respectively, compared to control.

FIG. 20A-FIG. 20C show efficacy of vaccines containing the F₁ hapten against acetylfentanyl in rats. Rats were immunized with control or F₁-CRM₂ and then were challenged with varying doses of acetylfentanyl to test whether F₁ induces polyclonal antibodies that cross-react with acetylfentanyl. Rats were challenged with 0.5 mg/kg s.c. acetylfentanyl then re-challenged with 1 mg/kg s.c. acetylfentanyl and drug-induced hot plate antinociception (FIG. 20A), respiratory depression (FIG. 20B), and bradycardia (FIG. 20C) were measured.

### DETAILED DESCRIPTION OF ILLUSTRATIVE EMBODIMENTS

This disclosure describes a fentanyl hapten, a fentanyl hapten-carrier conjugate, methods of making the fentanyl hapten and the fentanyl hapten-carrier conjugate, and methods of using the fentanyl hapten and the fentanyl hapten-carrier conjugate including, for example, as a therapeutic vaccine or a prophylactic vaccine to counteract toxicity from exposure to fentanyl and its analogues.

### Fentanyl

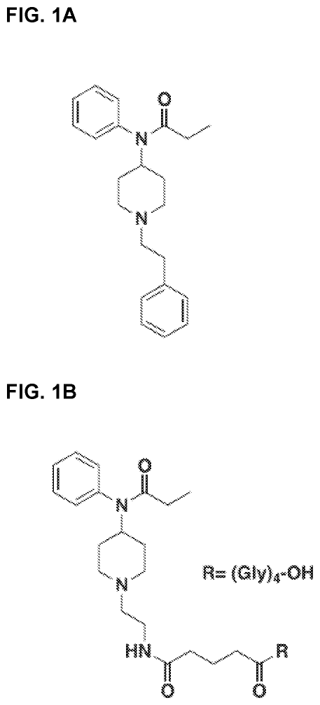

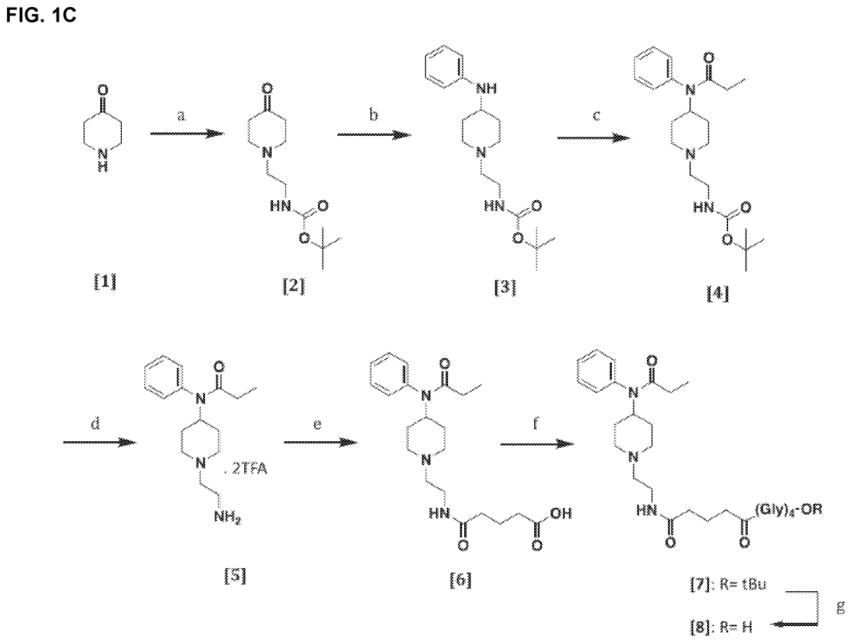

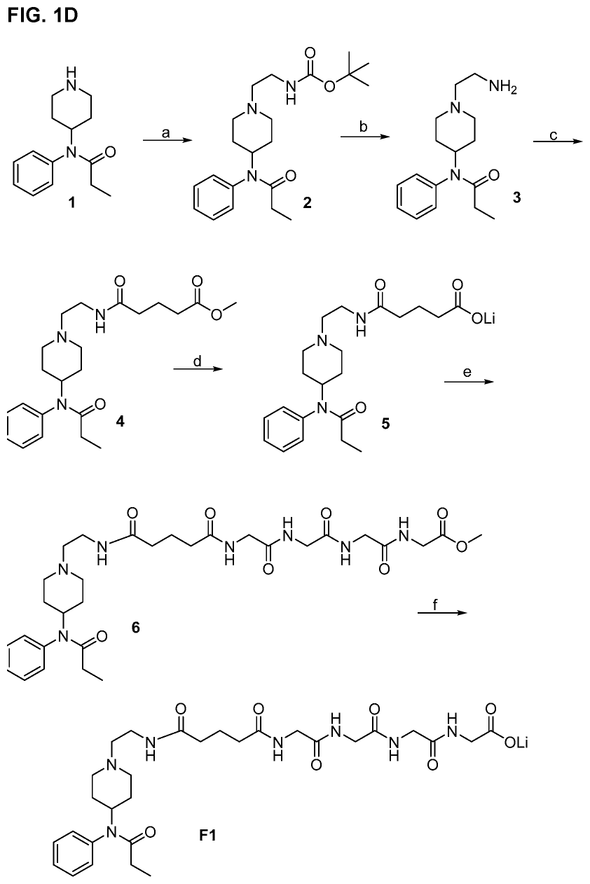

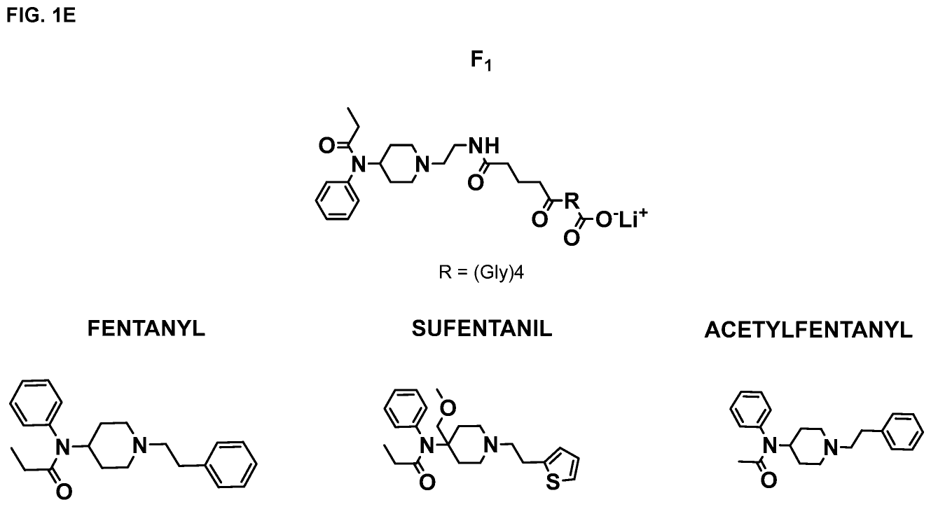

Fentanyl (FIG. 1A) is a schedule II opioid agonist with an extremely high in vivo potency 100-200 times than that of morphine. Fentanyl has been increasingly used as an adulterant in heroin and counterfeit prescription opioids because of its potency, ease of chemical feasibility, and low manufacturing costs. Overdose deaths from heroin or hydrocodone/acetaminophen laced with fentanyl or its derivatives carfentanil, acetylfentanyl, and alfentanil have been increasingly reported in North America. The presence of fentanyl was also recorded in fatal overdoses related to cocaine, benzodiazepines, antidepressants, and other counterfeit or illicit drugs. In addition to fentanyl misuse in patients with OUD, fentanyl and its derivatives have also been used as chemical agents for incapacitation in military scenarios. Finally, fentanyl and its analogs pose a potential risk for law enforcement officials, first responders, airport or custom personnel and their canine units.

The development of novel treatment modalities to reduce the incidence or severity of accidental overdoses from fentanyl and its derivatives could have substantial public health impact.

### Current Treatments for Fentanyl Overdose

The current treatment for fentanyl overdose is naloxone, an opioid antagonist. Distribution of naloxone to high-risk populations is being expanded in the US and has been shown to be a cost-effective strategy for decreasing overdose deaths in both the US and UK. However, for naloxone to be effective, administration is required shortly after exposure and with proper technique. Due to this consideration, as well as fentanyl's potency, naloxone may not always be sufficient to rapidly reverse fentanyl-induced respiratory depression. Also, administration of naloxone may precipitate opioid-withdrawal syndrome and cause severe side effects.

In contrast, prophylactic or therapeutic vaccination against fentanyl could be a cost-effective, long-lasting intervention to reduce the incidence or severity of fentanyl overdose. Moreover, if sufficiently selective, an anti-opioid vaccine could be combined with a current pharmacological treatment for OUD and/or overdose.

### Opioid Vaccines

Opioid vaccines have been explored pre-clinically as a treatment for OUD and have been effective in rodent and non-human primate models (Stowe et al., Journal of Medicinal Chemistry 2011; 54:5195-5204; Bremer et al. J Med Chem. 2012; 55:10776-10780; Pravetoni et al., Vaccine 2012; 30:4617-4624; Matyas et al., Vaccine 2013; 31:2804-2810; Raleigh et al., The Journal of Pharmacology and Experimental Therapeutics 2013; 344:397-406; Schlosburg et al., PNAS 2013; 110:9036-9041; Bremer et al., Molecular Pharmaceutics 2014; 11:1075-1080; Raleigh et al., PloS One 2014; 9:e115696; Bremer et al., 2017; Raleigh et al., PloS One 2017; 12:e0184876). Opioid vaccines elicit opioid-specific antibodies that selectively bind to the targeted opioids in the blood and reduce their distribution to the brain, reducing their behavioral and toxic effects. Vaccine efficacy is greatest when the levels of antibody produced are high and the opioid dose is low. Because fentanyl is quite potent and has a relatively low toxic dose compared to other abused opioid such as heroin or oxycodone, it is a particularly attractive candidate for this approach.

A limited number of studies showed pre-clinical proof of concept for immunotherapy against fentanyl and its analogs in mice, rabbits, and dogs (Torten et al., Nature 1975; 253:565-566; Bremer et al., Angew Chem Int Ed Engl. 2016; 55:3772-3775; Hwang et al., ACS Chem Neurosci 2018; 9:1269-1275). These studies showed that fentanyl-specific antibodies reduced hotplate antinociception and fentanyl-induced respiratory depression following small fentanyl doses. These studies involved either passive immunization with polyclonal antibodies or active vaccination using complete Freund's adjuvant intradermally (Henderson et al., The Journal of Pharmacology and Experimental Therapeutics 1975; 192:489-496; Torten et al., Nature 1975) or other adjuvants administered i.p. (Bremer et al., Angew Chem Int Ed Engl. 2016; 55:3772-3775; Hwang et al., ACS Chem Neurosci 2018; 9:1269-1275), each of which may not be feasible in humans.

Although the use of active immunization with fentanyl haptens conjugated to carriers for blocking fentanyl analgesia has been previously suggested (see U.S. Publication No. 20140093525A1), the efficacy of any such vaccine and its ability to be used in combination with naloxone were unpredictable. Moreover, as described in Example 1 and Example 2, the ability of a fentanyl hapten-carrier conjugate vaccine to reduce or prevent fentanyl-induced respiratory depression and to reduce or prevent bradycardia (cardiac toxicity) were unexpected, particularly because reduction of fentanyl-induced bradycardia is unprecedented.

As described in Example 1, a study was conducted to determine the efficacy of a fentanyl vaccine including F₁ conjugated to an immunogenic carrier protein and adsorbed on alum adjuvant and administered intramuscularly (i.m.) in mice and rats. The F₁-KLH vaccine was developed using a fentanyl-based hapten (F₁) conjugated to either the native decamer keyhole limpet hemocyanin (KLH) carrier protein or the GMP-grade subunit KLH (sKLH) by means of a tetraglycine linker using carbodiimide chemistry. This vaccine design is analogous to other opioid vaccines that are being prepared for clinical use (Raleigh et al., PloS One 2017; 12:e0184876; Raleigh et al., The Journal of Pharmacology and Experimental Therapeutics 2018; 365:346-353). Mice and rats immunized with F-KLH had lower fentanyl-induced antinociception compared to controls. Rats immunized with F-KLH had lower brain fentanyl concentrations following an intravenous (i.v.) dose of fentanyl compared to controls. In a separate cohort of rats, F-sKLH reduced fentanyl-induced hotplate antinociception, respiratory depression, and bradycardia over a range of cumulative subcutaneous (s.c.) fentanyl doses. Together, these data suggest that a fentanyl vaccine could be a viable option for reducing the respiratory depressive effects as well as cardiac toxicity of fentanyl (and its synthetic analogs) in humans, and possibly prevent or reduce the likelihood of fatal overdoses upon accidental or deliberate intake of fentanyl, fentanyl analogs, fentanyl-laced drug mixtures, and fentanyl analog-laced drug mixtures.

As described in Example 2, vaccination with F₁ conjugated to CRM resulted in unexpectedly better results than F₁ conjugated to KLH or sKLH—including a higher fentanyl-specific IgG titer, greater efficacy against fentanyl- and sufentanil-induced antinociception, and decreased fentanyl-induced respiratory depression and bradycardia. In addition, immunization with F₁-CRM in rats undergoing fentanyl intravenous self-administration (FSA) reduced fentanyl intake during a FSA maintenance protocol and further prevented compensation during a dose reduction protocol compared to control rats immunized with CRM as control. Although it has been shown that vaccination can prevent acquisition of oxycodone intravenous self-administration (Pravetoni et al., PLOSone 2014; 9(7):e101807; Nguyen et al. Neuropharmacology, 2018; 134:57-64), it is not clear whether vaccinated rats could compensate during intravenous self-administration of oxycodone or other opioids. As described in Example 2, vaccination reduced fentanyl intake in rats with ongoing FSA and furthermore prevented the compensation in FSA observed in control rats. These data suggest that ongoing opioid use does not affect the immunogenicity and efficacy of the vaccine, and that that individuals vaccinated against fentanyl will likely not increase their fentanyl intake to overcome the effects of the vaccine. These data also suggest that, in contrast to opioid receptor antagonists such as naltrexone, an opioid detoxification regimen would not be required prior to initiation of immunotherapy with the vaccine in patients with ongoing use of fentanyl or another (that is, non-fentanyl) opioid.

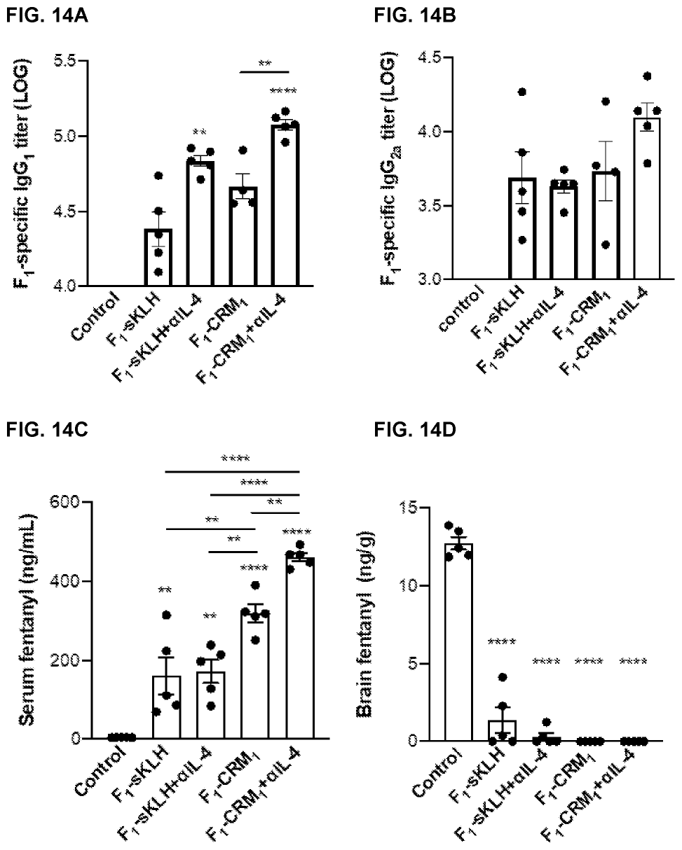

Surprisingly, as described in Example 2 and FIG. 14, the efficacy of an anti-fentanyl vaccine was enhanced by blockade of interleukin 4 (IL-4), and the effect of interleukin 4 (IL-4) was enhanced in mice immunized with F₁-CRM compared to F₁-sKLH. These results were particularly surprising because co-administration of α-IL-4 was previously shown to enhance the efficacy of an anti-oxycodone vaccine (Laudenbach et. al., Sci. Rep. 2018; 8(1):5508; WO 2017/196943 A1). In light of the previously published results, it was unexpected that anti-IL-4 would increase efficacy of any opioid vaccine, and it was not expected that the effect of blocking IL-4 may be dependent on the composition of the vaccine (for example, F₁-CRM vs F₁-sKLH).

### Fentanyl Hapten

In one aspect, this disclosure describes a composition including a fentanyl hapten. In some embodiments, the fentanyl hapten may include

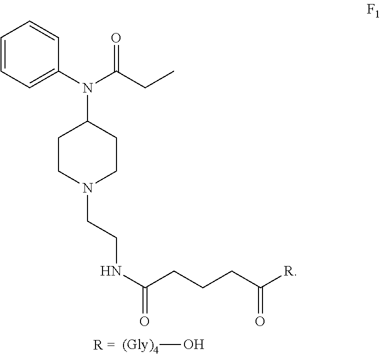

In some embodiments, the fentanyl hapten may consist essentially of F₁. In some embodiments, the fentanyl hapten may consist of F₁. Notably, F₁ is missing one of the rings present in fentanyl, sufentanil, and acetylfentanyl (see FIG. 1E).

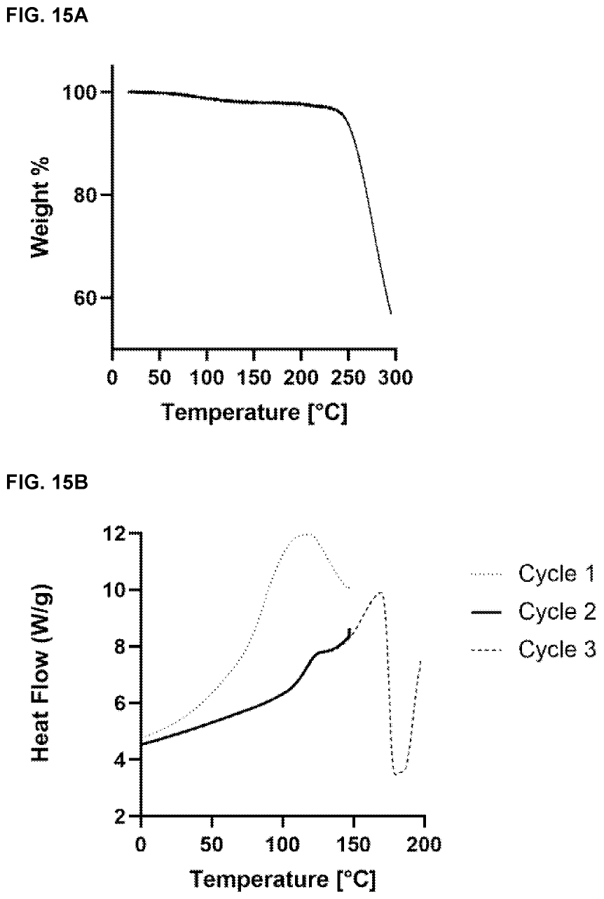

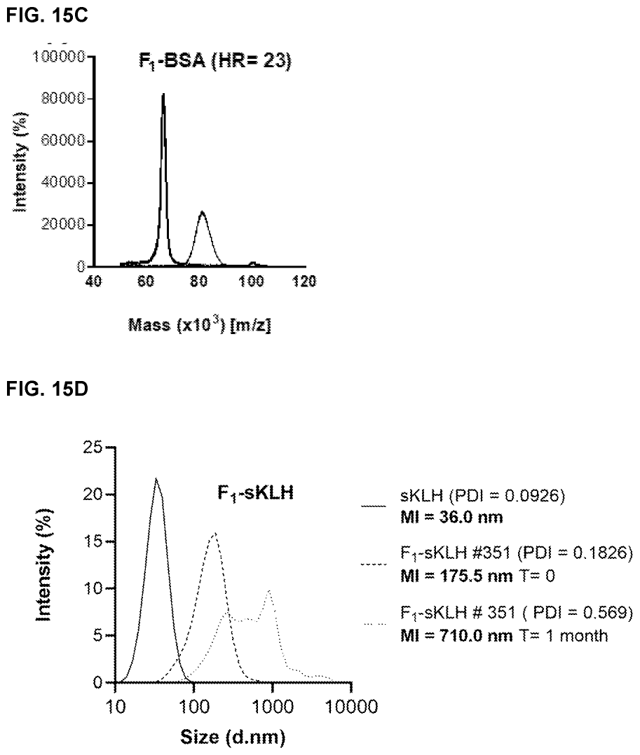

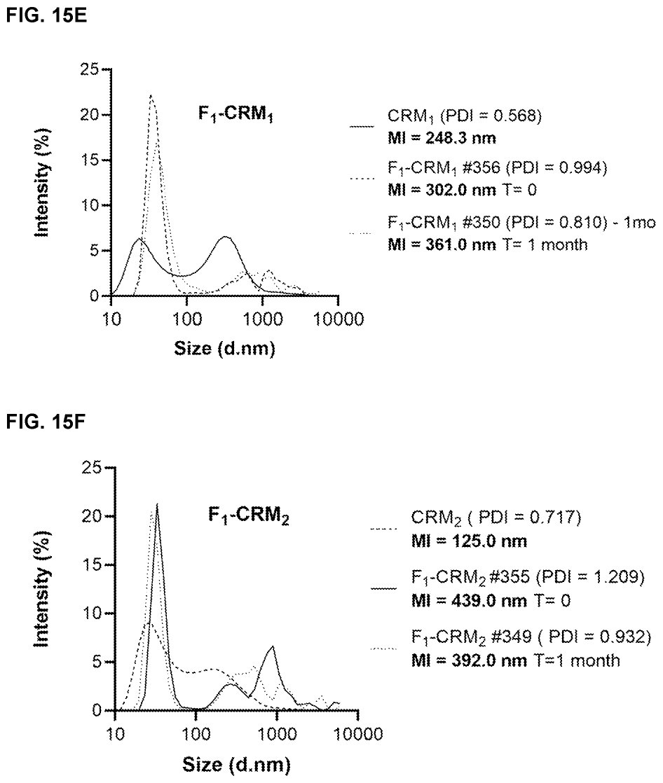

In some embodiments, the F₁ has a differential scanning calorimetry (DSC) thermogram exhibiting an endothermic event having a melt maxima temperature in a range of 110 degrees Celsius (° C.) to 130° C. or in a range of 115° C. to 125° C. In some embodiments, the F₁ has a differential scanning calorimetry (DSC) thermogram exhibiting an endothermic event having a melt maxima temperature in a range of 175° C. to 185° C. or in a range of 177° C. to 183° C. In some embodiments, F₁ has a differential scanning calorimetry (DSC) thermogram substantially the same as the DSC thermogram of FIG. 15B.

The skilled person will be aware that a DSC thermogram may be obtained which has one or more measurement errors depending on measurement conditions (such as equipment, sample preparation or instrument used). In particular, it is generally known that onset and/or peak temperatures may fluctuate depending on measurement conditions and sample preparation. Accordingly, it will be understood that the onset and/or peak temperature values of the DSC may vary slightly from one instrument to another, one method to another, from one sample preparation to another, and depending on the purity of the sample, and so the values quoted are not to be construed as absolute. As used herein, “substantially the same” when referring to a DSC thermogram means that the hapten provides a melt onset that is within ±5° C., and more preferably within ±1° C. of the value shown in the thermograms referenced herein.

In some embodiments, the F1 has a decompensation temperature of at least 200° C., at least 225° C., or at least 250° C., as measured by thermogravimetric analysis (TGA). In some embodiments, the F₁ has a TGA profile substantially the same as the TGA profile of FIG. 15A.

In some embodiments, the F₁ exhibits a haptenation ratio (number of hapten molecules per carrier molecule) to BSA, of at least 10, more preferably at least 15, even more preferably at least 20, and most preferably more than 20. In some embodiments, the haptenation ratio may be up to 25 or up to 30. For example, in an exemplary embodiment, the haptenation ratio may be in a range of 15 to 30 or in a range of 20 to 25. The haptenation ratio may be measured with mass spectrometry.

As described in Examples 1 and 2, F₁ made according to the synthesis methods of Example 1 exhibits a haptenation ratio to BSA of 8. In contrast, F₁ made according to the synthesis methods of Example 5 exhibits a haptenation ratio to BSA of 22.

Additionally, F₁ made according to the synthesis methods of Example 1 exhibited a dirty or gray appearance; in contrast, F₁ made according to the synthesis methods of Example 5 was whiter and more uniform.

### Fentanyl Hapten-Carrier Conjugate

In another aspect this disclosure describes a fentanyl hapten-carrier conjugate.

The fentanyl hapten-carrier conjugate includes a fentanyl-based hapten containing a tetraglycine linker (referred to herein as F, F₁, or F(Gly)₄), the structure of which is shown in FIG. 1B. As used herein, “fentanyl hapten” refers to molecule which, when combined with a carrier, can elicit the production of antibodies which bind to fentanyl or its analogs.

In the fentanyl hapten-carrier conjugate the fentanyl hapten F₁ is conjugated to an immunogenic carrier. Any suitable carrier may be used. In some embodiments, the immunogenic carrier includes, for example, bovine serum albumin (BSA), ovalbumin (OVA), keyhole limpet hemocyanin (KLH) including, for example, GMP grade subunit KLH (sKLH); diphtheria toxin, CRM, a genetically detoxified form of diphtheria toxin; tetanus toxin or tetanus toxoid (TT); pseudomonas exotoxin A; cholera toxin or toxoid; a Group A streptococcal toxin; a liposome; human gamma globulin; chicken immunoglobulin G; bovine gamma globulin; pneumolysin of Streptococcus pneumoniae; filamentous haemagglutinin (FHA); FHA fragments of Bordetella pertussis; pili or pilins of Neisseria gonorrhoeae; pili or pilins of Neisseria meningitidis; outer membrane proteins of Neisseria meningitidis, outer membrane proteins of Neisseria gonorrhoeae; C5A peptidase of Streptococcus; a surface protein of Moraxella catarrhalis; macro-, micro-, and nano-particles or combinations thereof (including synthetic macro-, micro-, and nano-particles); a carbon-based particle; a nanocarrier; a protein or peptide of viral, bacterial, or synthetic origin; or another immunogenic component; or a mixture or combination thereof.

In an exemplary embodiment, the fentanyl hapten-carrier conjugate includes F₁-KLH or F₁-Sklh. KLH may include the native decamer or di-decamer KLH form and sKLH may include a monomeric subunit or an homo- or heterogeneous dimeric assembly of subunits.

In some embodiments, the fentanyl hapten-carrier conjugate preferably includes F₁-CRM. The CRM may include E. coli-expressed CRM (EcoCRM) (available from Fina Biosolutions, Rockville, Md.) (also referred to herein as CRM₁) and/or CRM₁₉₇ (available from PFEnex, San Diego, Calif.) (also referred to herein as CRM₂).

An F₁-CRM may be preferred over, for example, a F₁-KLH conjugate for a variety of reasons. For example, as described in Example 2, immunization with F₁-CRM₁, and F₁-CRM₂ showed increased efficacy over the previously characterized F₁-sKLH. Further, as described in Example 3C, F₁-CRM₁, and F₁-CRM₂ were stable for at least 1 month. And, as shown in FIG. 15, F₁ conjugated to CRM results in conjugates with a smaller size and less aggregation than F₁-sKLH; this smaller conjugate is more suitable to sterile filtration using 0.45 nm and 0.22 nm filters, which may facilitate scale-up and manufacturing.

In some embodiments, the fentanyl hapten may be conjugated to a single immunogenic carrier via carbodiimide, maleimide, NETS-ester, or other coupling chemistry. In some embodiments, multiple F₁ may be conjugated to a single immunogenic carrier. BSA is typically used to optimize the conjugation reaction. For example, as described in Example 1, 8 molecules of F₁ hapten may be conjugated to one BSA, and as described in Example 2, 22 F₁ may be conjugated to one BSA. In some embodiments the protein haptenation ratio (number of hapten molecules per carrier molecule) is measured with mass spectrometry. In some embodiments, a higher haptenation ratio may enhance immunogenicity of the fentanyl hapten-carrier conjugate. Surprisingly, despite its low haptenation ratio (compared to conjugates containing nicotine, morphine, or oxycodone haptens) F₁-carrier conjugates described herein are highly effective.

In some embodiments, F₁ may be conjugated to a single immunogenic carrier along with other structurally distinct or structurally-related fentanyl-derived haptens to provide a multivalent display targeting multiple fentanyl analogs at once. In some embodiments, the fentanyl hapten-carrier conjugate including F₁ can be co-administered with other fentanyl hapten-carrier conjugates to provide a multivalent immunization strategy targeting multiple fentanyl analogs at once. In some embodiments, F₁ may be conjugated to a single immunogenic carrier along with other non-fentanyl haptens and/or unrelated opioid-haptens to provide a multivalent display targeting multiple opioids at once. Additionally or alternatively, in some embodiments, F₁ may be conjugated to a single immunogenic carrier along with a non-opioid drug-derived hapten to provide a multivalent display targeting an opioid (or multiple opioids) and a non-opioid drug at the same time. In some embodiments, the fentanyl hapten-carrier conjugate including F₁ can be co-administered with a non-fentanyl opioid and/or a non-opioid drug hapten-carrier conjugates to provide a multivalent immunization strategy targeting multiple opioids and/or non-opioid drugs at once.

### Compositions Including a Fentanyl Hapten-Carrier Conjugate

In some embodiments, this disclosure describes a composition including a fentanyl hapten-carrier conjugate described herein.

In some embodiments, the composition including the fentanyl hapten-carrier conjugate may further include an adjuvant or other delivery platform to augment the immunogenicity of the conjugate (for example, a particle, a bead, etc.). Any suitable adjuvant or delivery platform may be included. Exemplary adjuvants include, for example, an aluminum salt-based adjuvant (including, for example, aluminum hydroxide and aluminum phosphate), complete Freund's adjuvant (CFA), incomplete Freund's adjuvant (IFA), a phytol-based adjuvant, a carbohydrate-based adjuvant, a toll like receptor agonist (including, for example, monophosphoryl lipid A (MPLA) or a TLR ligand-based adjuvant), a oligomerization domain (NOD)-like receptor (NLR) agonist, a RIG-I-like receptor (RLR) agonist, a C-type lectin receptor (CLR) agonist, degradable nanoparticles (including, for example, poly-lactid-co-glycolid acid (PLGA)), or non-degradable nanoparticles (including, for example, latex, gold, silica or polystyrene), or combinations thereof.

Additionally or alternatively, suitable adjuvants include but are not limited to surfactants, for example, hexadecylamine, octadecylamine, lysolecithin, dimethyldioctadecylammonium bromide, N,N-dioctadecyl-N′—N-bis(2-hydroxyethyl-propane di-amine), methoxyhexadecyl-glycerol, and pluronic polyols; polanions, for example, pyran, dextran sulfate, poly IC, polyacrylic acid, carbopol; peptides, for example, muramyl dipeptide, aimethylglycine, tuftsin, oil emulsions, alum, and mixtures thereof. Other potential adjuvants include the B peptide subunits of E. coli heat labile toxin or of the cholera toxin, or CpG oligonucleotides.

In some embodiments, the adjuvant may preferably include aluminum (alum) salts.

In some embodiments, the composition may include a particular ratio of the fentanyl hapten-carrier conjugate to adjuvant. For example, in some embodiments, the fentanyl hapten-carrier conjugate:adjuvant ratio may be at least 1:3 or at least 2:3. In some embodiments, the fentanyl hapten-carrier conjugate:adjuvant ratio may be up to 2:3 or up to 3:3 (1:1).

In some embodiments, the composition may also include, for example, buffering agents to help to maintain the pH in an acceptable range or preservatives to retard microbial growth. A composition may also include, for example, pharmaceutically acceptable carriers, excipients, stabilizers, chelators, salts, or antimicrobial agents. Acceptable pharmaceutically acceptable carriers, excipients, stabilizers, chelators, salts, preservatives, buffering agents, or antimicrobial agents, include, but are not limited to, buffers such as phosphate, citrate, and other organic acids; antioxidants including ascorbic acid and methionine; preservatives, such as sodium azide, octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; polypeptides; proteins, such as serum albumin, gelatin, or non-specific immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zinc (Zn)-protein complexes); and/or non-ionic surfactants such as TWEEN, PLURONICS, or polyethylene glycol (PEG).

In some embodiments, the composition is a pharmaceutical composition and includes the fentanyl hapten-carrier conjugate and a pharmaceutically acceptable carrier, diluent or excipient. In the preparation of the pharmaceutical compositions including the fentanyl hapten-carrier conjugate described herein, a variety of vehicles and excipients may be used, as will be apparent to the skilled artisan.

The pharmaceutical compositions will generally include a pharmaceutically acceptable carrier and a pharmacologically effective amount of the fentanyl hapten-carrier conjugate, or mixture of fentanyl hapten-carrier conjugates.

The pharmaceutical composition may be formulated as a powder, a granule, a solution, a suspension, an aerosol, a solid, a pill, a tablet, a capsule, a gel, a topical cream, a suppository, a transdermal patch, and/or another formulation known in the art.

For the purposes described herein, pharmaceutically acceptable salts of a fentanyl hapten-carrier conjugate are intended to include any art-recognized pharmaceutically acceptable salts including organic and inorganic acids and/or bases. Examples of salts include but are not limited to sodium, potassium, lithium, ammonium, calcium, as well as primary, secondary, and tertiary amines, esters of lower hydrocarbons, such as methyl, ethyl, and propyl. Other salts include but are not limited to organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” includes any standard pharmaceutically accepted carriers known to those of ordinary skill in the art in formulating pharmaceutical compositions. For example, the fentanyl hapten-carrier conjugate may be prepared as a formulation in a pharmaceutically acceptable diluent, including for example, saline, phosphate buffer saline (PBS), aqueous ethanol, or solutions of glucose, mannitol, dextran, propylene glycol, oils (for example, vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or as a solid formulation in an appropriate excipient.

A pharmaceutical composition will often further include one or more buffers (for example, neutral buffered saline or phosphate buffered saline), carbohydrates (for example, glucose, sucrose or dextrans), mannitol, proteins, polypeptides or amino acids such as glycine, antioxidants (for example, ascorbic acid, sodium metabisulfite, butylated hydroxytoluene, butylated hydroxyanisole, etc.), bacteriostats, chelating agents such as EDTA or glutathione, adjuvants (for example, aluminum hydroxide), solutes that render the formulation isotonic, hypotonic or weakly hypertonic with the blood of a recipient, suspending agents, thickening agents and/or preservatives. Alternatively, compositions of the present disclosure may be formulated as a lyophilizate.

Any suitable carrier known to those of ordinary skill in the art may be employed in a composition including at least fentanyl hapten-carrier conjugate describes herein. Compositions including a fentanyl hapten-carrier conjugate may be formulated for any appropriate manner of administration, including for example, oral, nasal, mucosal, intravenous, intraperitoneal, intradermal, subcutaneous, and intramuscular administration.

### Methods of Making the Fentanyl Hapten-Carrier Conjugate and Composition Including the Fentanyl Hapten-Carrier Conjugate

The hapten of the fentanyl hapten-carrier conjugate may be synthesized by any suitable means. In some embodiments, F₁ of the fentanyl hapten-carrier conjugate may be synthesized as described in FIG. 1C and Example 1. In some embodiments, F₁ of the fentanyl hapten-carrier conjugate may be synthesized as described in FIG. 1D and Example 5.

In an exemplary embodiment, with reference to Scheme 2 (FIG. 1D), reductive amination of commercially available norfentanyl (1) may be conducted with N-Boc-2-aminoacetaldehyde in a solution of sodium triacetoxyborohydride and 1,2-dichloroethane to afford 2. Deprotection of 2 with trifluoroacetic acid in dichloromethane at room temperature may afford free amine 3. Acylation of 3 with methyl 5-chloro-5-oxopentanoate in the presence of triethylamine in dichloroethane may provide acylated product 4. In some embodiments, 4 may be purified using normal phase chromatography. In some embodiments, purification of 4 may be important to provide clean precursor 5 following a simple hydrolysis of the methyl ester with lithium hydroxide. Without such a purification step, preparation of intermediate 5 using glutaric anhydride may result in impure amino acid adducts that are extremely difficult to purify. Peptide coupling of 5 with tetraglycine methyl ester using (benzotriazol-1-yloxy)tris(dimethylamino)phosphonium hexafluorophosphate and triethylamine in dimethylformamide may provide intermediate 6. Base hydrolysis of 6 with lithium hydroxide in an equal mixture of water, tetrahydrofuran, and methanol may afford target hapten F1 in quantitative yield as a white solid.

In some embodiments, the identity and purity of the final product may be verified by mass spectrometry and/or NMR.

The fentanyl hapten may be conjugated to the carrier through any suitable means. In some embodiments, the fentanyl hapten may be conjugated to the carrier through coupling chemistry including, for example, through carbodiimide, maleimide, or NHS-ester chemistry. (See Pravetoni et al. Vaccine 2012; 30:4617-4624; Pravetoni et al., JPET 2012; 341:225-232; Baruffaldi et al. Mol Pharm. 2018; 15(11):4947-4962; Baruffaldi et al. Mol Pharm. 2019; 16(6):2364-2375; & Example 1.)

When the fentanyl hapten is conjugated to the carrier via carbodiimide (EDAC) chemistry, activation using N-ethyl-N′-(3 dimethylaminopropyl) carbodiimide hydrochloride may be useful. It may further be useful to prepare the hapten in a buffer including, for example IVIES. The buffer may include DMSO. Filtration of the couples conjugates may also be performed.

In an exemplary embodiment, conjugation of the F₁ hapten via carbodiimide (EDAC) chemistry may be performed as follows: the F₁ hapten may be dissolved at a concentration of 5.2 mM in 0.1 M MES buffer pH 4.5 containing 10% DMSO and may be activated by carbodiimide coupling chemistry using N-ethyl-N′-(3 dimethylaminopropyl) carbodiimide hydrochloride (EDAC, Sigma-Aldrich, St. Louis, Mo.) cross-linker at a final concentration of 208 mM. The mixture may be reacted for 10 minutes at room temperature (RT). BSA, sKLH, or CRM (or another carrier) may be added at a final concentration of 2.8 mg/mL, and the reactions were may be stirred for the following 3 hours at RT.

The final conjugates may be ultrafiltered (for example, using Amicon filters), with the filter size (for example, having a 50 kDa or a 100 kDa molecular cutoff) depending on the carrier protein dimensions. The final conjugates may be purified by tangential flow filtration (TFF) using membranes made of polyethersulfone (PES), cellulose, or the like. After having replaced IVIES buffer with phosphate-buffered saline (PBS) 0.1 M pH 7.2, the resulting solutions may be stored at 4° C.

In some embodiments, to avoid precipitation or aggregation, including, for example, when CRM is used as the carrier, a sugar may be included in the reacting buffer or storage buffer or both as a stabilizing agent. In an exemplary embodiment, the reacting buffer or storage buffer or both may include 250 mM sugar. In some embodiments, the sugar includes sucrose. Other sugars, including, for example, trialose or lactose, may be used to improve stability or storage conditions.

### Methods of Using the Fentanyl Hapten-Carrier Conjugate

In another aspect, this disclosure describes a method of using of a fentanyl hapten-carrier conjugate or a composition including the fentanyl hapten-carrier conjugate. In some embodiments, the fentanyl hapten-carrier conjugate or a composition including the fentanyl hapten-carrier conjugate may be used in an anti-opioid vaccine. In some embodiments, the fentanyl hapten-carrier conjugate or a composition including the fentanyl hapten-carrier conjugate may be used as a prophylactic vaccine to counteract toxicity from exposure to fentanyl and/or its analogues. In some embodiments, the fentanyl hapten-carrier conjugate or a composition including the fentanyl hapten-carrier conjugate may be used as a therapeutic vaccine to counteract toxicity from exposure to fentanyl and/or its analogues.

In some embodiments, the method includes administering the fentanyl hapten-carrier conjugate in combination with an opioid agonist or partial agonist. Exemplary opioid agonists or partial agonists include, for example, methadone, buprenorphine, etc. When the fentanyl hapten-carrier conjugate is administered in combination with an opioid agonist or partial agonist, the fentanyl hapten-carrier conjugate and the opioid agonist or partial agonist may be administered at the same time. It is also envisioned, however, that the fentanyl hapten-carrier conjugate may be administered days, weeks, months, or years in advance of an opioid agonist or partial agonist.

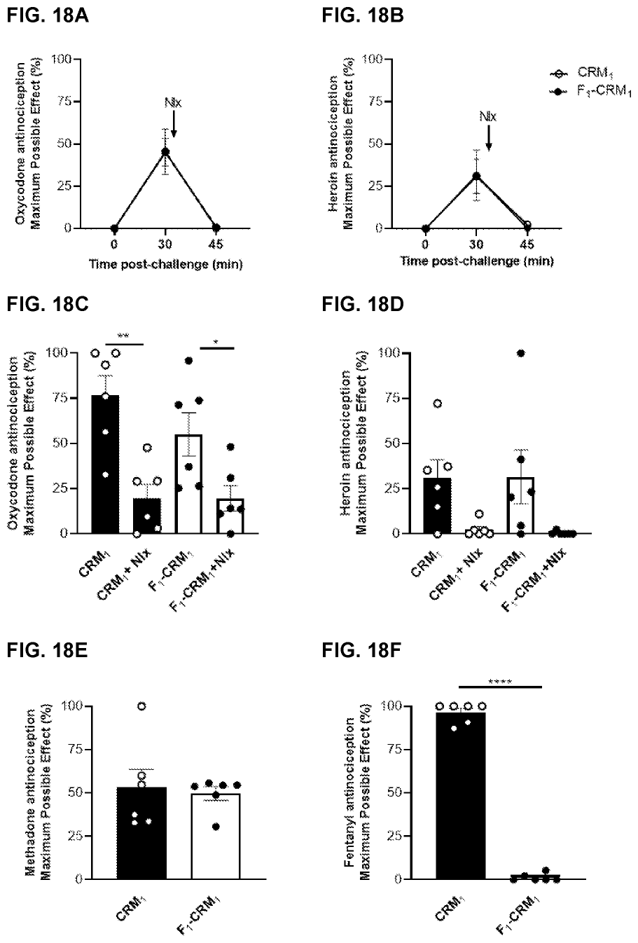

In some embodiments, the method includes administering the fentanyl hapten-carrier conjugate in combination with an opioid antagonist. Exemplary opioid antagonists include, for example, naloxone, nalmefene, naltrexone, etc. When the fentanyl hapten-carrier conjugate is administered in combination with an opioid antagonist, the fentanyl hapten-carrier conjugate and the opioid antagonist may be administered at the same time. It is also envisioned, however, that the fentanyl hapten-carrier conjugate may be administered days, weeks, months, or years in advance of an opioid antagonist. As described in Example 4 and FIG. 18, administration of a fentanyl hapten-carrier conjugate vaccine does not interfere with reversal of opioid overdose by administration of opioid antagonist such as naloxone.

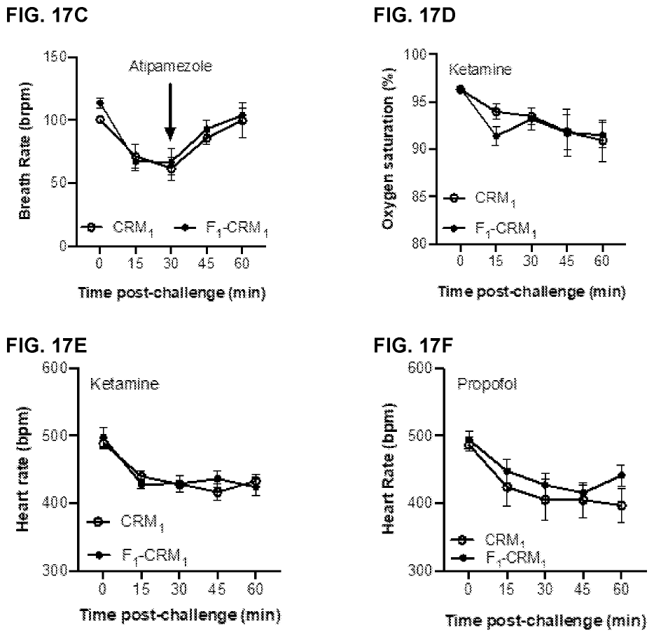

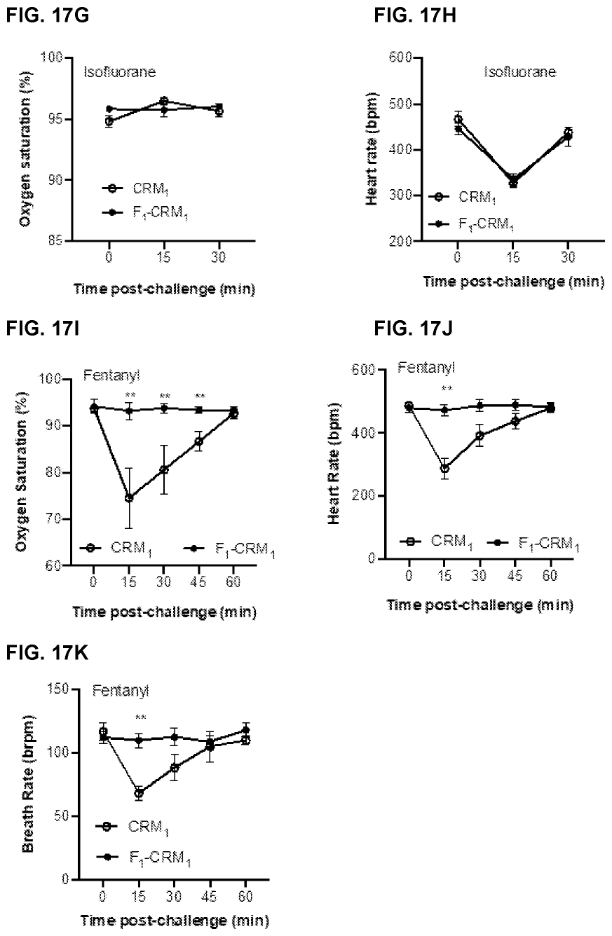

In some embodiments, the method includes administering the fentanyl hapten-carrier conjugate in combination with an anesthetic agent. When the fentanyl hapten-carrier conjugate is administered in combination with an anesthetic agent, the fentanyl hapten-carrier conjugate and the anesthetic agent may be administered at the same time. It is principally envisioned, however, that the fentanyl hapten-carrier conjugate may be administered days, weeks, months, or years in advance of the anesthetic agent. As shown in Example 4 and FIG. 17, administration of a fentanyl hapten-carrier conjugate vaccine does not interfere with pharmacological activity of commonly used anesthetic agents.

As described in Example 1, a fentanyl hapten-carrier conjugate including F₁ may provide an effective fentanyl vaccine. Specifically, F₁-sKLH reduced fentanyl-induced hotplate antinociception, respiratory depression, and bradycardia. The data in Example 1 suggest that a fentanyl vaccine could be a viable option for reducing the respiratory depressive effects and cardiac toxicity of fentanyl in humans, effects which are commonly associated with fatal overdoses. Moreover, even after treatment with a fentanyl hapten-carrier conjugate including F₁, naloxone reversed fentanyl effects, showing that naloxone's ability to reverse respiratory depression was preserved and that no cross-reactivity between anti-fentanyl antibodies generated by vaccination and naloxone existed. Lack of cross-reactivity (that is, selectivity) permits clinical use of vaccines in combination with current treatment for OUD and overdose. The data in Example 2 suggest that a fentanyl vaccine could be a viable option for patients with an ongoing fentanyl use disorder and that it would not be necessary to first detoxify patients (like in the case of naltrexone or other opioid antagonist) prior to the initiation of an immunization regimen. In addition, data in Example 2 suggest that the immunogenicity and efficacy of a fentanyl vaccine is not affected by concurrent fentanyl use, and that vaccination does not trigger withdrawal symptoms. Furthermore, in rats with ongoing fentanyl self-administration, vaccination with F₁-CRM did not induce compensation but rather decreased fentanyl intake suggesting that these vaccines are safe and will not trigger over-intake of fentanyl or other synthetic analog (for example, sufentanil or carfentanil) to overcome the effect of the vaccine.

Moreover, Example 4D suggests vaccination with F₁-CRM may also be effective against acetylfentanyl-induced respiratory depression and bradycardia during sequential challenges with acetylfentanyl doses. These results were surprising because it indicates that vaccination with F₁-CRM is not only protective against fentanyl but also fentanyl analogs (including, for example, sufentanil, acetylfentanyl, carfentanil, and/or other known or emerging (new) analogs). Specifically, it was unexpected that the F1 hapten—which is missing a key structural component of the fentanyl core (see FIG. 1E)—would be capable of inducing antibodies targeted against multiple analogs (for example, fentanyl, sufentanil, and acetylfentanyl) while preserving the selectivity against off target compounds such as anesthetics, opioid agonists and antagonists.

In some embodiments, including when the fentanyl hapten-carrier conjugate is used in an anti-opioid vaccine, a composition including the fentanyl hapten-carrier conjugate preferably includes an adjuvant or other immunostimulatory molecule.

The fentanyl hapten-carrier conjugate may be administered to any subject determined to benefit. For example, in some embodiments, administration of an anti-opioid vaccine may be used to treat a subject who may be exposed to an opioid or that has been exposed to an opioid or that is suspected of having been exposed to an opioid. Exemplary opioids include fentanyl and fentanyl analogs (including, for example, sufentanil, acetylfentanyl, or carfentanil). Surprisingly, the fentanyl hapten-carrier conjugate induces antibodies that react not only with fentanyl, but also with its analogs. This result is particularly surprising because F₁ is missing one of the rings present in fentanyl, sufentanil, and acetylfentanyl (FIG. 1E), yet the fentanyl hapten (F₁)-carrier conjugate may act as a vaccine against each of these compounds while preserving selectivity (that is, not binding to methadone, heroin, oxycodone, naloxone, anesthetics, etc).

In some embodiments, a subject may include, for example, a soldier, a law enforcement professional, a health profession, a first responder, etc. In some embodiments, a subject may include an individual who has been diagnosed with an opioid use disorder including, in some cases, an individual who has recovered from an opioid use disorder. In some embodiments, a subject may include an individual who has been diagnosed with a substance use disorder including, in some cases, an individual who has recovered from a substance use disorder. In some embodiments, a subject may include a pregnant mother treated for an opioid use disorder or a substance use disorder. In some embodiments, a subject may include a newborn child of a mother treated for an opioid use disorder or a substance use disorder. In some embodiments, a subject may include a pregnant mother being treated for an opioid use disorder or a substance use disorder. In some embodiments, a subject may include a patient currently treated with methadone or buprenorphine for treatment of an opioid use disorder. In some embodiments, a subject may include a patient currently treated with prescription opioids, including, for example, oxycodone, for treatment of acute or chronic pain. In some embodiments, a subject may include a patient that is planning to undergo surgery or another critical care medical procedure that requires anesthetic.

A composition including a fentanyl hapten-carrier conjugate of the present disclosure may be formulated in pharmaceutical preparations in a variety of forms adapted to the chosen route of administration. One of skill will understand that the composition will vary depending on mode of administration and dosage unit. For example, for parenteral administration, isotonic saline may be used. Other suitable carriers include, but are not limited to alcohol, phosphate buffered saline, and other balanced salt solutions. The compounds of this invention may be administered in a variety of ways, including, but not limited to, intravenous, topical, oral, subcutaneous, intraperitoneal, and intramuscular delivery. In some aspects, the compounds of the present disclosure may be formulated for controlled or sustained release. In some aspects, a formulation for controlled or sustained release is suitable for oral implantation. In some aspects, a formulation for controlled or sustained release is suitable for subcutaneous implantation. Any suitable means of achieving controlled or sustained release may be used including, for example, embedding the fentanyl hapten-carrier conjugate in a matrix of insoluble substance, the use of a reservoir device or a matrix device, cross-liking the fentanyl hapten-carrier conjugate to an ion exchange resin, etc. In some aspects, a formulation for controlled or sustained release includes a patch. A compound may be formulated for enteral administration, for example, formulated as a capsule or tablet.

Administration may be as a single dose or in multiple doses. In some embodiments, the dose is an effective amount as determined by the standard methods, including, but not limited to, those described herein. Those skilled in the art of clinical trials will be able to optimize dosages of particular compounds through standard studies. Additionally, proper dosages of the compositions may be determined without undue experimentation using standard dose-response protocols. Administration includes, but is not limited to, any of the dosages and dosing schedules, dosing intervals, and/or dosing patterns described in the examples included herewith.

The composition including a fentanyl hapten-carrier conjugate according to the present disclosure may be administered by any suitable means including, but not limited to, for example, oral, rectal, nasal, topical (including transdermal, aerosol, buccal and/or sublingual), vaginal, parenteral (including subcutaneous, intramuscular, and/or intravenous), intradermal, intravesical, intra-joint, intra-arteriole, intraventricular, intracranial, intraperitoneal, intranasal, or by inhalation.

For use as a vaccine, the composition including a fentanyl hapten-carrier conjugate may be administered parenterally, for example, by intramuscular, intradermal, or subcutaneous injection. Other modes of administration, including, for example mucosal administration, are also envisioned.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that may be employed will be known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Such preparations may be pyrogen-free.

Many suitable formulations are known, including polymeric or protein microparticles encapsulating drug to be released, ointments, gels, or solutions which may be used topically or locally to administer drug, and even patches, which provide controlled release over a prolonged period of time. These may also take the form of implants.

The compounds may also be provided in a lyophilized form. Such compositions may include a buffer, for example, bicarbonate, for reconstitution prior to administration, or the buffer may be included in the lyophilized composition for reconstitution with, for example, water. The lyophilized composition may further include a suitable vasoconstrictor, for example, epinephrine. The lyophilized composition may be provided in a syringe, optionally packaged in combination with the buffer for reconstitution, such that the reconstituted composition may be immediately administered to a patient.

In some embodiments, for example, a composition including a fentanyl hapten-carrier conjugate according to the present disclosure may be given before a subject is exposed to an opioid to prevent or mitigate the effects of opioid exposure. Additionally or alternatively, a composition including a fentanyl hapten-carrier conjugate according to the present disclosure may be given after a subject is exposed to an opioid to reverse or mitigate the effects of a subsequent opioid exposure. Additionally or alternatively, a composition including a fentanyl hapten-carrier conjugate according to the present disclosure may be given after a subject is exposed to an opioid to prevent or reduce likelihood of fatal overdose. Additionally or alternatively, a composition including a fentanyl hapten-carrier conjugate according to the present disclosure may be given as prophylaxis measure to those at risk of mass casualty incidents or chemical attacks, or other form of deliberate poisoning.

Effective concentrations and amounts may be determined for each application herein empirically by testing the compounds in known in vitro and in vivo systems, such as those described herein, dosages for humans or other animals may then be extrapolated therefrom.

A composition as described herein may be administered at once, or may be divided into a number of smaller doses to be administered at intervals of time. For example, compositions may be administered repeatedly, for example, at least 2, 3, 4, 5, 6, 7, 8, or more times, or may be administered by continuous infusion. It is understood that the precise dosage and duration of treatment may be determined empirically using known testing protocols or by extrapolation from in vivo or in vitro test data. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the recited compositions and methods.

In some therapeutic embodiments, an “effective amount” of an agent (for example, a fentanyl hapten-carrier conjugate) is an amount that results in a reduction of at least one pathological parameter upon exposure to an opioid. Exemplary parameters include respiratory depression and bradycardia. Thus, for example, in some aspects, an effective amount is an amount that is effective to achieve a reduction of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% compared to the expected reduction in the parameter in an individual not treated with the agent.

In some aspects, the administration of a fentanyl hapten-carrier conjugate may allow for the effectiveness of a lower dosage of other therapeutic modalities when compared to the administration of the other therapeutic modalities alone, providing relief from the toxicity observed with the administration of higher doses of the other modalities. For example, in some aspects, pre-administration of a fentanyl hapten-carrier conjugate vaccine may be used to decrease the amount of naloxone, nalmefene, or an anti-opioid antibody that would otherwise be needed to protect a patient. Furthermore, as shown in Example 4 and FIG. 18, administration of a fentanyl hapten-carrier conjugate vaccine does not interfere with reversal of opioid overdose (with, for example, an opioid antagonist such as naloxone).

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Fentanyl Hapten Aspects

A1. A fentanyl hapten comprising

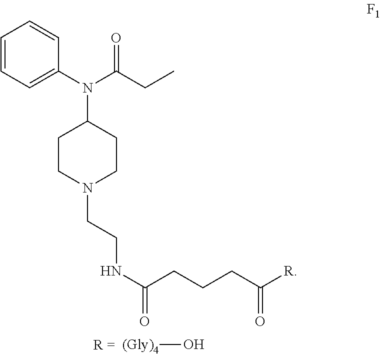

A2. The fentanyl hapten of Aspect A1,

wherein the F₁ has a differential scanning calorimetry (DSC) thermogram exhibiting an endothermic event having a melt maxima temperature in a range of 110 degrees Celsius (° C.) to 130° C.;

wherein the F₁ has a DSC thermogram exhibiting an endothermic event having a melt maxima temperature in a range of 175° C. to 185° C.;

wherein the F1 has a decompensation temperature at least 200° C., at least 225° C., or at least 250° C., as measured by thermogravimetric analysis (TGA); or

wherein the F1 has a haptenation ratio to BSA, of at least 10, at least 15, at least 20; or more than 20, or

a combination thereof.

A3. A fentanyl hapten consisting essentially of

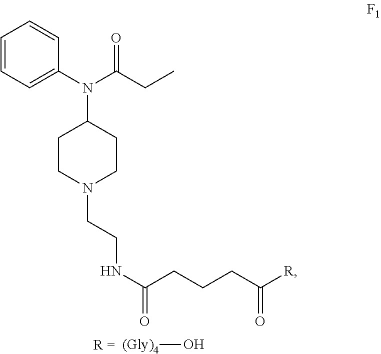

wherein the F₁ has a differential scanning calorimetry (DSC) thermogram exhibiting an endothermic event having a melt maxima temperature in a range of 110 degrees Celsius (° C.) to 130° C.;

wherein the F₁ has a DSC thermogram exhibiting an endothermic event having a melt maxima temperature in a range of 175° C. to 185° C.;

wherein the F1 has a decompensation temperature of at least 200° C., at least 225° C., or at least 250° C., as measured by thermogravimetric analysis (TGA); or

wherein the F1 has a haptenation ratio to BSA, of at least 10, at least 15, at least 20; or more than 20; or

a combination thereof.

A4. A fentanyl hapten consisting of

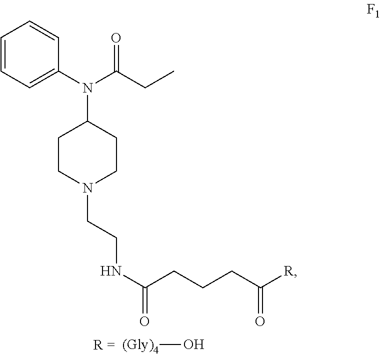

wherein the F₁ has a differential scanning calorimetry (DSC) thermogram exhibiting an endothermic event having a melt maxima temperature in a range of 110 degrees Celsius (° C.) to 130° C.;

wherein the F₁ has a DSC thermogram exhibiting an endothermic event having a melt maxima temperature in a range of 175° C. to 185° C.;

wherein the F1 has a decompensation temperature of at least 200° C., at least 225° C., or at least 250° C., as measured by thermogravimetric analysis (TGA); or

wherein the F1 has a haptenation ratio to BSA, of at least 10, at least 15, at least 20; or more than 20; or

a combination thereof.

A5. The fentanyl hapten of any one of Aspects A1 to A4,

wherein the F₁ has a differential scanning calorimetry (DSC) thermogram exhibiting an endothermic event having a melt maxima temperature in a range of 110 degrees Celsius (° C.) to 130° C.;

wherein the F₁ has a DSC thermogram exhibiting an endothermic event having a melt maxima temperature in a range of 175° C. to 185° C.;

wherein the F1 has a decompensation temperature of at least 200° C., at least 225° C., or at least 250° C., as measured by thermogravimetric analysis (TGA); and

wherein the F1 has a haptenation ratio to BSA, of at least 10, at least 15, at least 20; or more than 20.

A6. The fentanyl hapten of any one of Aspects A1 to A5, wherein the F₁ exhibits a differential scanning calorimetry (DSC) thermogram substantially the same as the DSC thermogram of FIG. 15B.
A7. The fentanyl hapten of any one of Aspects A1 to A6, wherein the F₁ has a TGA profile substantially the same as the TGA profile of FIG. 15A.
A8. The fentanyl hapten of any one of Aspects A1 to A7, wherein the F₁ has a haptenation ratio to BSA of at least 20.
A9. The fentanyl hapten of any one of Aspects A1 to A8, wherein the F₁ has a haptenation ratio to BSA of 22.

### Exemplary Fentanyl Hapten-Carrier Conjugate Aspects

B1. A fentanyl hapten-carrier conjugate comprising

a fentanyl hapten comprising

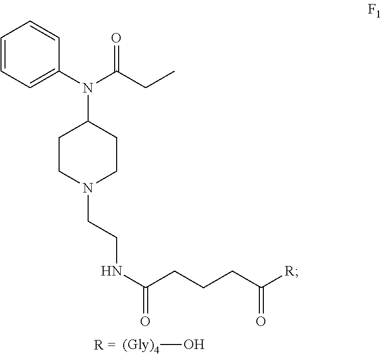

and

an immunogenic carrier, wherein the fentanyl hapten is conjugated to the immunogenic carrier.

B2. The fentanyl hapten-carrier conjugate of Aspect B1, wherein the immunogenic carrier comprises a carrier selected from bovine serum albumin (BSA), ovalbumin (OVA), keyhole limpet hemocyanin (KLH); CRM; a liposome, tetanus toxoid (TT); a peptide; macro-, micro-, and nano-particles or combinations thereof; a carbon-based particle; a nanocarrier; a protein of viral, bacterial, or synthetic origin; or another immunogenic component; or a mixture or combination thereof.
B3. The fentanyl hapten-carrier conjugate of Aspect B1 or B2, wherein the immunogenic carrier comprises KLH.
B4. The fentanyl hapten-carrier conjugate of any one of Aspects B1 to B3, wherein the immunogenic carrier comprises GMP grade subunit KLH (sKLH).
B5. The fentanyl hapten-carrier conjugate of any one of Aspects B1 to B4, wherein the immunogenic carrier comprises CRM.
B6. The fentanyl hapten-carrier conjugate of any one of Aspects B1 to B5,

wherein the F₁ has a differential scanning calorimetry (DSC) thermogram exhibiting an endothermic event having a melt maxima temperature in a range of 110 degrees Celsius (° C.) to 130° C.;

wherein the F₁ has a DSC thermogram exhibiting an endothermic event having a melt maxima temperature in a range of 175° C. to 185° C.;

wherein the F1 has a decompensation temperature of at least 200° C., at least 225° C., or at least 250° C., as measured by thermogravimetric analysis (TGA); or

wherein the F1 has a haptenation ratio to BSA, of at least 10, at least 15, at least 20; or more than 20; or

a combination thereof.

B7. The fentanyl hapten-carrier conjugate of any one of Aspects B1 to B6, wherein

the F₁ has a differential scanning calorimetry (DSC) thermogram exhibiting an endothermic event having a melt maxima temperature in a range of 110 degrees Celsius (° C.) to 130° C.;

wherein the F₁ has a DSC thermogram exhibiting an endothermic event having a melt maxima temperature in a range of 175° C. to 185° C.;

wherein the F1 has a decompensation temperature of at least 200° C., at least 225° C., or at least 250° C., as measured by thermogravimetric analysis (TGA); and

wherein the F1 has a haptenation ratio to BSA, of at least 10, at least 15, at least 20; or more than 20.

B8. The fentanyl hapten-carrier conjugate of any one of Aspects B1 to B7, wherein the F₁ exhibits a differential scanning calorimetry (DSC) thermogram substantially the same as the DSC thermogram of FIG. 15B.
B9. The fentanyl hapten-carrier conjugate of any one of Aspects B1 to B8, wherein the F₁ has a TGA profile substantially the same as the TGA profile of FIG. 15A.
B10. The fentanyl hapten-carrier conjugate of any one of Aspects B1 to B9, wherein the F₁ has a haptenation ratio to BSA of at least 10, at least 15, at least 20; or more than 20.
B11. The fentanyl hapten-carrier conjugate of any one of Aspects B1 to B10, wherein the F₁ has a haptenation ratio to BSA of 22.
B12. The fentanyl hapten-carrier conjugate of any one of Aspects B1 to B11, wherein the fentanyl hapten is conjugated to the immunogenic carrier through coupling chemistry.
B13. The fentanyl hapten-carrier conjugate of any one of Aspects B1 to B12, wherein the fentanyl hapten is conjugated to the immunogenic carrier through carbodiimide chemistry.

### Exemplary Composition Aspects

C1. A composition comprising the fentanyl hapten-carrier conjugate of any one of Aspects B1 to B13.
C2. The composition of Aspect C1, wherein the composition further comprises an adjuvant.
C3. The composition of Aspect C2, wherein the adjuvant comprises an aluminum salt based adjuvant, complete Freund's adjuvant (CFA), incomplete Freund's adjuvant (IFA), a phytol-based adjuvant, a carbohydrate-based adjuvant, a toll like receptor agonist, a oligomerization domain (NOD)-like receptor (NLR) agonist, a RIG-I-like receptor (RLR) agonist, a C-type lectin receptor (CLR) agonist, degradable nanoparticles, or non-degradable nanoparticles, or combinations thereof.

### Exemplary Method of Making Aspects

D1. A method of making the fentanyl hapten of any one of the Exemplary Fentanyl Hapten Aspects (A1 to A9).
D2. The method of Aspect D1, the method comprising using the synthesis described in Scheme 1 (FIG. 1C).
D3. The method of Aspect D1, the method comprising using the synthesis described in Scheme 2 (FIG. 1D) and/or Example 5.
D4. The method of Aspect D3, wherein

step a of Scheme 2 comprises using Na(OAc)₃BH and dichloroethane (DCE);

step b of Scheme 2 comprises using trifluoroacetic acid (TFA) in dichloromethane (DCM);

step c of Scheme 2 comprises using methyl 5-chloro-5-oxopentanoate in the presence of triethylamine (TEA) in dichloroethane (DCM);

step d of Scheme 2 comprises using lithium hydroxide (LiOH);

step e of Scheme 2 comprises using tetraglycine methyl ester, (benzotriazol-1-yloxy)tris(dimethylamino)phosphonium hexafluorophosphate (BOP), and dimethylformamide (DMF); and/or

step f of Scheme 2 comprises using lithium hydroxide (LiOH) in a mixture of tetrahydrofuran, methanol, and water (THF/MeOH/H₂O).

D5. The method of Aspect D4, wherein step d comprises using lithium hydroxide (LiOH) in a mixture of tetrahydrofuran, methanol, and water (THF/MeOH/H₂O).
D6. The method of Aspect D4 or D5, wherein the mixture of tetrahydrofuran, methanol, and water (THF/MeOH/H₂O) comprises a ratio of (1:1:0.5, v/v/v) parts tetrahydrofuran, methanol, and water.
D7. The method of any one of aspects D3 to D6, wherein

step a of Scheme 2 comprises using N-Boc-2-aminoacetaldehyde in a solution of sodium triacetoxyborohydride and 1,2-dichloroethane;

step b of Scheme 2 comprises using trifluoroacetic acid in dichloromethane at room temperature;

step c of Scheme 2 comprises using methyl 5-chloro-5-oxopentanoate in the presence of triethylamine in dichloroethane;

step d of Scheme 2 comprises purifying 4 using normal phase chromatography;

step e of Scheme 2 comprises using (benzotriazol-1-yloxy)tris(dimethylamino)phosphonium hexafluorophosphate and triethylamine in dimethylformamide; and/or

step f of Scheme 2 comprises using lithium hydroxide in mixture comprising tetrahydrofuran, methanol, and water (THF/MeOH/H₂O) in a ratio of (1:1:0.5, v/v/v).

D8. The method of any one of Aspects D1 to D7, wherein the method comprises conjugating the fentanyl hapten to an immunogenic carrier through coupling chemistry.
D9. A method of making the fentanyl hapten-carrier conjugate of any one of the Exemplary Fentanyl Hapten-Carrier Conjugate Aspects (B1 to B13).
D10. The method of Aspect D9, the method comprising using the synthesis described in Scheme 1 (FIG. 1C).
D11. The method of Aspect D6, the method comprising using the synthesis described in Scheme 2 (FIG. 1D) and/or Example 5.
D12. The method of Aspect D11, wherein

step a of Scheme 2 comprises using Na(OAc)₃BH and dichloroethane (DCE);

step b of Scheme 2 comprises using trifluoroacetic acid (TFA) in dichloromethane (DCM);

step c of Scheme 2 comprises using methyl 5-chloro-5-oxopentanoate in the presence of triethylamine (TEA) in dichloroethane (DCM);

step d of Scheme 2 comprises using lithium hydroxide (LiOH);

step e of Scheme 2 comprises using tetraglycine methyl ester, (benzotriazol-1-yloxy)tris(dimethylamino)phosphonium hexafluorophosphate (BOP), and dimethylformamide (DMF); and/or

step f of Scheme 2 comprises using lithium hydroxide (LiOH) in a mixture of tetrahydrofuran, methanol, and water (THF/MeOH/H₂O).

D13. The method of Aspect D12, wherein step d comprises using lithium hydroxide (LiOH) in a mixture of tetrahydrofuran, methanol, and water (THF/MeOH/H₂O).
D14. The method of Aspect D12 or D13, wherein the mixture of tetrahydrofuran, methanol, and water (THF/MeOH/H₂O) comprises a ratio of (1:1:0.5, v/v/v) parts tetrahydrofuran, methanol, and water.
D15. The method of any one of aspects D11 to D14, wherein

step a of Scheme 2 comprises using N-Boc-2-aminoacetaldehyde in a solution of sodium triacetoxyborohydride and 1,2-dichloroethane;

step b of Scheme 2 comprises using trifluoroacetic acid in dichloromethane at room temperature;

step c of Scheme 2 comprises using methyl 5-chloro-5-oxopentanoate in the presence of triethylamine in dichloroethane;

step d of Scheme 2 comprises purifying 4 using normal phase chromatography;

step e of Scheme 2 comprises using (benzotriazol-1-yloxy)tris(dimethylamino)phosphonium hexafluorophosphate and triethylamine in dimethylformamide; and/or step f of Scheme 2 comprises using lithium hydroxide in mixture comprising tetrahydrofuran, methanol, and water (THF/MeOH/H₂O) in a ratio of (1:1:0.5, v/v/v).

D16. The method of any one of Aspects D11 to D15, wherein the method comprises conjugating the fentanyl hapten to the immunogenic carrier through coupling chemistry.
D17. A method of making the composition of any one of Aspects C1 to C3.

### Exemplary Method of Using Aspects

E1. A method comprising administering the fentanyl hapten-carrier conjugate of any one of the Exemplary Fentanyl Hapten-Carrier Conjugate Aspects (B1 to B13) or the composition of any one of the Exemplary Composition Aspects (C1 to C3) to a subject.
E2. The method of Aspect E1, wherein the subject comprises a soldier, a law enforcement professional, a health profession, or a first responder.
E3. The method of Aspect E1 or E2, wherein the subject comprises an individual who has been diagnosed with an opioid use disorder and/or who has recovered from an opioid use disorder.
E4. The method of any one of Aspects E1 to E3, wherein the subject comprises an individual who has been diagnosed with a substance use disorder and/or who has recovered from a substance use disorder.
E5. The method of any one of Aspects E1 to E4, wherein the method comprises administering multiple doses of the fentanyl hapten-carrier conjugate or the composition comprising the fentanyl hapten-carrier conjugate to the subject.
E6. The method of any one of Aspects E1 to E5, wherein the method comprises administering the fentanyl hapten-carrier conjugate to the subject in combination with an opioid agonist or partial agonist.
E7. The method of any one of Aspects E1 to E6, wherein the method comprises administering the fentanyl hapten-carrier conjugate to the subject in combination with an opioid antagonist.
E8. The method of Aspect E7, wherein opioid antagonist comprises naloxone.
E9. The method of any one of Aspects E1 to E6, wherein the method comprises administering the fentanyl hapten-carrier conjugate to the subject in combination with an anesthetic agent.
E10. The method of any one of Aspects E1 to E6, wherein the method comprises administering the fentanyl hapten-carrier conjugate to the subject in combination with a non-fentanyl opioid or a non-opioid drug hapten-carrier conjugate.
E11. The method of any one of Aspects E1 to E10, wherein the subject may be exposed to an opioid.
E12. The method of Aspect E11, wherein the opioid comprises fentanyl.
E13. The method of Aspect E11 or E12, wherein the opioid comprises sufentanil, acetylfentanyl, and/or carfentanil.
E14. The method of any one of Aspects E1 to E13, wherein the subject has been exposed to an opioid or is suspected of having been exposed to an opioid.
E15. The method of Aspect E14, wherein the opioid comprises fentanyl.
E16. The method of Aspect E14 or E15, wherein the opioid comprises sufentanil, acetylfentanyl, and/or carfentanil.

The present invention is illustrated by the following examples. It is to be understood that the particular examples, materials, amounts, and procedures are to be interpreted broadly in accordance with the scope and spirit of the invention as set forth herein.

### EXAMPLES

### Example 1—A Fentanyl Vaccine Alters Fentanyl Distribution and Protects Against Fentanyl-Induced Effects in Mice and Rats

This Example (published as Raleigh et al. “A Fentanyl Vaccine Alters Fentanyl Distribution and Protects against Fentanyl-Induced Effects in Mice and Rats,” J. Pharmacol. Exp. Ther. 2019; 368:282-291) describes the development of a vaccine including a fentanyl hapten (F, also referred to herein as F₁ or F(Gly)₄,) conjugated to keyhole limpet hemocyanin (KLH) carrier protein or to GMP-grade subunit KLH (sKLH) by means of a tetraglycine linker using carbodiimide chemistry. Immunization with F-KLH in mice and rats reduced fentanyl-induced hotplate antinociception and in rats reduced fentanyl distribution to brain compared to controls. F-KLH did not reduce antinociceptive effects of equianalgesic doses of heroin or oxycodone in rats. To assess vaccine effect on fentanyl toxicity, rats immunized with F-sKLH or unconjugated sKLH were exposed to increasing s.c. doses of fentanyl. Vaccination with F-sKLH shifted the dose-response curves to the right for both fentanyl-induced antinociception and respiratory depression. Naloxone reversed fentanyl effects in both groups, showing that its ability to reverse respiratory depression was preserved. These data demonstrate pre-clinical selectivity and efficacy of a fentanyl vaccine and suggest that vaccines may offer a therapeutic option in reducing fentanyl-induced side effects including, for example, for reducing the respiratory depressive effects of fentanyl in humans.

### Materials and Methods

Animal Experiments. Surgery was performed under ketamine (75 mg/kg) and dexmedetomidine (0.05 mg/kg) anesthesia, animals were euthanized by CO2 inhalation using Association for Assessment and Accreditation of Laboratory Animal Care International—approved chambers, and all efforts were made to minimize suffering.

Overview of fentanyl hapten synthesis. Numbers in square brackets refer to the corresponding structure in FIG. 1C (Scheme 1).

The F hapten (also referred to herein as F₁ or F(Gly)₄, [8]) was synthesized as depicted in Scheme 1. Briefly, piperidone monohydrate hydrochloride [1] propanamide was alkylated with 2-(Bocamino)ethylbromide in the presence of potassium carbonate in acetonitrile to provide the N-substituted piperidine intermediate [2] with good yield. Reductive amination with aniline of piperidine intermediate [2] mediated by sodium cyanoborohydride in the presence of an equimolar amount of acetic acid yielded the 4-piperidineamine precursor [3] in excellent yield (91%). Then, the 4-piperidineamine precursor [3] was acylated using propionyl chloride in the presence of Hunig's base [N,N-diisopropylethylamine (DIPEA)] to provide compound [4]. Acid-mediated N-Boc terminal group deprotection followed by acylation with glutaric anhydride in presence of pyridine led to carboxylic acid [6]. Neither step required any further purification. The linker (Gly)₄-OtBu (Pravetoni et al., The Journal of Pharmacology and Experimental Therapeutics 2012; 341:225-232) was attached in classic fashion using 2-(1H-benzotriazol-1-yl)-1,1,3,3-tetramethyluronium hexafluorophosphate (HBTU) and DIPEA as coupling agents. Lastly, the tert-butyl ester [7] was hydrolyzed using 20% of trifluoracetic acid in dichloromethane to afford the hapten [8].

Fentanyl hapten. All commercial reagents and anhydrous solvents were used without further purification or distillation. Analytical thin-layer chromatography (TLC) was performed on a MilliporeSigma (Burlington, Mass.) silica gel 60 F254 coated plate (0.25 mm). Plates were visualized by UV light, iodine vapor, or ninhydrin solution. Flash column chromatography was performed on Thermo Fisher Scientific (Waltham, Mass.) silica gel (230-400 mesh) unless otherwise noted. Nuclear magnetic resonance CH NMR (400 MHz), ¹³C NMR (100 MHz)) spectra were determined on a Bruker (Billerca, Mass.) instrument unless otherwise noted. Chemical shifts for ¹H NMR are reported in parts per million (ppm) relative to chloroform (7.26 ppm) and coupling constants are in hertz (Hz). Chemical shifts are expressed in ppm and coupling constants (J) are in hertz (Hz). Peak multiplicities are abbreviated: broad, br; singlet, s; doublet, d; triplet, t; and multiplet, m. Chemical shifts for ¹³C NMR were reported in ppm relative to the center line of a triplet at 77.0 ppm for chloroform. Electrospray ionization (ESI) mode mass spectra were recorded on a BrukerBioTOF II mass spectrometer (Bruker, Billerca, Mass.), and the data were consistent with the considered structures. Elemental analyses for the target compound were performed by M-H-W Laboratories (Phoenix, Ariz.). Analytical data confirmed the purity of the products was ≥95%.

tert-butyl (2-(4-oxopiperidin-1-yl)ethyl)carbamate [2]. 4-piperidone monohydrate hydrochloride ([1] 2.0 g, 13 mmol) was dissolved in acetonitrile (40 mL) in a 250 mL round-bottom flask equipped with a large stir bar and a condenser. The colorless solution was treated sequentially with potassium carbonate (K₂CO₃, 3.26 g, 23.6 mmol) and 2-(Boc-amino)ethylbromide (1.96 mL, 11.8 mmol) at ambient temperature. The resulting suspension was vigorously stirred and refluxed at 80° C. for 8 hours. After 8 hours, the mixture was cooled to ambient temperature, transferred to a separatory funnel and partitioned (CH₂Cl₂//H₂O). The organic phase was washed with brine, saturated NaHCO₃(2×100 mL), dried over Na₂SO₄ and concentrated in vacuo to give a yellow oil. The oily mixture was purified by flash column chromatography (EtOAc/hexanes: 1/1) to give [2] as a light yellow oil (2.5 g, 88%). ¹H NMR (400 MHz, CDCl₃) δ 6.76 (br, 1H, NH); 3.26 (t, 2H, J=7.3 Hz); 2.67-2.59 (m, 4H); 2.46 (t, 2H, J=7.3 Hz); 2.22-2.16 (m, 4H); 1.42 (s, 9H); ¹³C NMR (77 MHz, CDCl₃) δ 207.1; 155.9; 79.6; 55.3 (2C); 53.4; 41.2 (2C); 38.9; 28.4 (3C). ESI-TOF MS calculated for C₁₂H₂₂N₂O₃, m/z 242.32, found 243.43 [MH]⁺.

tert-butyl (2-(4-(phenylamino)piperidin-1-yl)ethyl)carbamate [3]. Aniline (750 mL, 8.2 mmol) was taken up in methylene chloride in a 100 mL round-bottom flask equipped with a stir bar. The light brown solution was placed on an ice bath and treated dropwise with acetic acid (AcOH, 460 mL, 8.2 mmol). To the mixture, tert-butyl (2-(4-oxopiperidin-1-yl)ethyl)carbamate [2] (2 g, 8.2 mmol) was added as a solution in methylene chloride, followed by the careful, slow addition of sodium cyanoborohydride (NaBH₃CN, 773 mg, 12.3 mmol) in small portions. The reaction mixture was stirred at 80° C. for 14 hours. After this time, methanol was added to the mixture, and all contents transferred to a separatory funnel. The mixture was partitioned (CH₂Cl₂//saturated NaHCO₃). Once neutralized, the organic phase was washed with brine, dried over Na₂SO₄ and concentrated in vacuo to give a light brown oil. The oily mixture was purified by flash column chromatography (EtOAc/hexanes: 7/3) to give [3] as a light yellow oil (2.25 g, 86%). ¹H NMR (400 MHz, CDCl₃) δ 7.37 (d, 2H, J=7.8 Hz); 7.27-7.25 (m, 3H); 6.35 (br, 1H, NH); 5.21 (br, 1H, NH); 3.84 (m, 1H); 3.77-3.58 (m, 4H); 3.21-3.14 (m, 4H); 2.31-2.26 (m, 4H); 1.43 (s, 9H); ¹³C NMR (77 MHz, CDCl₃) δ 156.7; 147.6; 129.5 (2C); 120.8; 113.5 (2C); 79.5; 64.3; 53.9; 52.0 (2C); 39.7; 30.3 (2C); 26.8 (3C). ESI-TOF MS calculated for C₁₈H₂₉N₃O₂, m/z 319.23, found 320.17 [MH]⁺.

tert-butyl (2-(4-(N-phenylpropionamido)piperidin-1-yl)ethyl)carbamate [4]. Compound [3] (1.5 g, 4.7 mmol) was dissolved in methylene chloride in a 100 mL round bottom flask equipped with a small stir bar and was treated with diisopropylethylamine (DIPEA, 1.64 mL 9.4 mmol). The solution was cooled with an ice bath and treated dropwise with propionyl chloride (0.81 mL, 9.4 mmol). The resulting mixture was stirred for 2 h at ambient temperature. The mixture was transferred to a separatory funnel and partitioned (CH₂Cl₂//H₂O). The organic phase was washed with brine, saturated NaHCO₃, dried over anhydrous Na₂SO₄ and evaporated in vacuo to give a yellow oil that was purified by flash column chromatography (EtOAc/hexanes: 4/6) to furnish [4] as a light yellow oil (1.68 g, 95%). ¹H NMR (400 MHz, CDCl₃) δ 7.40-7.27 (m, 3H); 7.26 (d, 2H, J=7.8 Hz); 4.72 (m, 1H); 3.98 (t, 2H, J=7.1 Hz); 3.27-3.17 (m, 4H); 2.82-2.74 (m, 4H); 2.15-2.08 (m, 4H); 1.40 (s, 9H); 1.01 (t, 3H, J=7.1 Hz); ¹³C NMR (77 MHz, CDCl₃) δ 174.6; 155.9; 137.9; 128.3 (2C); 128.0; 127.5 (2C); 81.5; 62.5; 53.9; 53.4 (2C); 40.1; 28.2; 26.8 (3C); 25.9 (2C); 10.3. ESI-TOF MS calculated for C₂₁H₃₃N₃O₃, m/z 319.23, found 320.17 [MH]⁺.

N-(1-(2-aminoethyl)piperidin-4-yl)-N-phenylpropionamide [5]. Trifluoroacetic acid (TFA, 20% volume) was added to a solution of compound [4] (1 g, 3.1 mmol) in dichloromethane (40 ml). The resultant solution was stirred at room temperature. Upon complete disappearance of starting material, the solvent was removed under vacuum. The crude reaction mixture was co-evaporated with large volumes of dichloromethane several times to yield a white solid that was placed under vacuum overnight. The resulting white solid (1.45 g, quantitative) was used without further purification. ¹H NMR (400 MHz, CDCl₃) δ 12.2 (br, 1H); 7.37-7.29 (m, 3H); 7.03 (d, 2H, J=7.8 Hz); 4.62 (m, 1H); 3.83 (t, 2H, J=7.1 Hz); 3.18-3.05 (m, 4H); 2.75-2.62 (m, 4H); 2.15-2.08 (m, 4H); 0.98 (t, 3H, J=7.1 Hz); ¹³C NMR (77 MHz, CDCl₃) δ 177.3; 161.4; 159.3; 136.2; 126.2 (2C); 126.1; 125.9 (2C); 113.2; 112.9; 85.7; 62.3; 54.2; 53.4 (2C); 41.2; 27.2; 23.9 (2C); 10.3. ESI-TOF MS calculated for C₂₀H₂₇F₆N₃O₅, m/z 503.19, found 377.25 [MH-TFA]⁺, 276.33 [MH-2×TFA]⁺. 4-oxo-5-((2-(4-(N-phenylpropionamido)piperidin-1-yl)ethyl)amino)pentanoic acid [6]. To a solution of amine [5] (1 g, 2 mmol) dissolved in dichloromethane with pyridine (0.64 mL, 6 mmol), glutaric anhydride (228 mg, 2 mmol) was added dropwise. The mixture was stirred at room temperature overnight. After disappearance of starting material, the solvent was concentrated to dryness; the residue was co-evaporated with toluene (3×). The mixture was transferred to a separatory funnel and partitioned (CH₂Cl₂//H₂O). The organic phase was washed with brine, saturated NaHCO₃, dried over anhydrous Na₂SO₄ and evaporated in vacuo to give [6] as a white solid (780 mg, quantitative). Compound [6] was used without further purification. ¹H NMR (400 MHz, CDCl₃) δ 7.35-7.31 (m, 3H); 7.02 (d, 2H, J=7.8 Hz); 4.79 (m, 1H); 4.02 (t, 2H, J=7.1 Hz); 4.57-4.52 (m, 2H); 2.91-2.85 (m, 2H); 2.62-2.51 (m, 4H); 2.25-2.13 (m, 2H); 1.99-1.75 (m, 8H); 0.98 (t, 3H, J=7.1 Hz); ¹³C NMR (77 MHz, CDCl₃) δ 178.4; 174.6; 172.6; 137.9; 128.9 (2C); 128.1; 127.5 (2C); 83.5; 59.1; 52.0 (2C); 39.2; 35.6; 32.3; 28.2; 27.6 (2C); 20.3; 10.2. ESI-TOF MS calculated for C₂₁H₃₁N₃O₄, m/z 389.23, found 390.21 [MH]⁺.

tert-butyl (5-oxo-5-((2-(4-(N-phenylpropionamido)piperidin-1-yl)ethyl)amino)pentanoyl) glycylglycylglycylglycinate [7]. The carboxylic acid [6] (500 mg, 1.28 mmol) was dissolved in dichloromethane, followed by the subsequent addition of 2-(1H-benzotriazol-1-yl)-1,1,3,3-tetramethyluronium hexafluorophosphate (HBTU, 729 mg, 1.92 mmol) and the appropriate amine (Gly)₄-OtBu (Pravetoni et al., The Journal of Pharmacology and Experimental Therapeutics 2012; 341:225-232) (387 mg, 1.28 mmol). N,N-Diisopropylethylamine (DIPEA, 403 mL, 2.31 mmol) was then added to the mixture. The solution was stirred overnight at room temperature. The mixture was transferred to a separatory funnel and partitioned (CH₂Cl₂//H₂O). The organic phase was washed with brine, saturated NaHCO₃, dried over anhydrous Na₂SO₄ and evaporated in vacuo. The resulting mixture was purified by SiO₂ flash column chromatography (EtOAc/hexanes: 6/4) to provide [7] as a light yellow oil (637 mg, 74%). ¹H NMR (400 MHz, CDCl₃) δ 7.41-7.39 (m, 3H); 7.08 (d, 2H, J=7.9 Hz); 6.13 (br, 1H, NH); 4.62 (m, 1H); 3.71-3.65 (m, 10H); 3.30-3.26 (m, 2H); 3.00-2.98 (m, 2H); 2.55-2.49 (m, 2H); 2.32-2.15 (m, 6H); 1.93-1.80 (m, 6H); 1.43 (s, 9H); 0.98 (t, 3H, J=7.1 Hz); ¹³C NMR (77 MHz, CDCl₃) δ 177.9; 174.8; 172.5; 171.0 (3C); 169.5; 137.8; 128.4 (2C); 128.2; 127.6 (2C); 83.3; 81.8; 59.1; 52.1 (2C); 42.4; 42.7; 41.0; 39.6; 35.5; 34.6; 28.7 (3C); 27.5 (2C); 22.2; 10.1. ESI-TOF MS calculated for C₃₃H₅₁N₇O₈, m/z 673.38, found 674.47 [MH]⁺.

(5-oxo-5-((2-(4-(N-phenylpropionamido)piperidin-1-yl)ethyl)amino)pentanoyl) glycylglycylglycylglycine [8]. TFA (1 ml) was added to a solution of compound [7] (300 mg, 0.47 mmol) in dichloromethane (4 ml). The mixture was stirred at room temperature overnight. Upon complete disappearance of starting material, the solvent was removed under vacuum. The residue was re-dissolved in dichloromethane and evaporated again. This operation was repeated twice to afford a pale-yellow paste that was purified by SiO₂ flash column chromatography (MeOH/CH₂Cl₂: 2/98) to give [8] as a white solid (266 mg, 92%). ¹H NMR (400 MHz, CDCl₃) δ 7.39-7.36 (m, 3H); 7.02 (d, 2H, J=7.9 Hz); 4.59 (m, 1H); 3.69-3.62 (m, 10H); 3.21-3.18 (m, 2H); 2.98-2.92 (m, 2H); 2.47-2.42 (m, 2H); 2.29-2.16 (m, 6H); 1.91-1.82 (m, 6H); 0.97 (t, 3H, J=7.1 Hz); ¹³C NMR (77 MHz, CDCl₃) δ 178.1; 174.6; 174.2; 172.6; 169.2 (3C); 137.9; 128.6 (2C); 128.4; 127.5 (2C); 83.4; 59.8; 53.3 (2C); 42.7; 42.9; 41.3; 39.7; 35.3; 34.7; 27.6 (2C); 22.5; 10.3. ESI-TOF MS calculated for C₂₉H₄₃N₇O₈, m/z 617.32, Found 618.31 [MH]⁺. Analytical calculation for C₂₉H₄₃N₇O₈: C, 56.39; H, 7.02; N, 15.87. found: C, 56.34; H, 6.99; N, 15.91.

Vaccines. The F hapten (FIG. 1B) was conjugated through carbodiimide (EDAC; Sigma-Aldrich, St. Louis, Mo.) chemistry as previously described for other opioid-based haptens (Pravetoni et al., Vaccine 2012; 30:4617-4624; Pravetoni et al., The Journal of Pharmacology and Experimental Therapeutics 2012; 341:225-232). Briefly, 5 mM of hapten was reacted with a 52 mM concentration of EDAC in 0.1 M IVIES ((4-morpholineethanesulfonic acid) buffer at pH 4.5, and stirred for 5 minutes at room temperature. Bovine serum albumin (BSA), ovalbumin (OVA), KLH, or sKLH were then added to the reaction mixture at amounts of 2.8, 1.9, 2.8, and 2.8 mg, respectively, in a final volume of 1 ml, and stirred for 3 hours at room temperature as described (Pravetoni et al., The Journal of Pharmacology and Experimental Therapeutics 2012; 341:225-232). Bioconjugation efficacy was indirectly measured by assessing the haptenation ratio of F-BSA by comparing the molecular weight of the unconjugated and conjugated BSA by MALDI-TOF. Conditions optimized for F-BSA led to a haptenation ratio of 8. Haptenation ratios were not determined for KLH because its molecular weight is too large to be measured by MALDI-TOF.

Drugs. Fentanyl, oxycodone, and heroin were obtained through the National Institute on Drug Abuse Drug Supply Program (Bethesda, Md.) or Sigma-Aldrich (St. Louis, Mo.). Drug doses and concentrations are expressed as the weight of the base.

Brain and serum fentanyl concentration. Fentanyl concentrations were measured by gas chromatography mass spectrometry (GC/MS) using a modified procedure (Huynh et al., J Pharm Biomed Anal 2005; 37:1095-1100). Briefly, trunk blood was collected and centrifuged following experimentation at 3100×g for 3 minutes at 4° C. Internal standard (D5-fentanyl, 50 mL of 1 mg/mL) was added to all serum and standard samples. Then, 0.15 mL 1.0M NaOH and 3.5 mL n-heptane with 3% 2-butanol was added to 0.5 mL serum samples. Samples were capped and rotated at approximately 15 RPM for 30 minutes on orbital shaker (Orbitron Rotator II, Model260250; Boekel Scientific, Feasterville, Pa.) and then centrifuged for 5 minutes at 1500 g. The lower aqueous phase was frozen using a mixture of dry ice and acetone for 10 minutes. The solvent layer was transferred and placed on an N-Evap at 45° C. until the solvent was completely evaporated. The solvent was reconstituted in 50 ml of ethyl acetate, briefly vortexed, and then centrifuged for 5 minutes at 1500 g. Samples were injected into the gas chromatograph/mass spectrometer for analysis.

Antibody characterization. Antibody characterization included determination of antibody titers, estimated minimum antibody concentrations, and stoichiometry.

Antibody titers. Fentanyl-specific serum IgG antibody titers were measured as previously described for other hapten conjugates (Raleigh et al., The Journal of Pharmacology and Experimental Therapeutics 2013; 344:397-406; Raleigh et al., PloS One 2017; 12:e0184876). Briefly, F conjugated to OVA (F-OVA) was used as the coating antigen for mouse studies and conjugated to BSA (F-BSA) for rat studies. Coating antigen was diluted in 0.05 M carbonite buffer pH 9.6, coated onto 96 well ELISA plates (Jackson ImmunoResearch Laboratories, Inc., West Grove, Pa.), and stored overnight at 4° C. Plates were blocked with 1% gelatin in phosphate buffered saline with Tween 20 (PBST) for 1 hour and then stored overnight at 4° C. The next day various dilutions of sera in 0.05 M PBST were added to the wells and plates incubated for 2 hours at room temperature. After washing the plates, Fc-specific goat anti-rat or anti-mouse IgG coupled to horseradish peroxidase (Jackson ImmunoResearch Laboratories, Inc., West Grove, Pa.), were added and incubated at overnight at 4° C. O-phenylenediamine (OPD) was used (SIGMAFAST™ tablet set, Sigma Life Sciences, St Louis, Mo.) as the reaction substrate. After 30 minutes of incubation, 2% oxalic acid was added to stop the enzymatic reaction. Plates were read at 492 nm on a BioTek PowerWave XS (BioTek Instruments Inc., Winooski, Vt.).

Estimated minimum antibody concentrations. The minimal fentanyl-specific antibody concentrations present in serum were estimated by assuming that the difference in serum fentanyl concentration in vaccinated and control rats represents the fentanyl retained in serum by the binding capacity contributed by antibody. This value was calculated by subtracting the mean control vaccine group serum fentanyl concentration from the individual serum fentanyl concentration from each vaccinated rat and multiplying by the molecular weight of IgG (150,000 Da) divided by two binding sites per IgG.

The total number of moles per kilogram of opioid-specific IgG in rats vaccinated with F-KLH or F-sKLH in Experiments 2 and 4 was calculated as the product of the estimated antibody concentration in serum and the reported IgG volume of distribution (131 mL/kg) in rats (Bazin-Redureau et al., The Journal of Pharmacy and Pharmacology 1997; 49:277-281; Pravetoni et al., The Journal of Pharmacology and Experimental Therapeutics 2012; 341:225-232).

Experiment 1: Effect of F-KLH on opioid distribution and antinociception after single fentanyl challenge in mice. Male BALB/c mice (Envigo, Madison, Wis.) 5 to 6 weeks old were housed in groups of 4 under 12/12-hour standard light/dark cycle. Groups of 8 mice were immunized s.c. with F-KLH or control KLH vaccine containing 25 μg immunogen and 0.5 mg aluminum hydroxide (Alhydrogel, Invitrogen, San Diego, Calif.). Vaccine was administered s.c. on day 0, 14, and 28. On day 35, blood was collected via submandibular bleeding for antibody characterization. On day 42 baseline antinociception was assessed by placing mice on a 54° C. hotplate and measuring the latency to respond, defined as the time until a response of hindpaw lick or jumping (Pravetoni et al., The Journal of Pharmacology and Experimental Therapeutics 2012; 341:225-232). Testing was terminated at 60 seconds to avoid tissue damage. Mice received a 0.05 mg/kg s.c. dose of fentanyl. This dose was chosen because it elicited submaximal latency to respond on the hotplate in a pilot study. 30 minutes after dosing, mice were placed on the hotplate again to measure latency to respond. The percentage maximum possible effect (% MPE) was calculated as the post-test latency minus the pre-test latency divided by the maximum time (60 seconds) minus the pre-test latency times 100. Immediately after hotplate testing mice were anesthetized with isoflurane and blood and brain collected to assess drug levels.

Experiment 2: Efficacy and selectivity of F-KLH on opioid distribution and antinociception in rats challenged with fentanyl, heroin, and oxycodone. Male Holtzman rats (Envigo, Madison, Wis.) weighing 200-225 g were double housed with 12/12-hour standard light/dark cycle. Groups of 12 rats were immunized s.c. with F-KLH or control KLH vaccine containing 25 μg immunogen and 0.5 mg aluminum hydroxide. Vaccine was administered i.m. on day 0, 21, and 42. On day 49, blood was collected via tail vein for antibody characterization. Hotplate testing was identical to that in Experiment 1, except that rats received 0.035 mg/kg s.c. dose of fentanyl. This dose was chosen because it elicited submaximal latency to respond on the hotplate in a pilot study. On day 51, KLH-immunized and F-KLH immunized rats were split into two groups and half (n=6 for KLH and n=6 for F-KLH treated rats) received 1 mg/kg heroin s.c. and the other half (n=6 for KLH and n=6 for F-KLH treated rats) received 2.25 mg/kg oxycodone s.c. and tested on the hotplate as in Experiment 1. These doses were chosen because they produce significant antinociception and have previously been use to test heroin and oxycodone vaccine efficacy in rats (Pravetoni et al., The Journal of Pharmacology and Experimental Therapeutics 2012; 341:225-232).; Pravetoni et al., J Med Chem 2013; 56:915-923; Raleigh et al., The Journal of Pharmacology and Experimental Therapeutics 2013; 344:397-406). On day 58, rats were given a 0.05 mg/kg i.v. infusion of fentanyl for 1 minute and blood and brain were collected 4 minutes later. The i.v. route was chosen, in contrast to the s.c. route commonly used for antinociception, because it provides the most rigorous challenge with regard to route, and because it corresponds to how it is most commonly encountered as an adulterant. The dose was chosen because it was a large fentanyl dose capable of testing vaccine efficacy and because fentanyl levels in serum and brain in rats at this i.v. dose was already well described (Hug and Murphy, Anesthesiology 1981; 55:369-375).

Experiment 3: Fentanyl-induced antinociception and respiratory depression after repeated fentanyl challenges in rats. Sprague Dawley rats were used in Experiments 3 and 4 because Holtzman rats became temporarily unavailable. Male Sprague Dawley rats (Envigo, Madison, Wis.) weighing 200-225 g were double housed with 12/12-hour standard light/dark cycle. To determine a fentanyl dose range that produced a wide range of antinociception and respiratory depression, 8 rats were tested on the hotplate and oximeter following successive fentanyl s.c. doses. Baseline antinociception (prior to fentanyl administration) was assessed. Immediately following the hotplate test rats were placed in a 12 inch×12 inch enclosed chamber to prevent and a MouseOx (STARR Life Sciences Corp., Oakmont, Pa.) arterial oxygen saturation (SaO₂) monitor was placed via neck collar for at least 1 minute to ensure stable readings were obtained and baseline SaO₂ was measured as described (Raleigh et al., PloS One 2017; 12:e0184876). SaO₂, breath rate, and heart rates were recorded as the mean of the last 10 seconds, which correspond to 10 measurements. Rats then received fentanyl every 17 minutes s.c. so that their cumulative fentanyl dose at successive intervals was 12.5, 25, 50, and 100 mg/kg. Fifteen minutes after each fentanyl dose, antinociception and SaO2 were again measured; this accounted for 2 minutes, resulting in the 17 minutes fentanyl dosing interval. Heart rate was obtained from the oximeter. Rats received 0.1 mg/kg naloxone s.c. immediately after the final antinociception and SaO₂ measures were obtained. The naloxone dose was chosen based on previous data (Raleigh et al., PloS One 2017; 12:e0184876) and is comparable to the maximum recommended dose (Wong (2012) Naloxone and nalmefene, in Poisoning and Drug Overdose (K. R. O ed) pp≤514-517, McGraw Hill, New York). Antinociception and cutaneous SaO₂ were again measured 15 minutes after naloxone administration. Rats were awake throughout hotplate and oximeter testing.

Experiment 4: Efficacy of F-sKLH on opioid distribution, antinociception, and respiratory depressive effects after repeated fentanyl challenges in rats. Male Sprague Dawley rats (Envigo, Madison, Wis.) weighing 200-225 g were double housed with 12/12-hour standard light/dark cycle. Groups of 8 rats were immunized i.m. with F-sKLH or control sKLH vaccine containing 25 μg immunogen and 0.5 mg aluminum hydroxide on days 0, 21, 42, and 63. On day 70, blood was collected via tail vein for antibody characterization. On day 77, rats were tested on the hotplate and oximeter using the same protocol as in Experiment 3. Blood and brain were collected at the end of the experiment to measure fentanyl concentrations in these tissues.

Statistical analysis. Fentanyl-specific serum antibody titers, opioid levels, and hotplate antinociception for Experiments 1 and 2 were compared between groups using unpaired t tests with Welsh's correction. For Experiment 3, latency to respond in the hotplate nociception test and SaO₂ were compared using a repeated measures one-way ANOVA using Dunnett's multiple comparison test. For Experiment 4, latency to respond in the hotplate nociception test and SaO₂ were compared between groups over time by 2-way ANOVA using Sidak's multiple comparisons test while within group comparisons were done using Dunnett's multiple comparison test. Effective dose of fentanyl that caused 50% maximal effect (ED₅₀) on the antinociceptive hotplate test was performed using nonlinear regression analysis using the model [Agonist] vs. response—Variable slope (four parameters) with the ceiling parameter set as a constant equal to 60 seconds (maximal latency to respond). ED₅₀ for % SaO₂ and breath rate could not be measured because minimum and maximum values could not be established. All statistics were performed using Prism (version 8.0a.91; GraphPad, San Diego, Calif.).

### Results

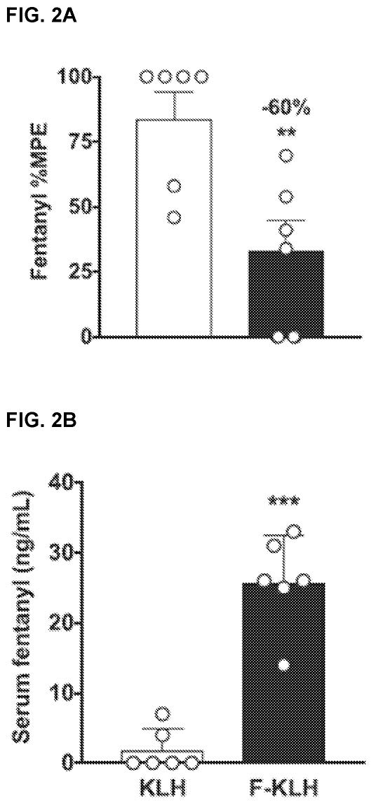

Experiment 1: Efficacy of F-KLH on opioid distribution and antinociception after single s.c. injection of fentanyl in mice. Mice vaccinated with F-KLH had 92±62×10³ (mean±SD) fentanyl-specific antibody titers. Estimated minimum antibody concentrations were 5.3±1.5 mg/mL. Because of the manner in which this minimum concentration was estimated (from the concentration of fentanyl retained in serum by antibody), actual antibody concentration may have been higher. F-KLH vaccination significantly reduced fentanyl-induced antinociceptive effects on the hotplate by 60% compared to controls (FIG. 2A, p<0.01). Serum fentanyl concentrations were significantly increased in F-KLH vaccinated mice compared to controls (FIG. 2B, p<0.001).

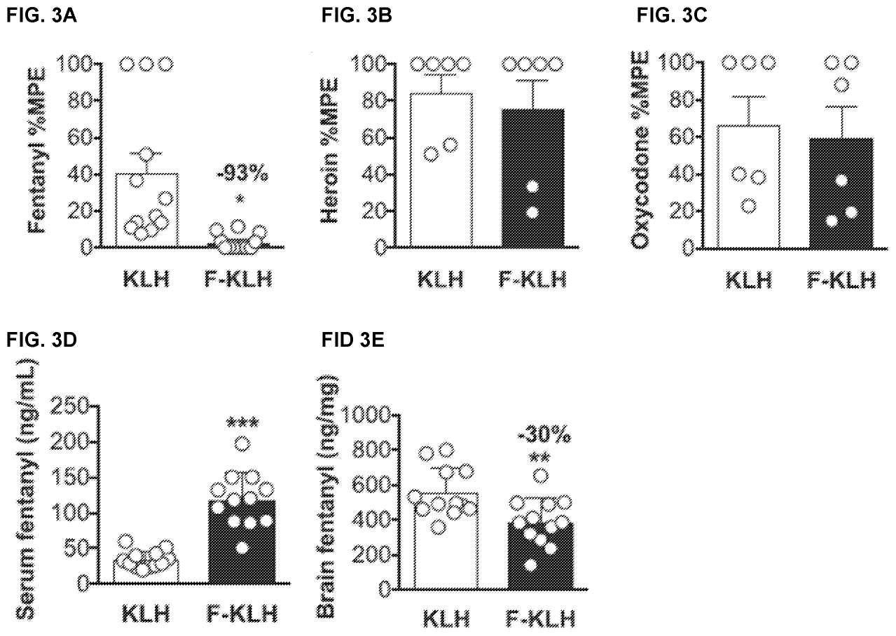

Experiment 2: Efficacy of F-KLH on opioid distribution and antinociception after single s.c. injection of fentanyl in rats. Rats vaccinated with F-KLH had 9.0±4.4×10³ fentanyl-specific antibody titers. Estimated minimum antibody concentrations were 18.6±8.5 mg/mL. The molar ratio of the fentanyl dose (0.035 mg/kg) to the estimated antibody binding sites in F-KLH vaccinated rats was 3.2. F-KLH vaccination significantly reduced fentanyl-induced antinociceptive effects on the hotplate by 93% compared to controls (FIG. 3A, p<0.05). F-KLH vaccination had no effect on heroin- or oxycodone-induced antinociception (FIG. 3B and FIG. 3C, p=0.64 and 0.76, respectively). One week after antinociceptive testing, rats received a 1-minute infusion of 0.05 mg/kg i.v. fentanyl. The molar ratio of the fentanyl dose (0.05 mg/kg) to the estimated antibody binding sites in F-KLH vaccinated rats was 4.6. Serum fentanyl concentrations were significantly increased (FIG. 3D, p<0.001) and brain fentanyl concentrations were decreased by 30% (FIG. 3E, p<0.05) compared to controls.

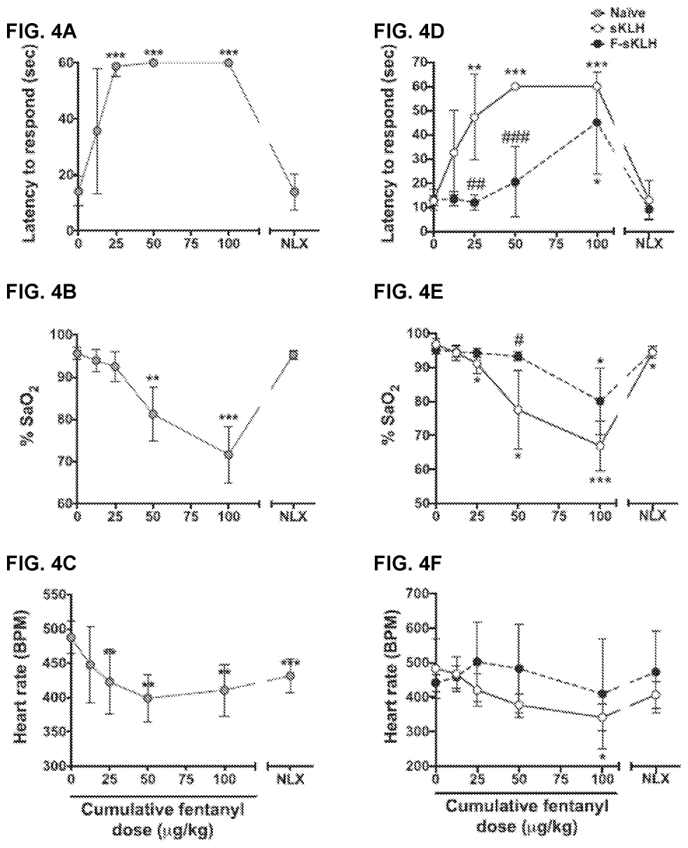

Experiment 3: Fentanyl-induced antinociception and respiratory depression after cumulative s.c. fentanyl dosing in rats. Latency to respond on the hotplate was significantly increased following the 25, 50, and 100 mg/kg cumulative doses (FIG. 4A, p<0.001 at all three doses) compared to baseline latencies. Naloxone returned latencies to respond on the hotplate back to baseline levels. Percent SaO2 levels were significantly reduced following the 50 and 100 mg/kg cumulative doses (FIG. 4B, p<0.01 and p<0.001, respectively). Naloxone reversed % SaO2 levels to baseline values. Heart rate (in beats per minute) was significantly lowered following the 25, 50, and 100 μg/kg cumulative doses (FIG. 4C, p<0.01 at all three doses). Naloxone treatment did not reverse BPM back to baseline (p<0.001).

Experiment 4: Efficacy of F-sKLH on opioid distribution, antinociception, and respiratory depressive effects after cumulative s.c. fentanyl dosing in rats. Rats vaccinated with F-sKLH had 25±9.6×10³ fentanyl-specific antibody titers. Estimated minimum antibody concentrations were 68.2±46 mg/mL. The molar ratio of the fentanyl dose (0.1 mg/kg) to the estimated antibody binding sites in F-sKLH vaccinated rats was 2.5. F-sKLH attenuated fentanyl-induced antinociception by shifting the latency to respond dose-response curve to the right on the hotplate after increasing cumulative doses of fentanyl (FIG. 4D, vaccination, F(1,14)=42.0, p<0.001; interaction, F(5,70)=10.4, p<0.001; fentanyl dose, F(3.35,46.9)=40.9, p<0.001). Fentanyl significantly increased latency to respond following 25 mg/kg in sKLH vaccinated rats, but only after 100 mg/kg in the F-sKLH group, compared to their baseline values. These values represent an ED₅₀ of 0.02±0.01 mg/kg in the sKLH group and 0.08±0.06 mg/kg in the F-sKLH group, decreasing fentanyl potency by a 5.4-fold shift in the presence of fentanyl-specific antibodies. Naloxone completely reversed fentanyl-induced antinociception in both groups. F-sKLH significantly reduced fentanyl-induced respiratory depression following the 50 mg/kg fentanyl dose and shifted the % SaO2 dose-response curve rightward (FIG. 4E, vaccination, F(1,14)=17.7, p<0.001; interaction, F(5,70)=9.1, p<0.001; fentanyl dose, F(2,28.1)=49.7, p<0.001). Fentanyl significantly decreased % SaO2 following 25 mg/kg in sKLH vaccinated rats, but only after 100 mg/kg in the F-sKLH group, compared to their baseline values. Naloxone completely reversed fentanyl-induced % SaO2 in the F-sKLH vaccinated group, but not in controls (p<0.05). There was no effect of F-sKLH on fentanyl-induced bradycardia (FIG. 4F, vaccination, F(1,14)=2.09, p=0.17; interaction, F(5,70)=3.25, p<0.05; fentanyl dose, F(1.65,23.0)=4.5, p<0.05). However, fentanyl significantly decreased the heart rate in sKLH, but not in F-sKLH, treated rats compared to their baseline (p<0.05). Naloxone completely reversed fentanyl-induced bradycardia in both groups.

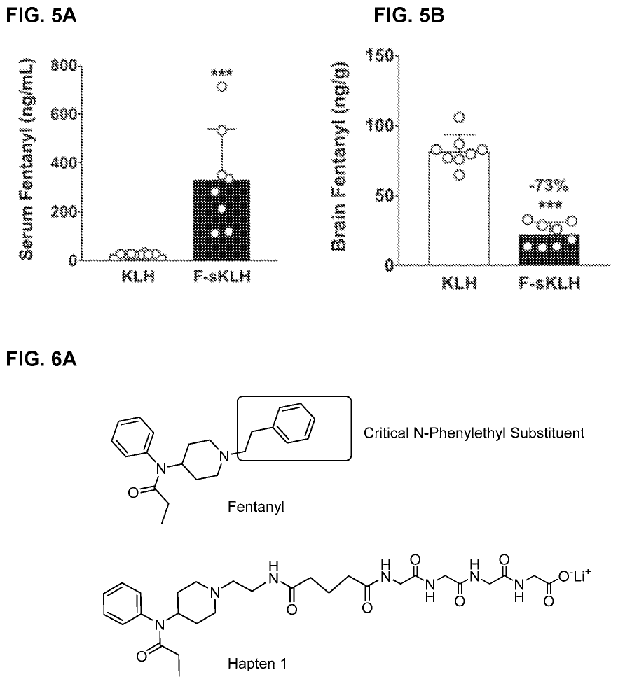

Serum fentanyl concentrations were significantly higher in F-sKLH vaccinated rats compared to controls (p<0.001) following the 100 mg/kg cumulative s.c. fentanyl dose (FIG. 5A). Brain fentanyl concentrations were 73% lower in F-sKLH vaccinated rats compared to controls following the 100 mg/kg cumulative s.c. fentanyl dose (FIG. 5B, p<0.01).

### Discussion

This study Example describes that: 1) F-sKLH attenuated fentanyl-induced respiratory depression and antinociception in rats; 2) F-KLH selectively reduced the antinociceptive effects of fentanyl, but not of equianalgesic doses of heroin or oxycodone in vivo; 3) F-sKLH preserved naloxone's reversal of fentanyl-induced effects; and 4) F-KLH and F-sKLH reduced brain fentanyl levels following administration of large fentanyl doses. These data demonstrate that a fentanyl vaccine can effectively reduce fentanyl's effects, including its respiratory depressive effects, when formulated either on the native KLH or its GMP-grade version sKLH, which were both formulated in FDA-approved alum adjuvant and delivered i.m., the most common route of immunization for vaccines.

Fentanyl-induced overdose is characterized by marked respiratory depression, leading to death if respiration is not restored either through reversal (naloxone) or ventilation and oxygenation (Boyer, NEJM 2012; 367:146-155). Fentanyl-induced respiratory depression may occur at different doses in humans than in rats. In this Example, fentanyl induced respiratory depression at s.c. cumulative fentanyl doses above 12.5-25 mg/kg in sKLH treated and naïve rats. In humans, sublingual doses of 800 mg (11 mg/kg in a 70 kg human) caused respiratory depression after 2 hours in all 12 subjects (Lister et al., J Clin Pharmacol 2011; 51:1195-1204). In another study, apnea was reported in human subjects at an i.v. fentanyl dose as low as 2.9 mg/kg, and that prolonged apnea occurred at 7.1 mg/kg, leading the investigators to halt using this dose for the remainder of the study (Dahan et al., British Journal of Anaesthesia 2005; 94:825-834). In this same study, respiratory depression measured in rats was reported in the range of 50 to 90 mg/kg i.v., and although these doses were infused over a period of 20 minutes to avoid death, suggesting a 10 times higher potency to induce respiratory depression in humans compared to rats.

In this Example, F-sKLH attenuated fentanyl-induced respiratory depression up to a cumulative s.c. fentanyl dose of 50 mg/kg (4 times higher than in sKLH group), suggesting that fentanyl vaccines can block fentanyl-induced respiratory depression at doses considerably larger than respiratory-depressive doses in human. Another fentanyl vaccine has been shown to reduce the respiratory depressive effects of fentanyl in dogs following an i.v. dose of 5 mg/kg fentanyl (Torten et al., Nature 1975; 253:565-566). However, the dogs that this study were passively immunized and the antibody concentration was not specified. It has not yet been established how effective F-sKLH would be in limiting respiratory depression following i.v. fentanyl doses. The difference seen in vaccine efficacy between these two studies may be due to differences in fentanyl pharmacokinetics in these species, vaccine formulations, or route of fentanyl administration. Nevertheless, these data suggest that fentanyl vaccines can block fentanyl's respiratory-depressive effects following various routes of administration.

Hotplate antinociception, a surrogate for addiction-related behaviors because it is mediated in the central nervous system by opioid receptors (Le Bars et al., Pharmacological Reviews 2001; 53:597-652), was also reduced by vaccination with F-KLH and F-sKLH. F-KLH reduced fentanyl-induced antinociception by 60% in mice given 0.05 mg/kg fentanyl s.c. and 90% in rats given 0.035 mg/kg fentanyl s.c. In Experiment 4, the protective effects of F-sKLH on the hotplate antinociceptive assay extended up until a 100 mg/kg fentanyl s.c. dose (a 5.4-fold shift compared to KLH controls). Similar findings have been reported in two previous studies. In one study, a fentanyl vaccine was able to prevent fentanyl's antinociceptive effect 90 seconds following an i.v. fentanyl dose of 100 mg/kg in mice (Torten et al., Nature 1975; 253:565-566). In another study, a fentanyl vaccine was able to shift the hotplate dose-response curve ED₅₀ by 24-fold following cumulative s.c. doses of up to 1 mg/kg fentanyl in mice (Bremer et al., Angew Chem Int Ed Engl 2016; 55:3772-3775).

F-KLH blocked the analgesic activity of fentanyl, but not equianalgesic doses of heroin or oxycodone. The antinociceptive potencies (ED₅₀) of fentanyl, heroin, and oxycodone (0.06, 0.62, and 1.53 mg/kg, respectively) are significantly different (Peckham and Traynor, The Journal of Pharmacology and Experimental Therapeutics 2006; 316:1195-1201.) and much larger heroin and oxycodone doses are required to achieve antinociception equivalent to that of fentanyl. In vitro selectivity of antibodies elicited by the fentanyl vaccine was not measured in the current study, but due to the high selectivity of antibodies towards their targeted compound and hapten for other similarly designed opioid vaccines, cross-reactivity towards heroin and oxycodone is unexpected. These data suggest that a fentanyl vaccine would likely block clinically relevant doses of fentanyl, but not heroin or oxycodone.

Naloxone is important for reversing fentanyl-induced respiratory depression and overdose. Part of establishing the usefulness of opioid vaccines in humans is ensuring that the effects of naloxone are maintained because naloxone may need to be administered more than once due to fentanyl's high potency. To this end, it is noteworthy that vaccination with F-KLH in the current study did not interfere with naloxone efficacy for reversing fentanyl respiratory depression.

Both F-KLH and F-sKLH reduced fentanyl distribution to brain following a large i.v. fentanyl dose and a large cumulative s.c. fentanyl dose, respectively, despite molar ratio excesses of fentanyl dose to estimated antibody binding sites by at least 2.5. The i.v. dose given to rats in the current study was approximately 7 times higher than the i.v. dose that causes severe respiratory depression in humans (Dahan et al., British Journal of Anaesthesia 2005; 94:825-834). Reduction of brain fentanyl has been reported by another group using one fentanyl vaccine following a 0.2 mg/kg s.c. fentanyl dose (Bremer et al., Angew Chem Int Ed Engl 2016; 55:3772-3775), but this effect was not replicated in another study by the same group using a differentfentanyl vaccine following a 0.1 mg/kg i.v. fentanyl dose (Hwang et al., ACS Chem Neurosci 2018; 9:1269-1275). While there was no clear explanation for that discrepancy, differences in fentanyl vaccine potency between those two vaccines and differences in dosing route are most likely the cause. Drug dosing route has been shown to affect addiction vaccine efficacy in at least one oxycodone vaccine which showed greater reduction of brain oxyocodone concentrations following s.c. dosing (44% reduction) than following i.v. dosing (12% reduction), likely due to slower absorption following s.c. dosing compared to i.v. dosing (Raleigh et al., The Journal of Pharmacology and Experimental Therapeutics 2018; 365:346-353). This route effect could have contributed to the large (90%) decrease in brain fentanyl levels with vaccination in the current study when fentanyl was administered s.c.

The doses of fentanyl used in the current study generated clinically-relevant serum fentanyl concentrations in the range of 5 to 30 ng/mL. In humans, therapeutic serum concentrations of fentanyl show a Cₘₐₓ ranging from 0.2 to 0.9 ng/mL following sublingual, intranasal, transmucosal fentanyl, or i.v. administration of fentanyl (Nave et al., Drug Deliv 2013; 20:216-223 Parikh et al., Clin Ther 2013; 35:236-243), while unintentional overdose of transdermal patches show fentanyl blood concentrations ranging from 5 to 28 ng/mL (Jumbelic, Am J Forensic Med Pathol 2010; 31:18-21) and respiratory depression at plasma concentrations of 2.5 to 6.3 ng/mL in humans (Mildh et al., Anesthesia and Analgesia 2001; 93:939-946.).

One limitation is that control rats in Experiment 2 had an unexpectedly low % MPE of 41%. This low % MPE may be due to fentanyl's small therapeutic window and small deviations in the fentanyl dose given to rats may have greatly affected hotplate antinociception results in the current study. Nevertheless, F-KLH significantly reduced fentanyl-induced antinociception.

In summary, the data of this Example suggest that F-sKLH could be used as a prophylactic to reduce the side effects of fentanyl, alone or in conjunction with naloxone, and as a potential therapeutic for fentanyl abuse in humans.

### Example 2—A Re-Formulated Fentanyl Vaccine Alters Fentanyl Distribution and Protects Against Fentanyl-Induced Effects in Mice and Rats

This Example describes the development of a novel vaccine formulation including a re-formulated F₁ hapten conjugated to the GMP-grade subunit keyhole limpet hemocyanin (sKLH) or to CRM from various sources via an optimized conjugation strategy.

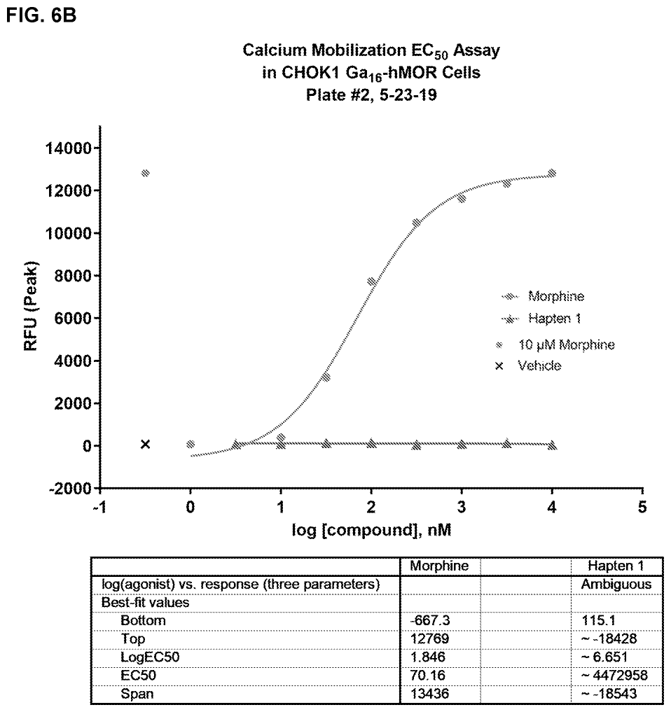

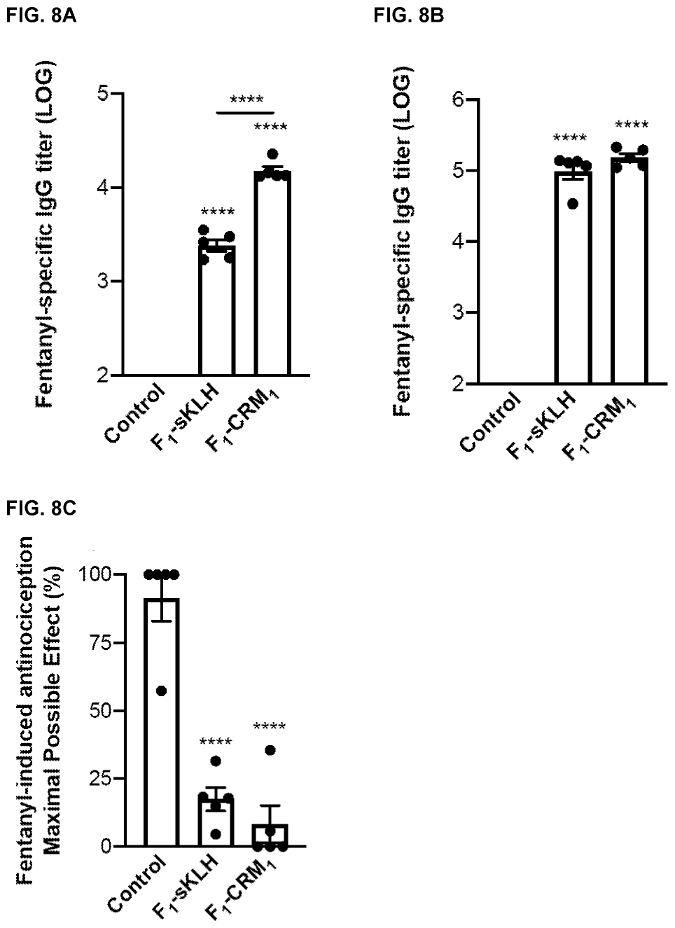

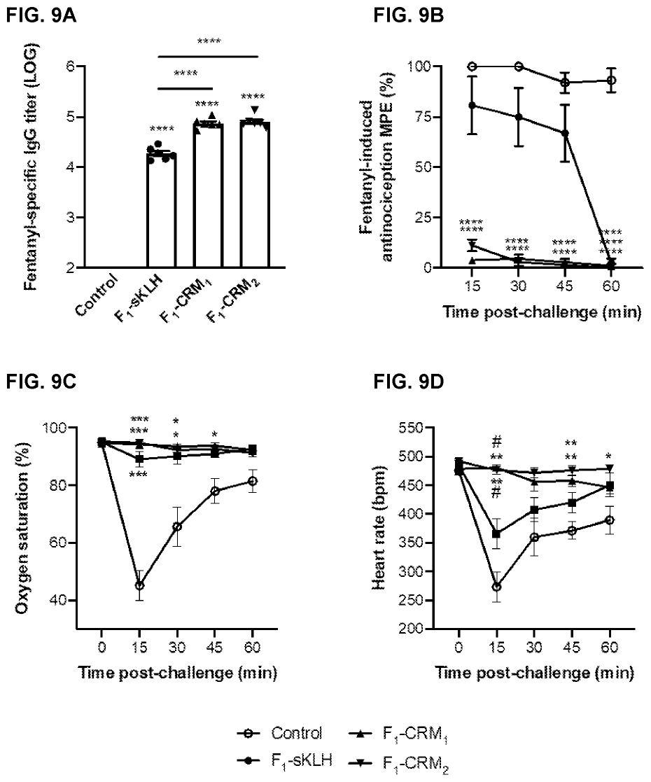

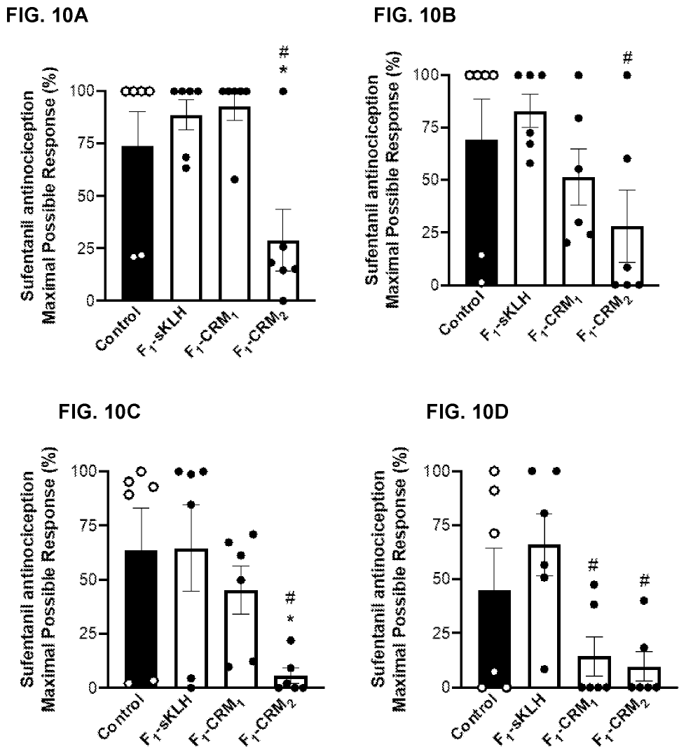

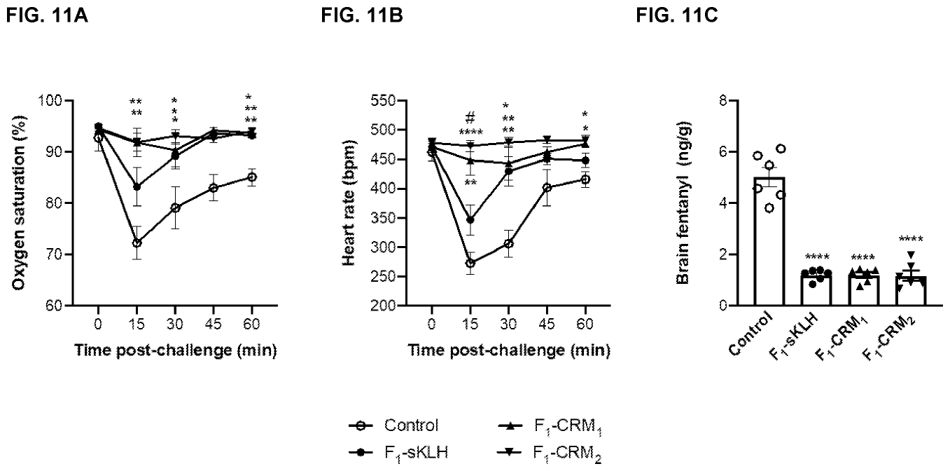

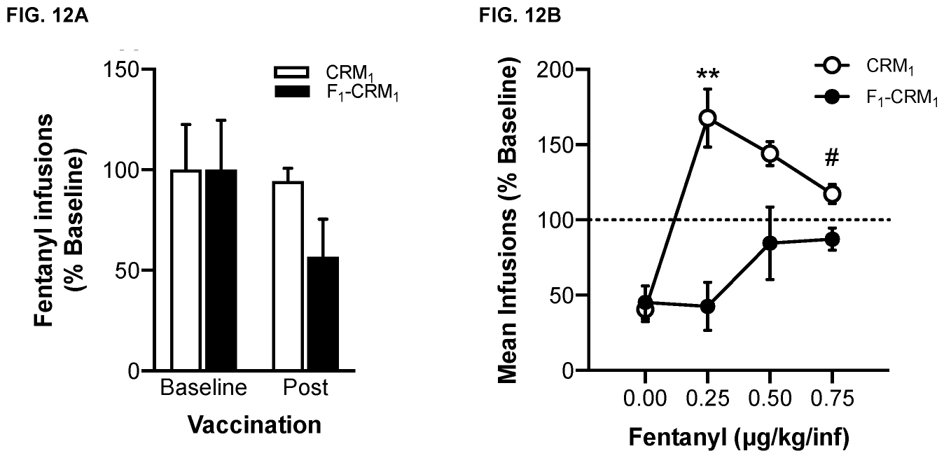

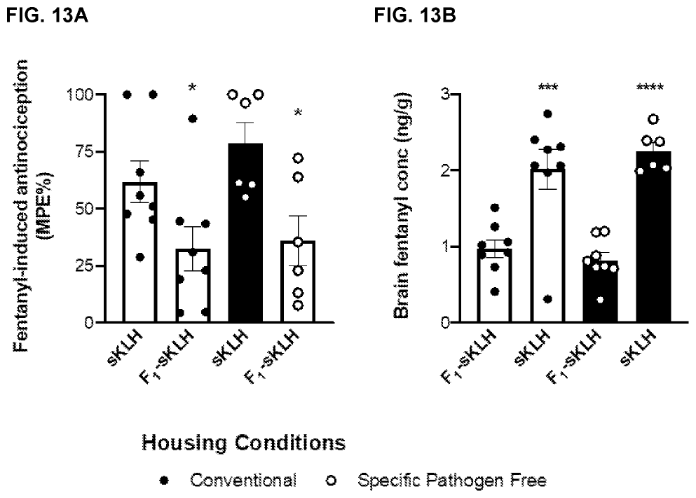

In this Example, F₁-sKLH, F₁-CRM₁, and F₁-CRM₂ conjugates were characterized for their biophysical properties and then tested in mice and rats (FIG. 6-FIG. 14). Immunized mice and rats were challenged for fentanyl-induced behavior and toxicity commonly associated with opioid use disorders and overdose. Immunization with F₁-CRM₁, and F₁-CRM₂ showed increased efficacy over the previously characterized F₁-sKLH (see Example 1) in blocking fentanyl-induced antinociception (FIG. 8, FIG. 9, FIG. 11), respiratory depression, and bradycardia (FIG. 9, FIG. 11). Vaccination was also effective in reducing sufentanil-induced antinociception (FIG. 10), supporting the use of fentanyl-based vaccines against other potent fentanyl analogs. Vaccination was effective in rats with ongoing fentanyl intravenous self-administration (FSA), as shown by the F₁-CRM₁ ability to reduce FSA (FIG. 12). Furthermore, rats immunized with F₁-CRM₁, discontinued FSA under a dose-reduction protocol supporting the notion that immunization against fentanyl does not cause an increase in FSA to compensate for dose reduction and overcome vaccine efficacy (FIG. 12). Vaccination against fentanyl was equally effective in mice housed in conventional or specific pathogen conditions (FIG. 13) suggesting that the environment or microbiota may not affect vaccine efficacy against fentanyl and other opioids. Moreover, these results suggest that an immunocompromised patient (for example, an HIV⁺ patient) may still be benefit from immunization against fentanyl or other opioids. Finally, efficacy of F₁-CRM₁, and to a lesser extent F₁-sKLH, was enhanced by an immunomodulator of the interleukin 4 (IL-4) confirming that IL-4 is either a pharmacological target for enhancing vaccine efficacy against opioids and that IL-4 is a biomarker of vaccine efficacy against fentanyl and other opioids (FIG. 14).

### Materials and Methods

Drugs and Reagents. Fentanyl and sufentanil were obtained from the University of Minnesota Pharmacy. Drug doses and concentrations are expressed as weight of the free base. Subunit Keyhole Limpet Hemocyanin (sKLH) was purchased as GMP-grade source (Biosyn, Carlsbad, Calif.), cross-reactive material (CRM) from diphtheria toxin was purchased either as E. coli-expressed CRM (EcoCRM) (Fina Biosolutions, Rockville, Md.) or CRM₁₉₇ (PFEnex, San Diego, Calif.). CRM from these different sources is described as CRM₁ and CRM₂, respectively.

Animals. Adult male BALB/c mice (Jackson Laboratories, Bar Harbor, Me.) and adult male Sprague-Dawley rats (Envigo, Huntingdon, United Kingdom) were housed in standard 12/12 hours light/dark cycle and fed ad libitum. Mice were 6 weeks old on arrival, and rats were 2 months old on arrival. Animals were immunized immediately after 1 week of habituation. Mice and rats were housed under conventional housing, unless specified that specific pathogen free (SPF) environment was required by the experimental conditions (results related to housing effects are shown in FIG. 13).

Hapten Synthesis (F₁). Fentanyl-based hapten F₁ was synthesized as described in Example 5 and as shown in FIG. 1D (Scheme 2).

F₁ hapten activity at the Mu Opioid Receptor (MOR). To test whether F₁ hapten had any functional activity at the MOR, the F₁ hapten was tested in vitro in a calcium mobilization assay involving Chinese Hamster Ovary (CHO) cells co-expressing the human MOR and Gα16, a promiscuous G protein, as previously described (Raleigh et al. PLoS One. 2017; 12(12):e0184876). Results are shown in FIG. 6.

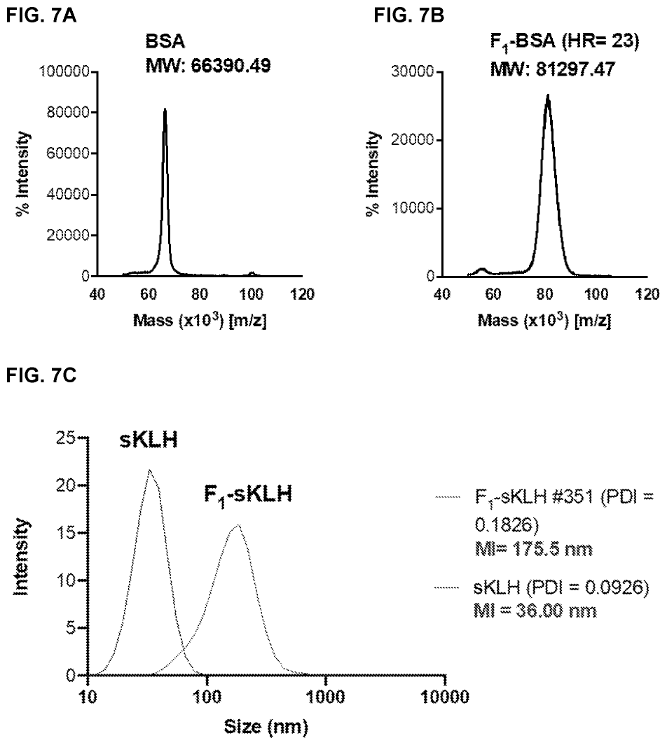

Conjugation of the F₁ hapten. Conjugation was performed according to the protocol previously described for either OXY(Gly)₄OH, M(Gly)₄OH, or F(Gly)₄OH haptens (Baruffaldi et al. Mol Pharm. 2018; 15(11):4947-4962; Raleigh et al. J Pharmacol Exp Ther. 2019; 368(2):282-291; Baruffaldi et al. Mol Pharm. 2019; 16(6):2364-2375) with modifications to improve haptenization ratio and yield. Briefly, the F₁ hapten was dissolved at a concentration of 5.2 mM in 0.1 M IVIES buffer pH 4.5 containing 10% DMSO and was activated by carbodiimide coupling chemistry using N-ethyl-N′-(3 dimethylaminopropyl) carbodiimide hydrochloride (EDAC, Sigma-Aldrich, St. Louis, Mo.) cross-linker at a final concentration of 208 mM. The mixture was left reacting for 10 minutes at room temperature (RT). BSA, sKLH, or CRM were added at a final concentration of 2.8 mg/ml and the reactions were stirred for the following 3 hours at RT. The final conjugates were ultrafiltered using Amicon filters with 50 kDa or 100 kDa molecular cutoff depending on the carrier protein dimensions: after having replaced IVIES buffer with phosphate-buffered saline (PBS) 0.1 M pH 7.2, the resulting solutions were stored at +4° C. To provide a clear suspension of conjugate for the CRM-containing conjugates, 250 mM sucrose was included in both reacting and storage buffer as a stabilizing agent. Conjugates containing either BSA or CRM were characterized by MALDI-TOF to determine their molecular weight (MW) and haptenization ratio. Because of their larger MW, the sKLH and F₁-sKLH were characterized for size by dynamic light scattering (DLS) as described previously (Baruffaldi et al. Mol Pharm. 2018; 15(11):4947-4962). Results are shown in FIG. 7 and Table 2.

Immunization. Mice were immunized intramuscularly (i.m.) with 60 μg of F₁ hapten-conjugate or unconjugated sKLH, CRM₁ or CRM₂ as a control. Conjugates were adsorbed with 30 μg of alum (Alhydrogel® 85, Brenntag, Essen, Germany) and PBS to a final volume of 60 and delivered 30 μL per leg. Mice were immunized on days 0, 14, and 28. In a specific experiment, the F₁-sKLH and the F₁-CRM₁ were co-administered with a mouse monoclonal antibody (mAb) anti-interleukin 4 (anti-IL-4 or αIL-4). The anti-IL-4 mAb was administered before and after the first vaccination as described previously (Laudenbach et al. Sci Rep. 2018; 8(1):5508). Results related to the effect of anti-IL-4 mAb are shown in FIG. 14.

Rats were immunized i.m. with 60 μg of F₁ hapten-conjugate or unconjugated sKLH, CRM₁ or CRM₂ as a control. Conjugates were adsorbed with 90 μg of alum and PBS to a final volume of 150 μL and delivered to one leg. Rats were immunized on days 0, 21, 42 and 63 or 0, 21, 42, 63, and 84 as detailed in specific experiments.

Antibody Analysis. Serum IgG antibody analysis was performed via indirect ELISA after blood collection using facial vein sampling in mice or tail vein sampling in rats. 96-well plates were coated with 5 ng/well of F₁-BSA conjugate or unconjugated BSA as a control. Conjugates were diluted in 50 mM Na₂CO₃, pH 9.6 (C3041-100CAP, Sigma Aldrich, St. Louis, Mo.) and blocked with 1% porcine gelatin. Serum was incubated on the plate and then washed and incubated with an HRP-conjugated goat anti-mouse IgG (115-035-008, Jackson ImmunoResearch Laboratories, West Grove, Pa.) or goat anti-rat IgG (112-005-008 Jackson ImmunoResearch Laboratories, West Grove, Pa.) to assess hapten-specific serum IgG antibody levels using statistical analysis as described in Pravetoni et al., J. Med. Chem. 2013; 56(3):915-23. Serum hapten-specific IgG₁ and IgG₂ₐ titers were obtained as described in Laudenbach et. al., Sci. Rep. 2018; 8(1):5508. Competitive binding ELISA was used to determine IC₅₀ for fentanyl or its analogs as previously described (Raleigh et al. J Pharmacol Exp Ther. 2019; 368(2):282-291). Results are shown in FIG. 8A, FIG. 8B, FIG. 9A, FIG. 14A, FIG. 14B, and Table 2.

Effect of vaccine on opioid-induced analgesia via hot plate. To determine the efficacy of the vaccine to block opioid-induced analgesia, a hot plate test was performed. Rodents were allowed to acclimate to the testing environment for 1 hour prior to measuring baseline. Rodents were placed on a hot plate (Columbus Instruments, Columbus, Ohio) set to 54° C. and removed after displaying a lift or flick of the hindpaw. Mice were vaccinated on days 0, 14 and 28. On day 35, baselines were measured and mice were then given 0.05 mg/kg fentanyl, delivered subcutaneously. Hotplate responses were measured 30 minutes after injection. Rats were vaccinated on days 0, 21, 42 and 63. Starting at day 49, animals were tested repeatedly once a week. Drugs were given as a single subcutaneous dose. The initial drug tested was fentanyl at a dose of 0.075 mg/kg (week 1), followed by 0.008 mg/kg sufentanil (week 2), 0.5 mg/kg alfentanil and a final challenge of 0.1 mg/kg fentanyl (week 3). The latency to respond on the hotplate was measured at 15, 30, 45, and 60 minutes post-drug administration. Data are displayed as mean percentage error (MPE) calculated as: (postdrug latency−baseline latency)/(maximal cutoff−baseline latency)×100.

Results are shown in FIG. 8C, FIG. 9B, FIG. 10, FIG. 13A

Effect of vaccine to protect against opioid-induced respiratory depression and bradycardia. To assess the efficacy of immunization with F₁-containing conjugates against opioid-induced respiratory depression and bradycardia, oxygen saturation and heart rate (BPM) were measured by oximetry before and after drug administration. Oximetry was measured using a MouseOx Plus (Starr Life Sciences, Oakmont, Pa.). Rodents were allowed to acclimate to the testing environment for 1 hour prior to measuring baseline. After baseline, rats were given a single bolus dose of drug delivered subcutaneously. The initial drug tested was fentanyl at a dose of 0.075 mg/kg, followed by 0.008 mg/kg of sufentanil, 0.5 mg/kg of alfentanil and a final fentanyl challenge of 0.1 mg/kg. Oximetry measurements were taken at 15, 30, 45, and 60 minutes post-drug administration. Results are shown in FIG. 9C, FIG. 9D, FIG. 11A, FIG. 11B.

Vaccine protection against fentanyl intravenous self-administration (FSA). To assess the efficacy of immunization with F₁-containing conjugates individuals with ongoing fentanyl use disorders, immunized rats were challenged in the fentanyl self-administration (FSA). First rats were implanted with jugular catheters. Rats were then trained to self-administer fentanyl (1 μg/kg/infusion) under a fixed-ratio (FR) 3 schedule during daily 120-minute sessions. Once FSA stabilized, rats were immunized IM with either CRM₁ or F₁-CRM₁ (n=6/group) on days 0, 21, 42, 63, and 84 and their FSA activity recorded. After rats received the 5^{th} injection (day 84), rats were started on a dose reduction protocol where fentanyl dose/infusion was progressively reduced (by 0-1 μg/kg/infusion) during FSA following a weekly dose reduction (that is, the fentanyl dose was reduced every Monday of every week). Results are shown in FIG. 12.

Statistical analysis. Fentanyl-specific serum antibody titers (LOG), fentanyl serum or brain concentrations, latency to respond in the hotplate nociception test, oxygen saturation (SaO2), and heart rate (beats per minute, BPM) on single time points were compared using a one-way ANOVA paired with Dunnett's multiple comparison test, whereas comparison over multiple time points were analyzed by two-way ANOVA paired with Tukey's multiple comparison test. All statistics were performed using Prism (version 8.0a.91; GraphPad, San Diego, Calif.).

### Discussion

The main results from the studies detailed in this Example were the following: 1) although F₁ hapten does not activate the Mu Opioid Receptor (MOR) (FIG. 6), it is effective in generating polyclonal antibodies that bind fentanyl, sufentanil, and potentially other fentanyl-like analogs, and is effective at reducing their toxicity in vivo. Lack of activity at MOR will increase safety of the F₁ hapten during manufacturing; 2) the synthesis of the F₁ hapten can be further improved (FIG. 1D) to facilitate synthesis scale-up and to yield a purer compound that facilitates conjugation to sKLH and CRM avoiding precipitation or aggregates (Table 1 and FIG. 7); 3) the F₁ hapten can be further improved to facilitate conjugation to a variety of carrier proteins, including sKLH, and by improving its haptenization ratio (Table 1 and FIG. 7); 4) re-formulation of the lead F₁-sKLH conjugate vaccine by conjugation of the F₁ hapten to CRM yielded a conjugate that can be characterized by MALDI-TOF rather than DLS; 5) the re-formulated F₁-CRM vaccine induced higher fentanyl-specific serum IgG antibody titers than F₁-sKLH in mice and rats, and the resulting antibodies were more effective in reducing fentanyl-induced antinociception in mice and rats, 6) F₁-CRM conjugates were more effective than F₁-sKLH in reducing fentanyl-induced antinociception, respiratory depression, and bradycardia in rats (FIG. 9 and FIG. 11); 7) F₁-CRM conjugates were more effective than F₁-sKLH sufentanil-induced antinociception in rats, suggesting that anti-fentanyl vaccines can be used to counteract toxicity from other fentanyl analogs (FIG. 10); 8) F₁-CRM conjugates were more effective than F₁-sKLH or F₁-nKLH in stimulating polyclonal antibody responses with higher affinity for fentanyl, and its analogs, and lower affinity for off-target opioids (Table 2); 9) the efficacy of F₁-CRM may depend upon the source of CRM, further supporting that the optimal formulation of a hapten:carrier:adjuvant ratio is paramount to increased efficacy; 10) vaccination with F₁-CRM decreased fentanyl intake in rats actively engaging in FSA and prevented compensation compared to control rats (FIG. 12); 11) the fentanyl vaccine was equally effective in mice housed under different conditions that may affect the microbiome, suggesting that microbial environment of the host may not negatively impact vaccine efficacy (FIG. 13); 12) the efficacy of F₁-CRM, but not necessarily F₁-sKLH, was enhanced by an immunomodulator of IL-4 (FIG. 14), further supporting the necessity of optimizing vaccine formulations by tweaking each individual component (for example, hapten:carrier:adjuvant:immunomodulator). These pre-clinical data further support use of biomarkers predictive of vaccine efficacy against fentanyl and other opioids. The immunomodulatory effects of IL-4 on vaccine efficacy against fentanyl suggest that IL-4, and other potential biomarkers as detailed in Table 3, may be used to identify subjects amenable to vaccination against fentanyl and other opioids. Overall these data support the translation of anti-fentanyl vaccines and further inform their use in the clinic.

TABLE 1
Characterization of conjugates containing the F₁ hapten.

| | | Estimated | |
|---|---|---|---|
| | | Haptenation | Precipitate/ |
| Conjugate | MW (MALDI-TOF) | Ratio (HR) | Aggregate |
| F₁-BSA | 81320.2656 | 22 | No |
| F₁-sKLH | N/A | N/A | No |
| F₁-CRM₁ | 70049.7734 | 18 | No |
| F₁-CRM₂ | 66578.7500 | 12 | No |

F₁-hapten was conjugated to subunit KLH (sKLH), and either CRM₁ (E. coli expressed CRM from FinaBio) or CRM₂ (Pfenex).

TABLE 2
Affinity of antibodies for fentanyl, analogs and off-target opioids. Sera from A) mice
and B and C) rats immunized with conjugates containing fentanyl hapten Fi conjugated to either
nKLH, sKLH, CRM₁, or CRM₂ Analysis was performed by either competitive binding ELISA or
biolayer interferometry (BLI) to determine either IC₅₀ or K_{d} for fentanyl or its analogs.
A. Target opioids: mouse

| Vaccine | | | |
|---|---|---|---|
| Formulation | ID | Coating | Fentanyl IC₅₀ (nM) |
| F₁-sKLH/alum/IM | 6501, 7699 | F-BSA | 204.4, 61.8 |
| F₁-sKLH/alum/IM | 6501, 7699 | F-BSA | 155.3, 598.6 |
| F₁-sKLH/alum/IM | 6501, 7699 | F-BSA | 11.8, 5.3 |

B. Target opioids: rats

| Vaccine | | Fentanyl IC₅₀ | Fentanyl | Sufentanil | Alfentanil | Remifentanil |
|---|---|---|---|---|---|---|
| Formulation | Coating | (μM) | IC₅₀ (nM) | IC₅₀ (μM) | IC₅₀ (μM) | IC₅₀ (μM) |
| *F₁-sKLH | F₁-Bsa | 0.0142 ± | 14.2 ± 4.14, | 17.78 ± 0.59 | 60.23 ± 39.77 | 64.72 ± 35.29 |
| | | 0.00414, | 50.3 ± 41.2 | | | |
| | | 0.0503 ± 10.0412 | | | | |
| *F₁-CRM₁ | F₁-Bsa | 0.5303 | 530 ± 167 | 14.60 ± 6.86 | >100 | >100 |
| | | 0.530 ± 1.67 | | | | |
| *F₁-CRM₂ | F₁-Bsa | 0.1132 | 113.2 ± 63.4 | 65.13 ± 34.88 | 66.55 ± 33.46 | >100 |
| | | 0.113 ± 0.0634 | | | | |
| **F₁-nKLH | F₁-Bsa | 25.0 | | N/A | N/A | N/A |
| **F₁-sKLH | F₁-Bsa | 995.6 | | 1785.0 | 4734.0 | 1747.0 |

*These formulations contain the F1 hapten generated as described in Example 5 and Scheme 2. See also Baehr et al.,
JPET2020 (published online Sep. 26, 2020).
**These formulaitions contain historical data obtained with the original F₁ hapten, and data reported in Raleigh et al., JPET
2018; 365:346-353. These latter data were generated from pooled rat sera. Polyclonal sera from rats immunized with the
series of conjugates containing F1 haptens were analyzed by competitive binding ELISA, and when possible compared to
a monoclonal antibody (mAb) isolated from mice immunized with Fi-sKLH. The anti-F mAb shows Kd of 1.59-0.53 nM for
fentanyl as measured by biolayer interferometry (BLI).
C. Off-target opioids: rats

| Vaccine | | Buprenorphine | Naloxone | Naltrexone | Methadone |
|---|---|---|---|---|---|
| Formulation | Coating | IC₅₀ (mM) | IC₅₀ (mM) | IC₅₀ (mM) | IC₅₀ (mM) |
| *F₁-sKLH | F₁-Bsa | >0.3^{#} | >10 | >10 | >10 |
| **F₁-nKLH | F₁-Bsa | 1.21 | 2.71 | 1.54 | 1.0 |
| **F₁-sKLH | F₁-Bsa | N/A | 0.81 | 3.31 | N/A |

*These formulations contain the newly synthesized F hapten.
**These formulations contain historical data obtained with the original F₁ hapten. These latter data were generated from pooled rat sera.
^{#}The maximal concentration of buprenorphine tested was 0.3 mM because of the commercially available drug stock concentration.
The maximal concentrations of naloxone, naltrexone, and methadone were 10 mM.

TABLE 3
Biomarkers predictive of vaccine efficacy
against fentanyl and other opioids

| Biomarker | Antibodies | Efficacy |
|---|---|---|
| Opioid- and hapten-specific B cells^{a} | ↑ | ↑ |
| Sex (female)^{b} | ↑ | ↑ |
| Environment & Microbiome | No Effect | ? |
| IL-4 modulation^{c} | ↑ | ↑ |
| IL-2 modulation^{c} | No Effect | No Effect |
| PD-L1 modulation^{c} | ↓ | ↓ |
| IL-6 modulation | No Effect | No Effect |
| ICOS^{c} | ↓ | ↓ |
| Fc gamma (γ) receptor I-IV | No Effect | ↑ |
| Fcγ neonatal receptor | ↓ | ↓ |

^{a}Laudenbach et. al. J. Immunol. 2015; 94(12): 5926-36; Laudenbach et al. Vaccine 2015; 33(46): 6332-39; Laudenbach et. al., Sci. Rep. 2018; 8(1): 5508; and Taylor JJ et. al. J. Immunol. Methods 2014; 405: 74-86.
^{b}Crouse et. al. NPJ Vaccines 2020; 5: 99.
^{c}Laudenbach et. al., Sci. Rep. 2018; 8(1): 5508.

### Example 3

This Example describes further characterization of the re-formulated F₁ hapten (Example 3A-Example 3A) and the re-formulated F₁ hapten conjugated to a carrier (Example 3C). The re-formulated F₁ hapten and the conjugates were prepared as described in Example 2 and Example 5.

### Example 3A—Thermogravimetric Analysis (TGA) of F₁ Hapten

TGA Q500 V20.13 Build 39 (TA Analysis, New Castle, Del.) instrument was used to determine the percentage weight loss (%) of the fentanyl hapten F₁ (lyophilized powder) during heating. 1.999 mg of F₁ were heated from 15° C. to 300° C. with a heating rate of 10° C./min and a nitrogen gas purge of 60 mL/min. FIG. 15A shows that F₁ lithium salt contained 4-5% water (w/w) and started decomposing at ˜240° C.

### Example 3B—Differential Scanning Calorimetry (DSC) of F₁ Hapten

DSC Q1000 V9.9 Build 303 (TA Analysis, New Castle, Del.) instrument was used to record the DSC thermograms. 12.700 mg of fentanyl hapten F₁ were accurately weighed and sealed in an aluminum hermetic pan, poked on the top to allow for water escape during heating. The sample was heated and cooled three times from 0° C. to 150-200° C. using a heating rate of 20° C./minute and a nitrogen gas purge of 25 mL/minute. The onset temperatures of the thermal events were extrapolated with the TA Universal Analysis software. Results are shown in FIG. 15B. Cycle 1 shows a great endothermic event linked to water desorption. The glass transition temperature (Tg) of the dehydrated F1 sample is shown in Cycle 2 at approximately 120° C. The endothermic peak in Cycle 3 (onset ˜180° C.) is F₁ melting peak.

### Example 3C—MALDI-TOF and DSC Analysis of F₁-Carrier Conjugate

MALDI-TOF was performed as described in Baruffaldi et al. Mol. Pharmaceutics 2018; 15(11):4947-4962. DSC was performed as described in Example 3B.

Results are shown in FIG. 15C-FIG. 15F.

These data indicate that F₁ conjugated to different carriers may have a different physical chemical profile. For example, F₁ behaves differently when attached to sKLH, or different CRM versions, as assessed by MALDI-TOF and DLS for MW and size or aggregation state.

In addition, these data indicate that F₁-sKLH is not stable overtime because its peak shifted in size from T=0 to T=1 month. In contrast, F₁-CRM₁, and F₁-CRM₂ were stable for at least 1 month.

Further, as shown by the size of F₁-CRM₁, and F₁-CRM₂ (as assessed by either DLS or MALDI-TOF), these conjugates can be sterile filtered by 0.45 nm or 0.22 nm pore-size filters. In contrast, F₁-sKLH cannot be analyzed by MALDI-TOF nor sterile filtered because of its high molecular weight and aggregation status.

### Example 4

This Example describes further testing of the vaccine formulation including re-formulated F₁ hapten conjugated to CRM, prepared as described in Example 2 and Example 5.

### Example 4A—Pre-Existing Immunity Against Carrier Proteins do not Interfere with Vaccination Against Fentanyl

Because it is likely that human subjects have been previously exposed to common carrier proteins through standard pediatric or occupational immunization (for example, diphtheria or tetanus prophylaxis), whether pre-exposure to carrier proteins would interfere with vaccination was tested. A similar study previously found that pre-existing immunity to carrier proteins may affect the efficacy of anti-nicotine vaccines (McCluskie et al., Immunopharmacol Immunotoxicol. 2016; 38(3):184-96).

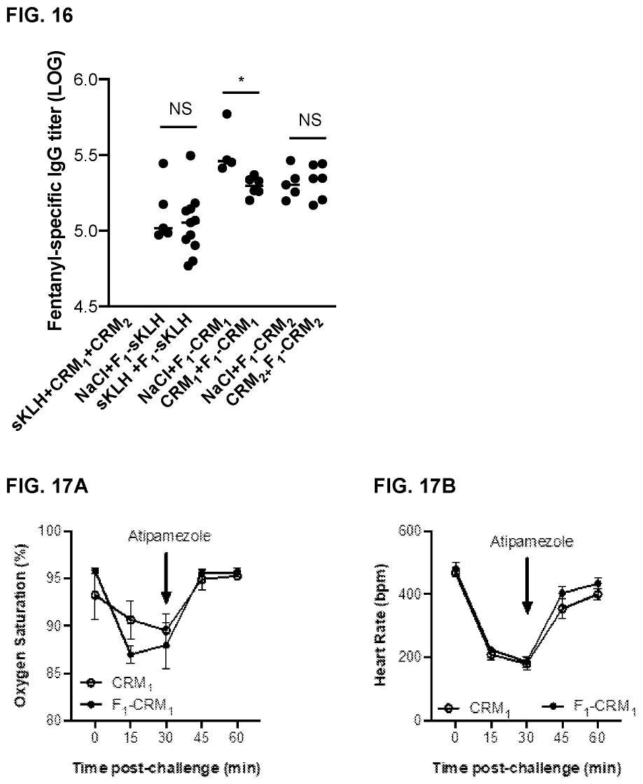

Mice were first immunized i.m. on day −14 with either saline control or 60 μs of carrier protein, then immunized using the corresponding F₁-carrier protein conjugate on days 0, 14 and 28. Pre-exposure to either sKLH or CRM₂ had no effect on fentanyl-specific serum IgG antibody titers, while pre-exposure to CRM₁ negatively impacted development of fentanyl-specific antibody titers (FIG. 16).

These data indicate that both sKLH and CRM₂ are viable carrier proteins for future vaccine development as pre-exposure in animals did not significantly diminish vaccine efficacy. In addition, these date show 1) vaccination with F₁-CRM did not interfere with pharmacological activity of commonly used anesthetics (FIG. 17); and 2) vaccination with F₁-CRM did not interfere with pharmacological activity of opioid agonists, and antagonists used in treatment of opioid use disorders, overdose reversal, or pain management (FIG. 18).

### Example 4B—Vaccination Against Fentanyl does not Interfere with Anesthesia

To provide proof of safety and to guide clinical use of anti-fentanyl vaccines, it is critical to identify lead vaccines that do not interfere with medications for OUD, standard critical care, and pain management. To determine whether anti-fentanyl vaccines would interfere with anesthesia, rats were immunized with either CRM₁ or F₁-CRM₁ and then challenged weekly with a series of anesthetics. Anesthetic efficacy was measured by respiratory depression and bradycardia. Vaccination with F₁-CRM₁ did not interfere with anesthesia induced by dexmedetomidine (0.25 mg/kg, FIG. 17A-FIG. 17C), and did not prevent its reversal by the standard dexmedetomidine-reversal agent atipamezole (1 mg/kg). Accordingly, no differences between control and active vaccine groups were found when rats were anesthetized with ketamine (75 mg/kg, FIG. 17D-FIG. 17E), propofol (100 mg/kg, FIG. 17F), or isoflurane (FIG. 17G-FIG. 17H). Following multiple challenges with anesthetics, the F₁-CRM vaccinated rats retained efficacy against fentanyl (0.05 mg/kg, FIG. 17I-FIG. 17K). These data demonstrate that F₁-CRM₁ is selective for fentanyl and does not interfere with commonly used anesthetics.

### Methods:

Vaccine efficacy against antinociception induced by target and off-target opioids in mice and rats. The effect of candidate vaccines against antinociception induced by either target or off-target opioids was evaluated in the hot plate test of centrally-mediated analgesia. Mice and rats were allowed to acclimate to the testing environment for 1 hour prior to measuring baseline. Rodents were placed on a hot plate (Columbus Instruments, Columbus, Ohio) set to 54° C. and removed after displaying a lift or flick of the hindpaw, or reaching the maximal cutoff of 60 seconds in mice or 30 seconds in rats to avoid thermal tissue damage. In mice, testing was initiated after the third immunization (day 35). After measurement of baseline latencies, mice were challenged with fentanyl (0.05-0.1 mg/kg, s.c.) and their hotplate responses recorded at 30 minutes post-drug challenge. In rats, testing was initiated after the 3^{rd} immunization (day 49). To determine the efficacy of candidate vaccines against selected target opioids, rats were tested repeatedly once a week with drug challenges including fentanyl (0.075-1.0 mg/kg, s.c.), sufentanil (0.008 mg/kg, s.c.), and alfentanil (0.5 mg/kg, s.c.) as detailed in each experiment, and the latency to respond was measured at 15, 30, 45, and 60 minutes post-drug administration. At completion of the behavioral assessment (mice=31 minutes, and rats=61 minutes, which includes the 60 second maximal cutoff threshold), trunk blood and brain were collected for assessment of fentanyl concentrations. To test whether vaccination interfered with selected opioid agonists, and the reversal of their effects by naloxone, rats immunized with either CRM₁ or F₁-CRM₁ were challenged weekly with oxycodone (2.25 mg/kg, s.c.), heroin (0.9 mg/kg, s.c.), methadone (2.25 mg/kg, s.c.), and fentanyl (0.1 mg/kg, s.c., positive control) and hotplate responses were recorded at 30 minutes post-drug challenge. After receiving oxycodone or heroin, rats were given naloxone (0.1 mg/kg, s.c.) and their responses recorded for an additional 15 minutes. In both mouse and rat studies, data are displayed as maximal possible effect (MPE %) calculated as: (post-drug latency−baseline latency)/(maximal cutoff-baseline latency)×100.

Vaccine efficacy against respiratory depression and bradycardia induced by target opioids and off-target anesthetics in rats. To assess the effect of candidate vaccines against respiratory depression and bradycardia induced by either target opioids or off-target anesthetics, pulse oximetry was used to measure oxygen saturation (SaO2%), breath rate (breaths per minute, brpm), and heart rate (beat per minute, bmp) before and after drug challenges. Oximetry was measured using a MouseOx Plus pulse oximeter (Starr Life Sciences, Oakmont, Pa.). Rats were allowed to acclimate to the testing environment for 1 hour prior to measuring baseline. After baseline recordings, rats were challenged s.c. repeatedly once a week with fentanyl (0.075-1.0 mg/kg), sufentanil (0.008 mg/kg), and alfentanil (0.5 mg/kg) as detailed in each experiment. In studies involving anesthetics, rats immunized with either CRM₁ or F₁-CRM₁ were first challenged with dexmedetomidine (0.25 mg/kg, s.c.), whose effects were reversed by atipamezole (1 mg/kg, s.c.). In subsequent weeks, rats were challenged with ketamine (75 mg/kg, s.c.), propofol (100 mg/kg, s.c.), or isoflurane (2%, Drager Vapor 2000, Telford, Pa.). Measurements were obtained at 15, 30, 45, and 60 minutes post-drug administration.

### Example 4C—Vaccination Against Fentanyl does not Interfere with the Pharmacological Activity of Agonists and Antagonists

To test whether anti-fentanyl vaccines would interfere with opioid receptor agonists and antagonists used in treatment of OUD and pain management, rats were immunized with either CRM₁ or F₁-CRM₁ and challenged weekly with a series of agonists or antagonists. Efficacy of opioids was measured by antinociception on the hotplate. In this experiment, rats were challenged sequentially with either oxycodone (2.25 mg/kg, s.c.) or heroin (0.9 mg/kg, s.c.), and antinociception was measured 30 minutes post-challenge. Immediately after, rats were given naloxone (0.1 mg/kg, s.c.) to reverse opioids' effects. Vaccination with F₁-CRM₁ did not interfere with antinociception induced by either oxycodone (FIG. 18A, FIG. 18C) or heroin (FIG. 18B, FIG. 18D), and did not impact the efficacy of naloxone in reversing either oxycodone or heroin effects (FIG. 18A-FIG. 18D). Next, rats were challenged with methadone (2.25 mg/kg, s.c.) and showed that methadone-induced antinociception was not different between CRM₁ and F₁-CRM₁ (FIG. 8E). As a positive control, a final challenge with fentanyl (0.1 mg/kg, s.c.) confirmed that the efficacy of F₁-CRM₁ was preserved against its target opioid (FIG. 18F). These data indicate that vaccination with F₁-CRM₁ does not interfere with the pharmacological activity of oxycodone and methadone, as well as naloxone reversal of the effects of oxycodone and heroin.

In vivo data were further supported by in vitro competitive binding studies demonstrating that polyclonal antibodies induced by conjugates containing the F₁ hapten did not cross-react with methadone, buprenorphine, naltrexone, and naloxone (Table 2C). Hence, it is expected that clinical implementation of vaccines will not impact use of current medications for OUD.

### Example 4D—Vaccination with F₁-Containing Conjugates May Protect from Fatal Overdose from Fentanyl and its Analogs

To determine whether the F₁-CRM₂ conjugate could protect against lethal overdose of fentanyl, a cumulative dosing regimen was administered. Rats were vaccinated i.m. on days 0, 21, 42 and 63 with either F₁-CRM₂ or unconjugated carrier protein. Tail blood was collected on day 69. On day 76, rats were allowed to acclimate to the testing environment for 1 hour, followed by a baseline measurement of oximetry using a MouseOx Plus pulse oximeter (Starr Life Sciences, Oakmont, Pa.). Rats were then given 0.25 mg/kg s.c. fentanyl every 15 minutes to a maximum cumulative dose of 2.25 mg/kg. Prior to each successive dose, rats were measured on the oximeter for respiratory depression and bradycardia. Rats that displayed cardiac or respiratory arrest during the testing period were promptly euthanized via CO₂ and blood and brain were collected for LCMS. If the animal did not go into cardiac or respiratory arrest, a final measurement was taken 15 minutes after the final cumulative dose of 2.25 mg/kg and blood and brain were then collected for LCMS.

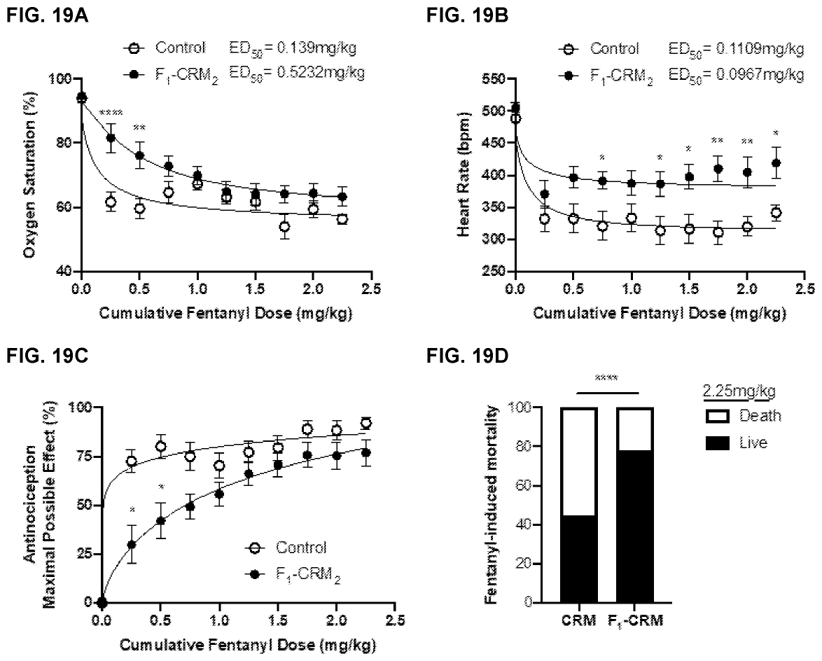

Results are shown in FIG. 19.

These results show the vaccine was effective against opioid-induced respiratory depression, bradycardia, and fatal overdose during cumulative fentanyl dosing.

### Example 4E—Efficacy of Vaccines Containing the F₁ Hapten Against Acetylfentanyl in Rats

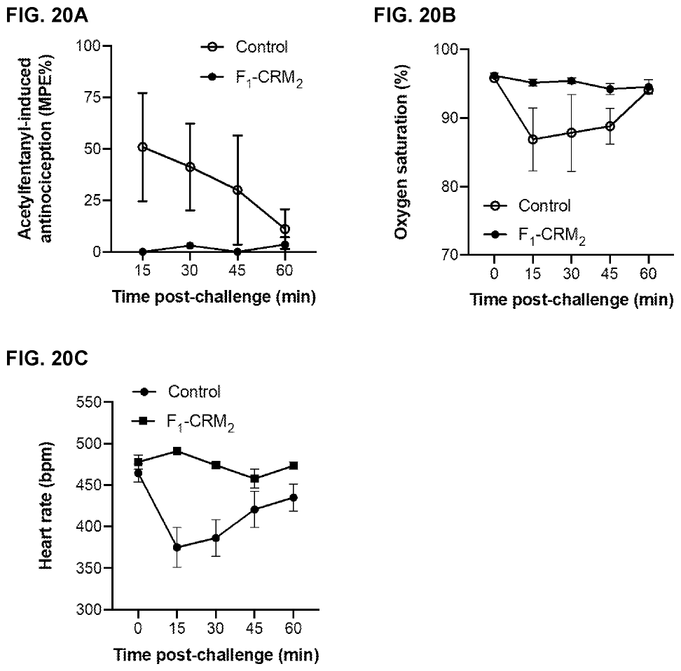

To test the efficacy of vaccines including the F₁ hapten against acetylfentanyl, rats were immunized with control or with F₁-CRM₂ and then were challenged with acetylfentanyl. Results are shown in FIG. 20 ns indicate that vaccination using F₁-CRM₂ may be protective against opioid-induced respiratory depression and bradycardia during cumulative acetylfentanyl dosing.

### Example 5

This Example provides additional information regarding the synthesis of fentanyl hapten F₁, according to Scheme 2 (FIG. 1D).

Reagents and conditions: a) Na(OAc)₃BH, 1,2-DCE, 0° C.-rt, 4 h; b) HCl (4 M in dioxane), DCM, 0° C.-rt, 3 h; e) Methyl glutaryl chloride, Et₃N, DCM, 0° C.-rt, 1 h; LiOH.H₂O, THF/MeOH/H₂O, 6 h.; g) gly₄OMe, BOP, Et₃N, DMF, 0° C.-rt, 8 h; h) LiOH.H₂O, THF/MeOH/H₂O, 30 h.

tert-butyl N-{2-[4-(N-phenylpropanamido)piperidin-1-yl]ethyl}carbamate (2) Norfentanyl 1 (5.46 g, 23.5 mmol) was dissolved in 1,2-DCE (50 mL). N-Boc-2-aminoacetaldehyde (4.5 g, 28.2 mmol) was dissolved in 1,2-DCE (10 mL) and added dropwise to the above solution at 0° C. After that, sodium triacetoxyborohydride (7.43 g, 35.3 mmol) was added to the reaction mixture in three portions at 0° C. The reaction mixture was stirred at rt for 4 h. After completion of the reaction, as indicated by LCMS, the reaction was quenched with saturated aqueous NaHCO₃ (30 mL). The organic layer was separated, and the aqueous layer was extracted with CH₂Cl₂ (3×50 mL). The combined organic layers were washed with saturated NH₄Cl (30 mL) followed by brine (3×50 mL), and dried (Na₂SO₄). The solvents were evaporated under reduced pressure to furnish an oil. The oil was purified on silica using medium pressure chromatography (CHCl₃, MeOH, NH₄OH; 160:18:2) to afford (2, 7.43 g, 84%) as a pale yellow oil. ¹H NMR (300 MHz, CDCl₃) δ 7.43-7.31 (m, 3H), 7.09 (d, J=6.9 Hz, 2H), 4.90 (brs, 1H), 4.67-4.59 (m, 1H), 3.15-3.13 (m, 2H), 2.88-2.85 (m, 2H), 2.40-2.36 (dd, 2H), 2.13-2.06 (m, 2H), 1.95-1.88 (m, 2H), 1.78-1.74 (m, 2H), 1.44-1.26 (m, 11H), 1.03 (t, 3H); ESI MS m/z: calculated for C₂₁H₃₃N₃O₃ 375.25, found 376.23 (M+H)⁺. N-[1-(2-aminoethyl)piperidin-4-yl]-N-phenylpropanamide (3) To an ice cold solution of carbamate 2 (7.43 g, 19.8 mmol) in dry DCM (mL), HCl (15 mL, 4 M solution in dioxane) was added. The reaction was stirred at room temperature for 3 h. The solvent was removed under reduced pressure and excess HCl was removed by flash evaporation with CH₂Cl₂ (3×50 mL) to provide the HCl salt of amine 3 (5.13 g,) as a white solid. The material was used in the next step without further purification.

Methyl 4-({2-[4-(N-phenylpropanamido)piperidin-1-yl]ethyl}carbamoyl)butanoate (4) To a so-lution of amine 3 (0.77 g, 2.8 mmol) in dry CH₂Cl₂ (20 mL), triethyl amine (0.35 mL, 3.5 mmol) and glutaric acid monomethyl ester chloride (0.57 g, 3.5 mmol) was added at 0° C. The reaction was then stirred for 1 h at room temperature. After the completion of the reaction, as indicated by TLC, the reaction was quenched with cold NaHCO₃ solution (20 mL). The layers were separated, and the aqueous layer was extracted with CH₂Cl₂ (3×20 mL). The combined organic layers were washed with brine (3×20 mL), dried (Na₂SO₄), and concentrated under reduced pressure. The residue was subjected to chromatography on silica gel using 0-100% (CHCl₃, CH₃OH, NH₄OH, 80:18:2) in CH₂Cl₂ to furnish amide 4 (0.720 g, 64%) as a white solid. ¹H NMR (300 MHz, CDCl₃) δ 7.51-7.33 (m, 3H), 7.09 (dd, J=7.6, 1.9 Hz, 2H), 6.41-6.26 (m, 1H), 4.76-4.57 (m, 1H), 3.64 (s, 3H), 3.31 (dd, J=11.4, 5.6 Hz, 2H), 2.96 (d, J=11.7 Hz, 2H), 2.48 (t, J=5.9 Hz, 2H), 2.38-2.27 (m, 2H), 2.25-2.07 (m, 4H), 1.97-1.84 (m, 4H), 1.85-1.72 (m, 2H), 1.42 (qd, J=12.4, 3.7 Hz, 2H), 1.01 (t, J=7.4 Hz, 3H) ESI MS m/z: calculated for C₂₂H₃₃N₃O₄ 403.25, found 404.20 (M+H)⁺.

Lithium 4-({2-[4-(N-phenylpropanamido)piperidin-1-yl]ethyl}carbamoyl)butanoate (5)

To a so-lution of the ester 4 (0.72 g, 1.78 mmol) in THF/MeOH/H₂O (50 mL, 1:1:0.5, v/v/v), LiOH.H₂O (0.094 g, 2.23 mmol) was added and the reaction, which resulted, was stirred at room temperature for 6 h. The solvent was evaporated under N₂ flow to provide the lithium salt 5 (0.68 g, quant.) as a white solid. This material was used for the next transformation without any purification. ¹H NMR (300 MHz, CDCl₃) δ 7.49-7.32 (m, 3H), 7.07 (d, J=6.5 Hz, 2H), 4.68-4.46 (m, 1H), 3.66-3.34 (m, 1H), 3.15 (s, 1H), 2.93-2.74 (m, 2H), 2.42-2.24 (m, 2H), 2.09-2.00 (m, 3H), 1.97-1.85 (m, 3H), 1.78-1.61 (m, 4H), 1.29-1.23 (m, 2H), 1.05-0.96 (m, 3H), 0.91-0.82 (m, 3H). ESI MS m/z: calculated for C₂₁H₃₀LiN₃O₄ 395.24, found 396.40 (M+H)⁺.

Methyl-2-[2-(2-{2-[4-({2-[4-(N-phenylpropanamido)piperidin-1-yl]ethyl}carbamoyl) butanamido]acetamido}acetamido)acetamido]acetate (6) The Lithium salt 5 (1.39 g, 3.52 mmol) and BOP (2.33 g, 5.28 mmol) was dissolved in DMF (5 mL) and cooled to 0° C. Triethyl amine (0.73 mL, 0.73 mmol) and gly₄OMe (1.14 g, 4.40 mmol) in DMF (5 mL) was added dropwise to the above reaction at 0° C. The reaction was stirred at room temperature for 8 h. The solvent was removed under reduced pressure and the residue was subjected to chromatography on silica gel using 0-100% CMA80 in DCM to furnish ester 6 (0.88 g, 41%) as a white solid. ¹H NMR (300 MHz, DMSO) δ 8.26 (t, J=5.8 Hz, 1H), 8.16 (dd, J=12.8, 6.0 Hz, 2H), 8.07 (t, J=5.7 Hz, 1H), 7.70 (t, J=5.4 Hz, 1H), 7.50-7.37 (m, 3H), 7.19 (d, J=6.7 Hz, 2H), 4.54-4.34 (m, 1H), 3.84 (d, J=5.9 Hz, 2H), 3.74 (d, J=5.4 Hz, 4H), 3.69 (d, J=5.7 Hz, 2H), 3.62 (s, 3H), 3.08 (dd, J=12.4, 6.4 Hz, 2H), 2.85 (d, J=10.4 Hz, 2H), 2.34-2.22 (m, 2H), 2.10-1.92 (m, 6H), 1.81 (q, J=7.3 Hz, 2H), 1.72-1.60 (m, 4H), 1.19 (dd, J=22.2, 12.7 Hz, 2H), 0.87 (t, J=7.4 Hz, 3H). ESI MS m/z: calculated for C₃₀H₄₅N₇O₈ 631.33, found 632.40 (M+H)⁺.

Lithium2-[2-(2-{2-[4-({2-[4-(N-phenylpropanamido)piperidin-1-yl]ethyl}carbamoyl) butanamido]acetamido}acetamido)acetamido]acetate (F1) To a solution of the ester 6 (1.59 g, 2.45 mmol) in THF/MeOH/H₂O (75 mL, 1:1:0.5, v/v/v), LiOH.H₂O (0.118 g, 2.82 mmol) was added and the reaction, which resulted, was stirred at room temperature. After complete consumption of starting material, as indicated by TLC, the reaction was evaporated to dryness under nitrogen flow to provide the lithium salt F1 (1.48 g, quant.) as a white solid. ¹H NMR (300 MHz, DMSO) δ 8.61-7.94 (m, 3H), 7.56-7.33 (m, 4H), 7.19 (d, J=6.7 Hz, 3H), 4.50-4.33 (m, 1H), 3.72 (d, J=5.7 Hz, 4H), 3.67 (s, 2H), 3.17 (s, 1H), 3.07 (t, J=6.6 Hz, 2H), 2.83 (d, J=11.4 Hz, 2H), 2.25 (t, J=6.6 Hz, 2H), 2.10-1.87 (m, 7H), 1.81 (q, J=7.4 Hz, 2H), 1.72-1.59 (m, 4H), 1.18 (dd, J=22.6, 13.6 Hz, 2H), 0.87 (t, J=7.4 Hz, 3H) ESI MS m/z: calculated for C₂₉H₄₃LiN₇O₈ 633.33, found 634.40 (M+H)⁺.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A fentanyl hapten-carrier conjugate comprising
a fentanyl hapten comprising 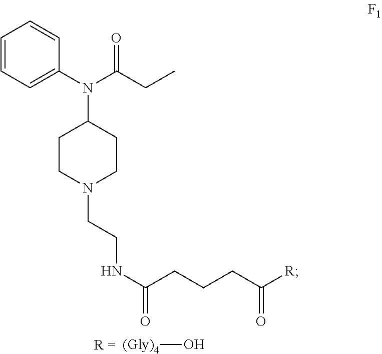
and
an immunogenic carrier, wherein the fentanyl hapten is conjugated to the immunogenic carrier.

2. The fentanyl hapten-carrier conjugate of claim 1,
wherein the F₁ has a differential scanning calorimetry (DSC) thermogram exhibiting an endothermic event having a melt maxima temperature in a range of 110 degrees Celsius (° C.) to 130° C.;
wherein the F₁ has a DSC thermogram exhibiting an endothermic event having a melt maxima temperature in a range of 175° C. to 185° C.;
wherein the F1 has a decompensation temperature of at least 200° C., at least 225° C., or at least 250° C., as measured by thermogravimetric analysis (TGA); or
wherein the F1 has a haptenation ratio to BSA, of at least 10, at least 15, at least 20; or more than 20; or
a combination thereof.

3. The fentanyl hapten-carrier conjugate of claim 1, wherein the immunogenic carrier comprises a carrier selected from bovine serum albumin (BSA), ovalbumin (OVA), keyhole limpet hemocyanin (KLH); CRM; a liposome, tetanus toxoid (TT); a peptide; macro-, micro-, and nano-particles or combinations thereof; a carbon-based particle; a nanocarrier; a protein of viral, bacterial, or synthetic origin; or another immunogenic component; or a mixture or combination thereof.

4. The fentanyl hapten-carrier conjugate of claim 1, wherein the immunogenic carrier comprises KLH.

5. The fentanyl hapten-carrier conjugate of claim 4, wherein the immunogenic carrier comprises GMP grade subunit KLH (sKLH).

6. The fentanyl hapten-carrier conjugate of claim 1, wherein the immunogenic carrier comprises CRM.

7. The fentanyl hapten-carrier conjugate of claim 1, wherein the fentanyl hapten is conjugated to the immunogenic carrier through carbodiimide chemistry.

8. A composition comprising the fentanyl hapten-carrier conjugate of claim 1.

9. The composition of claim 8, wherein the composition further comprises an adjuvant.

10. The composition of claim 9, wherein the adjuvant comprises an aluminum salt-based adjuvant, complete Freund's adjuvant (CFA), incomplete Freund's adjuvant (IFA), a phytol-based adjuvant, a carbohydrate-based adjuvant, a toll like receptor agonist, a oligomerization domain (NOD)-like receptor (NLR) agonist, a RIG-I-like receptor (RLR) agonist, a C-type lectin receptor (CLR) agonist, degradable nanoparticles, or non-degradable nanoparticles, or combinations thereof.

11. A method comprising administering the fentanyl hapten-carrier conjugate of claim 1 to a subject.

12. The method of claim 11, wherein the subject comprises a soldier, a law enforcement professional, a health profession, or a first responder.

13. The method of claim 11, wherein the subject comprises an individual who has been diagnosed with an opioid use disorder or a substance use disorder.

14. The method of claim 11, wherein the subject comprises an individual who has recovered from an opioid use disorder or a substance use disorder.

15. The method of claim 11, wherein the method comprises administering multiple doses of the fentanyl hapten-carrier conjugate to the subject.

16. The method of claim 11, wherein the method comprises administering the fentanyl hapten-carrier conjugate to the subject in combination with an opioid agonist or partial agonist.

17. The method of claim 11, wherein the method comprises administering the fentanyl hapten-carrier conjugate to the subject in combination with an opioid antagonist.

18. The method of claim 11, wherein the method comprises administering the fentanyl hapten-carrier conjugate to the subject in combination with a non-fentanyl opioid or a non-opioid drug hapten-carrier conjugate.

19. The method of claim 11, wherein the subject may be exposed to an opioid or that has been exposed to an opioid or that is suspected of having been exposed to an opioid.

20. The method of claim 19, wherein the opioid comprises fentanyl, sufentanil, acetylfentanyl, and/or carfentanil.

21. A fentanyl hapten comprising 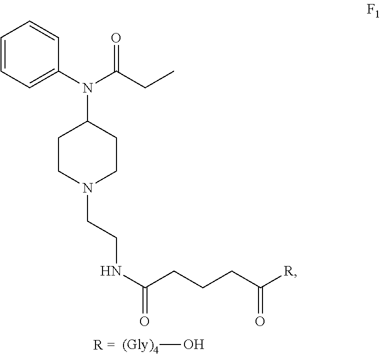
wherein the F₁ has a differential scanning calorimetry (DSC) thermogram exhibiting an endothermic event having a melt maxima temperature in a range of 110 degrees Celsius (° C.) to 130° C.;
wherein the F₁ has a DSC thermogram exhibiting an endothermic event having a melt maxima temperature in a range of 175° C. to 185° C.;
wherein the F1 has a decompensation temperature of at least 250° C., as measured by thermogravimetric analysis (TGA); or
wherein the F1 has a haptenation ratio to BSA, of at least 10, at least 15, at least 20; or more than 20; or
a combination thereof.

22. The fentanyl hapten of claim 21, wherein the F1 has a haptenation ratio to BSA of at least 20.

23. A method of making a fentanyl hapten comprising 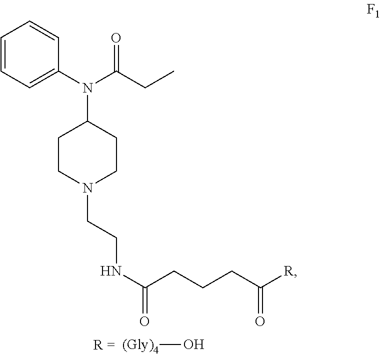
the method comprising the synthesis shown in the following Scheme: [chemical structure]
